(19) United States
(12) Patent Application Publication
(10) Pub. No.: US 2025/0215027 A1
(43) Pub. Date: Jul. 3, 2025

(54) DIPHOSPHINE COMPOUNDS AND COMPLEXES

(71) Applicant: Cancer Research Technology Limited, London (GB)

(72) Inventors: Michelle Therese Ma, London (GB); Ingebjorg Narvestad Hungnes, London (GB); Charlotte Rivas, London (GB); Truc Thuy Pham, London (GB); Paul Gerard Pringle, Bristol (GB); Rachel Elizabeth Nuttall, Bristol (GB)

(73) Assignee: Cancer Research Technology Limited, London (GB)

(21) Appl. No.: 18/682,398

(22) PCT Filed: Aug. 10, 2022
(86) PCT No.: PCT/EP2022/072494

§ 371 Date: Feb. 8, 2024

(30) Foreign Application Priority Data
Aug. 11, 2021 (GB) 2111553.0

(51) Int. Cl. C07F 9/50; A61K 51/04; G01N 33/60

(52) U.S. Cl. CPC C07F 9/5031; A61K 51/0489; C07F 9/5095; G01N 33/60

(57) ABSTRACT

A diphosphine precursor compounds of Formula (I), conjugates thereof of Formula (H) and radionuclide conjugate complexes thereof are disclosed herein. The compounds are advantageous at least because they enable the easy one-step extemporaneous preparation of the corresponding complexes in the clinic in high radiochemical yields and under mild conditions. Also disclosed are the methods of making the compounds and complexes herein along with their uses. The complexes are particularly useful in the field of medicine and diagnosis, such as in medical imaging and targeted payload delivery.

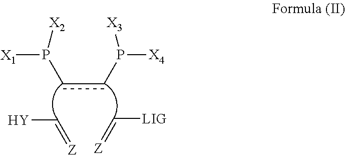

This application claims the benefit of the UK patent application GB 2111553.0 filed 11 Aug. 2021, which is herein incorporated by reference in its entirety.

### FIELD OF THE DISCLOSURE

The present invention relates to compounds and radionuclide complexes, their uses and methods of preparation. The compounds and radionuclide complexes are particularly useful in the imaging, diagnosis and treatment of diseases, such as rheumatoid arthritis and prostate cancer.

### BACKGROUND

In recent years, there has been a shift towards the development of PET radiotracers in preference to SPECT radiotracers. Clinical PET imaging generally provides superior spatial resolution and sensitivity compared to SPECT. However, SPECT radionuclides are generally more widely available, cheaper and longer lived than PET nuclides, and SPECT technology allows for simultaneous imaging using radionuclides with different emission energies.

But advances in detector and collimator technology have led to increased resolution and sensitivity of commercial SPECT scanners, bringing SPECT very close to PET in resolution and sensitivity. γ-Scintigraphy and SPECT cameras are generally more readily accessible than PET facilities (there were 3408 γ-scintigraphy/SPECT cameras and 849 PET scanners in Europe, excluding UK, in 2015/16). The number of clinical γ-scintigraphy and SPECT imaging procedures is also currently higher than that of PET imaging. For example, in England within the NHS from February 2018-February 2019, there were approximately 440,000 γ-scintigraphy/SPECT scans compared to 170,000 PET scans. There is also large international investment in ^{99m}Tc generator production facilities, and new UK cyclotron technology for ^{99m}Tc. These data testify to the continued and future importance of imaging with ^{99m}Tc and other SPECT radionuclides.

However, despite investment and prevalence of SPECT infrastructure, for the last 20 years there has been little parallel development of new, kit-based radiopharmaceutical chemistry for the modern era of molecular imaging, particularly using ^{99m}Tc. The present invention aims to take advantage of the existing prevalent infrastructure and increase access to the benefits of receptor-targeted diagnostic radionuclide imaging via SPECT and gamma-scintigraphy.

Existing “one-pot” ^{99m}Tc radiosynthesis require only generator-produced technetium-99m, commercially available “kit” vials that contain all non-radioactive materials, a syringe, radiation shielding and a Grade A isolator to ensure sterility. The chelators in the kit quantitatively coordinate ^{99m}Tc at low chelator amounts with fast reaction kinetics, enabling routine, sterile and simple radiosynthesis by technicians in clinics. In their current form, these chelator complexes are used for conventional functional imaging (perfusion, renal function, pulmonary ventilation) but crucially are not suitable for conjugation to peptides.

The new chemical platform herein enables a one-step, kit-based radiolabelling of peptides that provides molecular receptor-targeted radiopharmaceuticals. There are existing examples of chelating radionuclides with compounds having targeting ligands in the field of nuclear medicine.

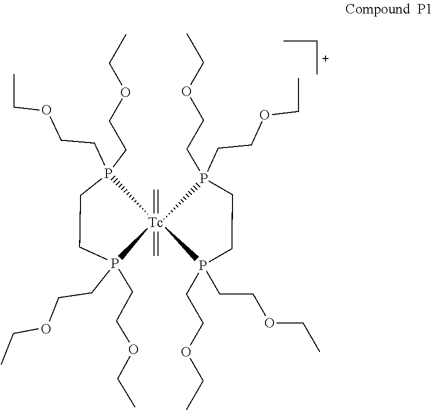

The radiopharmaceutical tetrofosmin is used to image cardiac perfusion. In tetrofosmin (Myoview; Compound P1), two bidentate diphosphines coordinate to a Tc(V) metal centre, with two oxido ligands occupying axial positions. But tetrofosmin uses a very different “diphosphine” chelator, which is not suitable for the receptor-targeted imaging of disease, as it cannot be attached to peptides or proteins.

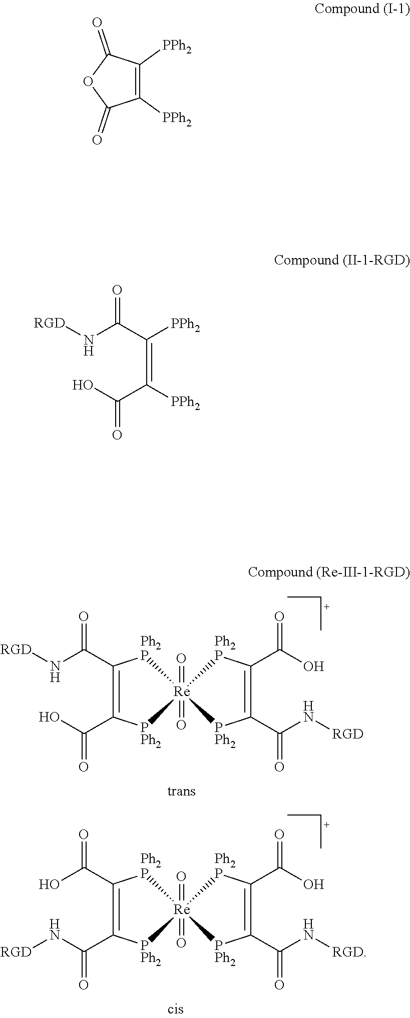

A webpage (https://www.imagingcdt.com/project/bidentate-diphosphine-and-dithiocarbamate-chelators-for-radionuclide-imaging-with-99mtc/; accessed 26 May 2021) describes the intended use of bidentate diphosphine and dithiocarbamate chelators for radionuclide imaging with ^{99m}Tc. The structures of Compounds (I-1) and (II-1-RGD) and Formula P1 are mentioned.

Abstracts/Nuclear Medicine and Biology 72-73/Si (2019) S1-567 PP #66 describes previous work providing bis(diphosphino)maleic anhydride as a bifunctional chelator for ^{99m}Tc.

Neither of the preceding two citations mention substituting the phosphine atoms with substituted aryl groups, heteroaryl groups or cycloalkyl groups or the advantages thereof. Only the metals (M) Re and Tc and the peptide RGD are mentioned. There is no mention of the compounds or advantages of the present invention.

J. Chem. Soc., Dalton Trans., 1997, 855-862 describes chelating diphosphine 2,3-bis(diphenylphosphino)maleic anhydride Compound (I-1) reacted with CuCl to give a tetrahedral structure.

Chem. Commun. 1996, No. 10, 1093 describes copper(I) bis(diphosphine) complexes as a basis for radiopharmaceuticals for positron emission tomography and targeted radiotherapy.

US20110033379A1 describes radio-labelled materials and methods of making and using the same. However, it relies on nitrogen atoms, and sometimes sulfur atoms, in the metal chelating moiety to chelate a radionuclide. Diphosphine groups are not mentioned.

WO2003086476A1 describes technetium-labelled rotenone derivatives and methods of use thereof, particularly in cardiac imaging. However, it relies on nitrogen atoms in the metal chelating moiety to form a complex that includes a radionuclide and a rotenone derivative. Diphosphine groups are not mentioned.

WO2010108125A2 describes prostate specific membrane antigen (PSMA) binding compounds. However, it relies on nitrogen atoms in the metal chelating moiety to chelate a radionuclide. Diphosphine groups are not mentioned.

The inventors have identified a new chemical platform that enables one-step, kit-based radiolabelling of targeting ligands.

### SUMMARY OF THE DISCLOSURE

In the broadest sense, the present invention provides a chemical platform to enable one-step, kit-based radiolabelling of targeting ligands. The radiolabelled complexes may then be used in medicine, such as for imaging or disease treatment. A diphosphine compound is used to unite a radioactive isotope with a biological ligand to simultaneously exploit their advantageous properties.

### Conjugated Diphosphine Precursor Compound

In a first aspect of the invention, there is provided a conjugated diphosphine precursor compound according to Formula (II) that is suitable for preparing a conjugated radiolabelled agent (e.g. a conjugated radiolabelled diphosphine complex);

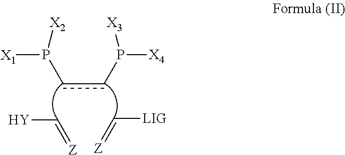

wherein;
each Z is independently O or S;
Y is NH or O;
X₁, X₂, X₃ and X₄ are each independently a substituted or unsubstituted C₃-C₈ aryl group, a substituted or unsubstituted 5- to 8-membered heteroaryl group or a substituted or unsubstituted C₃-C₈ cycloalkyl group wherein each substituent is selected from the group consisting of a C₁-C₄alkyl group, C₃-C₁₂aryl or heteroaryl group, a C₁-C₄ acylamido group, a sulfylhydro group, a C₁-C₄ alkylthio group, a C₁-C₄(di)alkylphosphino group, a hydroxy group, a C₁-C₄alkoxy group, a carboxyl group, a C₁-C₄(di)alkylamino group and a C₁-C₄alkoxy-(CH₂CH₂O)ₙ group wherein n is 1, 2, 3, 4, 5, 6, 7, 8, 9 or 10;
the shortest linear chain of carbon atoms between the two Z groups is 4 to 7;
LIG comprises a ligand with a binding motif corresponding to a biological target; and the compound is not

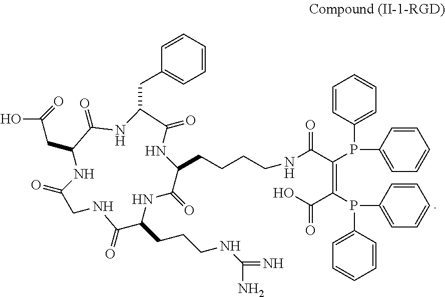

Each variable group LIG, Z, Y, X₁, X₂, X₃ and X₄ in Formula (II), and any subgroups thereof, may also independently be selected from and combined with any of the definitions provided anywhere herein. The disclaimer of Compound (II-1-RGD) also applies to the subformulae of Formula (II) herein. It may be formed from the diphosphine precursor compound of the first aspect above.

LIG comprises a binding motif (i.e. a targeting ligand) that is selective for biological targets, such as enzymes or receptors, due to forming interactions specific to that target. In some cases, LIG comprises a peptide or carbohydrate ligand with a binding motif corresponding to a biological target. In some instances, LIG comprises a prostate specific membrane antigen targeting ligand (PSMAt), cyclic(Arg-Gly-Asp-dPhe-Lys) (RGD), pentixafor peptide, a minigastrin peptide analogue for targeting cholecystokinin-2 receptor, a c-Met-targeting peptide, an alpha-MSH peptide, a bisphosphonate, a folate or a carbohydrate. In some cases, LIG comprises a prostate specific membrane antigen targeting ligand (PSMAt) or cyclic(Arg-Gly-Asp-dPhe-Lys) (RGD). PSMAt targets prostate specific membrane antigen. The PSMAt may be provided, for example, as part of the group PSMAt1 described herein. RGD targets the αvβ3-integrin receptor, (which is over-expressed in neovasculature, inflammation processes and cancer cells). Pentixafor peptide targets CXCR-4. Minigastrin peptide analogues target cholecystokinin-2 receptor. Alpha-MSH targets MCR1 in melanoma. Bisphosphonates target mineralisation processes in bone metastases. Folate targets folate receptor. LIG is preferably PSMAt1. LIG preferably has a molecular weight of 50 g/mol or more, such as 100 g/mol or more or 200 g/mol or more. LIG preferably has a molecular weight of 3,000 g/mol or less, such as 2,000 g/mol or less or 1,000 g/mol or less. LIG is not H, OH, NH₂ or NHBn. LIG preferably comprises 10 or more atoms, such as 15 or more atoms or 20 or more atoms. LIG preferably comprises 100 or fewer atoms, such as 75 or fewer atoms or 50 or fewer atoms.

In some cases, LIG is attached via a nitrogen atom that forms an amide bond with group Z so that Z is O. In other cases, LIG is attached via a nitrogen atom that forms a (thio)amide bond with the corresponding group Z so that Z is S. LIG may comprise a PEG linker moiety. LIG may comprise a terminal moiety having a urea group and three carboxylic acid groups. The carboxylic acid groups may be derived from amino acids. The terminal moiety may be two glutamic acid groups linked by a middle urea group; or a lysine group and a glutamic acid group linked by a urea group. The terminal moiety of LIG may be PSMAt.

In some cases, there is provided a conjugated diphosphine precursor compound according to Formula (IIa) that is suitable for preparing a conjugated radiolabelled agent:

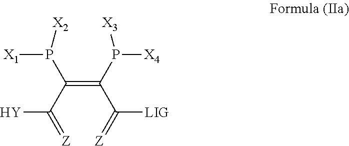

wherein;
each Z is independently O or S;
Y is NH or O;
X₁, X₂, X₃ and X₄ are each independently a substituted or unsubstituted C₃-C₈ aryl group wherein each substituent is selected from the group consisting of a C₁-C₄alkyl group, a C₁-C₄alkoxy group, a C₁-C₄(di)alkylamino group and a C₁-C₄alkoxy-(CH₂CH₂O)ₙ group wherein n is 1, 2, 3, 4, 5, 6, 7, 8, 9 or 10; and
LIG comprises a peptide or carbohydrate ligand with a binding motif corresponding to a biological target.

Each variable group LIG, Z, Y, X₁, X₂, X₃ and X₄ in Formula (IIa), and any subgroups thereof, may also independently be selected from and combined with any of the definitions provided anywhere herein.

In some instances, there is provided a conjugated diphosphine precursor compound according to Formula (IIb) and/or Formula (IIc) that is suitable for preparing a conjugated radiolabelled agent:

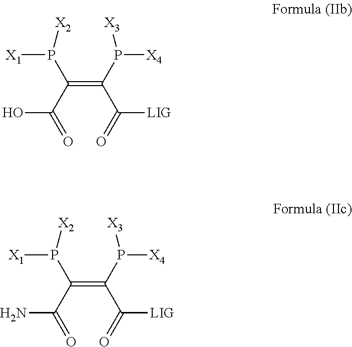

wherein;
X₁, X₂, X₃ and X₄ are each independently a substituted or unsubstituted phenyl group wherein each substituent is selected from the group consisting of a C₁-C₄alkyl group, a C₁-C₄alkoxy group, a C₁-C₄(di)alkylamino group and a C₁-C₄alkoxy-(CH₂CH₂O)n group wherein n is 1, 2, 3, 4, 5, 6, 7, 8, 9 or 10; and
LIG comprises a prostate specific membrane antigen targeting ligand (PSMAt), cyclic(Arg-Gly-Asp-dPhe-Lys) (RGD), pentixafor peptide, a minigastrin peptide analogue for targeting cholecystokinin-2 receptor, a c-Met-targeting peptide, an alpha-MSH peptide, a bisphosphonate, a folate or a carbohydrate.

RGD herein is according to the following formula, where the wavy line signifies the attachment bond;

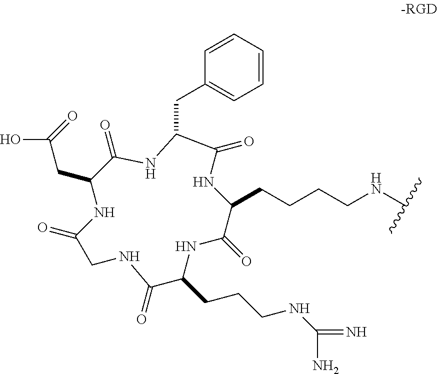

PSMAt herein may be attached via an amide bond to a PEG linker moiety that in turn is attached via an amide with group Z. In some cases, the PEG linker comprises 1, 2, 3, 4, 5, 6, 7, 8, 9 or 10 repeat units. The PSMAt may be provided as a terminal group in PSMAt1, which is according to the following formula, wherein the wavy line signifies the attachment point of LIG;

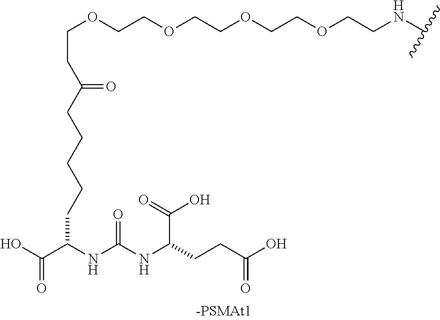

Each variable group LIG, X₁, X₂, X₃ and X₄ in Formula (IIb) or Formula (IIc), and any subgroups thereof, may also independently be selected from and combined with any of the definitions provided anywhere herein.

In some instances, there is provided a conjugated diphosphine precursor compound according to Formula (IIb) and/or Formula (IIc) that is suitable for preparing a conjugated radiolabelled agent wherein; X₁, X₂, X₃ and X₄ are each independently a phenyl group having one, two or three substituents selected from the group consisting of methyl, ethyl, n-propyl, isopropyl, cyclopropyl, n-butyl, isobutyl, t-butyl, cyclobutyl, methoxy, ethoxy, ethenyl, dimethylamino and MeO(CH₂CH₂O)n, wherein n is 1, 2, 3, 4, 5, 6, 7, 8, 9 or 10; and LIG comprises a prostate specific membrane antigen targeting ligand (PSMAt) or cyclic(Arg-Gly-Asp-dPhe-Lys) (RGD).

In some cases, there is provided a diphosphine precursor compound according to Formula (IIb) that is suitable for preparing a conjugated radiolabelled agent wherein; X₁, X₂, X₃ and X₄ are each independently a phenyl group substituted only in the para position by a substituent selected from the group consisting of methyl, ethyl, n-propyl, isopropyl, cyclopropyl, n-butyl, isobutyl, t-butyl, cyclobutyl, methoxy, ethoxy, ethenyl, dimethylamino and MeO(CH₂CH₂O)ₘ, wherein n is 1, 2, 3, 4, 5, 6, 7, 8, 9 or 10; and LIG comprises a prostate specific membrane antigen targeting ligand (PSMAt) or cyclic(Arg-Gly-Asp-dPhe-Lys) (RGD).

In some cases, the conjugated diphosphine precursor compound is according to Formula (IIb), wherein X₁, X₂, X₃ and X₄ and LIG are according to a line in the following table;

TABLE 1

| Compound | X₁, X₂, X₃ and X₄ | LIG |
|---|---|---|
| Compound (II-2-RGD) | p-tolyl | RGD |
| Compound (II-3-RGD) | m-tolyl | RGD |
| Compound (II-4-RGD) | o-tolyl | RGD |
| Compound (II-5-RGD) | 2,3-xylyl | RGD |
| Compound (II-6-RGD) | 2,4-xylyl | RGD |
| Compound (II-7-RGD) | 2,5-xylyl | RGD |
| Compound (II-8-RGD) | 2,6-xylyl | RGD |
| Compound (II-9-RGD) | 3,4-xylyl | RGD |
| Compound (II-10-RGD) | 3,5-xylyl | RGD |
| Compound (II-11-RGD) | p-methoxyphenyl | RGD |
| Compound (II-12a-RGD) | 4-(MeO(CH₂CH₂O))phenyl | RGD |
| Compound (II-12b-RGD) | 4-(MeO(CH₂CH₂O)₂)phenyl | RGD |
| Compound (II-12c-RGD) | 4-(MeO(CH₂CH₂O)₃)phenyl | RGD |
| Compound (II-13-RGD) | 4-dimethylaminophenyl | RGD |
| Compound (II-14-RGD) | o-methoxyphenyl | RGD |
| Compound (II-1-PSMAt1) | phenyl | PSMAt1 |
| Compound (II-2-PSMAt1) | p-tolyl | PSMAt1 |
| Compound (II-3-PSMAt1) | m-tolyl | PSMAt1 |
| Compound (II-4-PSMAt1) | o-tolyl | PSMAt1 |
| Compound (II-5-PSMAt1) | 2,3-xylyl | PSMAt1 |
| Compound (II-6-PSMAt1) | 2,4-xylyl | PSMAt1 |
| Compound (II-7-PSMAt1) | 2,5-xylyl | PSMAt1 |
| Compound (II-8-PSMAt1) | 2,6-xylyl | PSMAt1 |
| Compound (II-9-PSMAt1) | 3,4-xylyl | PSMAt1 |
| Compound (II-10-PSMAlt) | 3,5-xylyl | PSMAt1 |
| Compound (II-11-PSMAt1) | p-methoxyphenyl | PSMAt1 |
| Compound (II-12a-PSMAt1) | 4-(MeO(CH₂CH₂O))phenyl | PSMAt1 |
| Compound (II-12b-PSMAt1) | 4-(MeO(CH₂CH₂O)₂)phenyl | PSMAt1 |
| Compound (II-12c-PSMAt1) | 4-(MeO(CH₂CH₂O)₃)phenyl | PSMAt1 |
| Compound (II-13-PSMAt1) | 4-dimethylaminophenyl | PSMAt1 |
| Compound (II-14-PSMAt1) | o-methoxyphenyl | PSMAt1 |

### Diphosphine Precursor Compound

In a second aspect there is provided a diphosphine precursor compound according to Formula (I) that is suitable for preparing a conjugated radiolabelled agent (e.g. a conjugated radiolabelled diphosphine complex):

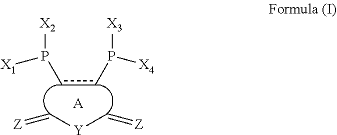

wherein
ring A is a 5, 6, 7 or 8 membered ring;
each Z is independently O or S;
Y is NH or O;
X₁, X₂, X₃ and X₄ are each independently a substituted or unsubstituted C₃-C₈ aryl group, a substituted or unsubstituted 5 to 8-membered heteroaryl group or a substituted or unsubstituted C₃-C₈ cycloalkyl group wherein any substituents selected from the group consisting of a C₁-C₄alkyl group, C₅-C₁₂aryl or heteroaryl group, a C₁-C₄ acylamido group, a sulfylhydro group, a C₁-C₄ alkylthio group, a C₁-C₄(di)alkylphosphino group, a hydroxy group, a C₁-C₄alkoxy group, a carboxyl group, a C₁-C₄(di)alkylamino group and a C₁-C₄alkoxy-(CH₂CH₂O) group wherein n is 1, 2, 3, 4, 5, 6, 7, 8, 9 or 10 and
the compound is not

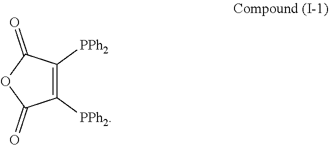

Each variable group A, Z, Y, X₁, X₂, X₃ and X₄ in Formula (I), and any subgroups thereof, may also independently be selected from and combined with any of the definitions provided anywhere herein. The disclaimer of Compound (I-1) also applies to the subformulae of Formula (I) herein. The single bond with a dashed line in Formula (I) indicates that the bond may be a single C—C bond or a double C═C bond.

One advantage of the present invention is that the A ring enables conjugation with a ligand moiety of choice via a ring opening reaction to prime the compound for binding to a radionuclide in the clinic (i.e. at a hospital, radiopharmacy or production unit) immediately prior to use. The diphosphine motif subsequently enables an easy and efficient extemporaneous one-step complexation of the chosen radioactive isotope in physiologically compatible solutions in the clinic shortly before use.

Another advantage is that having a substituted aryl group or a substituted or unsubstituted heteroaryl group provides improved efficiency and radiochemical yields of the corresponding conjugated diphosphine precursor compounds compared to similar known compounds. The radiolabelling can also be conducted under milder conditions and used without further purification.

Another advantage of the present invention is that specific substitution pattern of the phosphine ligands allows for precise electronic tuning to improve the efficiency and radiochemical yield of the corresponding conjugated diphosphine precursor compounds in view of the specific radionuclide or kit that is being used. It has been found in particular that electron donating substituent options for the X₁, X₂, X₃ and X₄ groups provide this advantage. Furthermore, the substitution pattern of the phosphine ligands also allow for tuning of the hydrophobicity or hydrophilicity of the final complexes, modifying their in vivo properties, such as their biodistribution or pharmacokinetics.

Another advantage of the present invention is that the stoichiometry of the complexes formed by the diphosphine moieties provides two copies of the targeting ligand per complex. This provides a higher tumour uptake compared to their monomeric homologues due to their higher affinity for the target receptors. It also means that the complex has a higher affinity for receptor targets than the non-complexed single targeting ligand. Without being bound by any theory, it is believed that any excess targeting ligand therefore does not compromise the binding of the tracer complex in vivo thereby removing the need to perform an additional purification step.

In some cases, A is a 5 or 6-membered ring. A may be aryl group. A may be a 5-membered ring. A may be an unsaturated non-aromatic ring. A may be maleic anhydride.

In some cases, Y is NH or O and each Z is O.

In some cases, X₁, X₂, X₃ and X₄ are each substituted aryl groups. In other cases, X₁, X₂, X₃ and X₄ are each substituted or unsubstituted heteroaryl groups. In some cases, X₁, X₂, X₃ and X₄ are each substituted phenyl groups, optionally substituted only in the para position. In some cases, X₁, X₂, X₃ and X₄ are each substituted or unsubstituted cyclohexyl groups, optionally substituted only in the para position. In some cases, X₁, X₂, X₃ and X₄ donate more electron density to the phosphine than a phenyl group. In some instances, each of X₁, X₂, X₃ and X₄ is substituted with one or more C₁-C₄alkyl groups, optionally wherein each alkyl group is selected from the list consisting of methyl, ethyl, propyl, isopropyl, cyclopropyl, n-butyl, isobutyl, tert-butyl and cyclobutyl. In some instances, X₁, X₂, X₃ and X₄ are each independently substituted with one to three substituents, one or two substituents, or only one substituent. In some cases, X₁, X₂, X₃ and X₄ are each substituted in the same position(s). In some instances, X₁, X₂, X₃ and X₄ each have the same substituent group(s). In some cases, X₁, X₂, X₃ and X₄ have the same substituent group(s) in the same position(s). In some instances, X₁, X₂, X₃ and X₄ are the same.

In some cases, there is provided a diphosphine precursor compound according to Formula (Ia) that is suitable for preparing a conjugated radiolabelled agent:

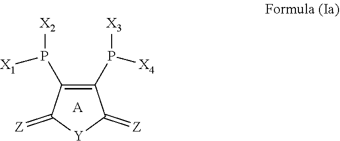

wherein
each Z is independently O or S;
Y is NH or O;
X₁, X₂, X₃ and X₄ are each independently a substituted C₃-C₈ aryl group having one or more substituents selected from the group consisting of a C₁-C₄alkyl group, a C₁-C₄alkoxy group, a C₁-C₄(di)alkylamino group and a C₁-C₄alkoxy-(CH₂CH₂O) group wherein n is 1, 2, 3, 4, 5, 6, 7, 8, 9 or 10.

Each variable group Z, Y, X₁, X₂, X₃ and X₄ in Formula (Ia), and any subgroups thereof, may also independently be selected from and combined with any of the definitions provided anywhere herein.

In some cases, there is provided a diphosphine precursor compound according to Formula (Ib) and/or (Ic) that is suitable for preparing a conjugated radiolabelled agent:

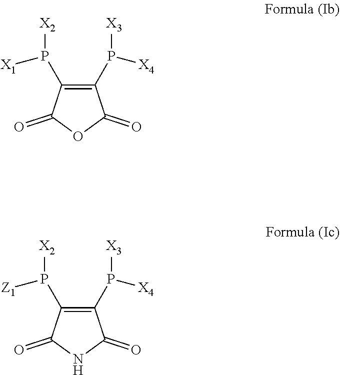

wherein;
X₁, X₂, X₃ and X₄ are each independently a phenyl group having one or more substituents selected from the group consisting of a C₁-C₄alkyl group, a C₁-C₄alkoxy group, a C₁-C₄(di)alkylamino group and a C₁-C₄alkoxy-(CH₂CH₂O)ₙ group wherein n is 1, 2, 3, 4, 5, 6, 7, 8, 9 or 10.

Each variable group X₁, X₂, X₃ and X₄ in Formula (Ib) or Formula (Ic), and any subgroups thereof, may also independently be selected from and combined with any of the definitions provided anywhere herein.

In some instances, there is provided a diphosphine precursor compound according to Formula (Ib) and/or Formula (Ic) that is suitable for preparing a conjugated radiolabelled agent wherein; X₁, X₂, X₃ and X₄ are each independently a phenyl group having one, two or three substituents selected from the group consisting of methyl, ethyl, n-propyl, isopropyl, cyclopropyl, n-butyl, isobutyl, t-butyl, cyclobutyl, methoxy, ethoxy, ethenyl, dimethylamino and MeO(CH₂CH₂O)ₙ, wherein n is 1, 2, 3, 4, 5, 6, 7, 8, 9 or 10.

In some cases, there is provided a diphosphine precursor compound according to Formula (Ib) that is suitable for preparing a conjugated radiolabelled agent wherein X₁, X₂, X₃ and X₄ are each independently a phenyl group substituted only in the para position by a substituent selected from the group consisting of methyl, ethyl, n-propyl, isopropyl, cyclopropyl, n-butyl, isobutyl, t-butyl, cyclobutyl, methoxy, ethoxy, ethenyl, dimethylamino and MeO(CH₂CH₂O)ₙ, wherein n is 1, 2, 3, 4, 5, 6, 7, 8, 9 or 10.

In some cases, the diphosphine precursor compound is according to Formula (Ib), wherein X₁, X₂, X₃ and X₄ are according to a line in the following table;

TABLE 2

| Compound | X₁, X₂, X₃ and X₄ |
|---|---|
| Compound (I-2) | p-tolyl |
| Compound (I-3) | m-tolyl |
| Compound (I-4) | o-tolyl |
| Compound (I-5) | 2,3-xylyl |
| Compound (I-6) | 2,4-xylyl |
| Compound (I-7) | 2,5-xylyl |
| Compound (I-8) | 2,6-xylyl |
| Compound (I-9) | 3,4-xylyl |
| Compound (I-10) | 3,5-xylyl |
| Compound (I-11) | p-methoxyphenyl |
| Compound (I-12a) | 4-(MeO(CH₂CH₂O))phenyl |
| Compound (I-12b) | 4-(MeO(CH₂CH₂O)₂)phenyl |
| Compound (I-12c) | 4-(MeO(CH₂CH₂O)₃)phenyl |
| Compound (I-13) | 4-dimethylaminophenyl |
| Compound (I-14) | o-methoxyphenyl |

wherein o means ortho, m means meta and p means para.

In some cases, the diphosphine precursor compound is compound (I-2).

### Radiolabelled Conjugated Diphosphine Complex

In a third aspect there is provided a radiolabelled diphosphine complex that may be formed from the conjugated diphosphine precursor compound of the first aspect above.

The complex comprises at least two conjugated diphosphine precursor compounds of the second aspect of the invention as ligands that are co-ordinated with one or more radionuclides selected from ^{99m}Tc, ²¹²Pb ²¹²Bi, ²¹³Bi, ¹⁸⁶Re, ¹⁸⁸Re, ⁸⁹Zr, ⁶⁷Ga, ⁶⁸Ga, ⁶⁷Cu, ⁶⁴Cu, ⁶²Cu, ⁶¹Cu, ⁶⁰Cu, ⁶²Zn and ⁵²Mn; and
the complex is not;

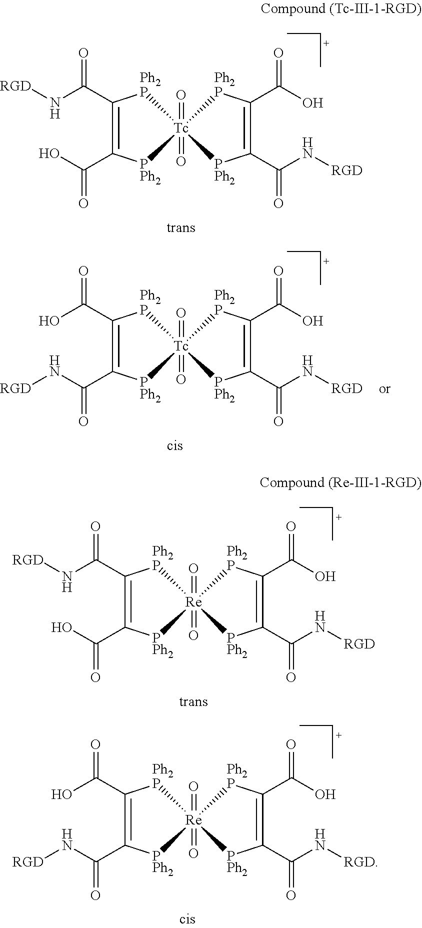

Preferably the one or more radionuclides are selected from ^{99m}Tc, ⁸⁶Re and ⁸⁸Re. The radionuclide may also be selected from selected from ⁶⁷Cu, ⁶⁴Cu, ⁶²Cu, ⁶¹Cu and ⁶¹Cu. The at least two conjugated diphosphine precursor compounds may be the same. Optionally, the complex has only two conjugated diphosphine precursor compounds as ligands. Optionally, the conjugated diphosphine precursor compounds act as bidentate ligands and co-ordinate the radionuclide via the two phosphine atoms.

In some cases, the complex exists as either;
(a) Formula (M-III-trans) or Formula (M-III-cis) or a mixture thereof; [symbol]
   Formula (M-III-trans) Formula (M-IT-cis)
   wherein M is a radionuclide selected from one or more of ^{99m}Tc, ¹⁸⁶Re and ¹⁸⁸Re; or
(b) Formula (C-III-A) or Formula (C-III-B) or a mixture thereof;

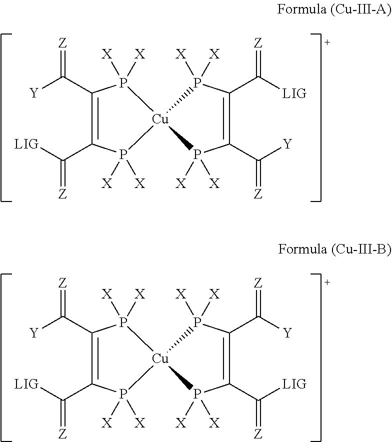

wherein Cu is selected from ⁶⁷Cu, 64Cu, ⁶²Cu, ⁶¹Cu and ⁶⁰Cu;
   and in either case each of X, Y, Z and LIG are as defined in any of the second aspects of the invention. X is the same for each instance and represents X¹, X², X³ and X⁴ of the second aspect when they are all the same value.

Unless otherwise indicated, reference to Formula (M-III-cis/trans) herein, and specific compounds thereof, includes all isomers.

Each variable group LIG, Z, Y and X in Formula (M-III-trans) and Formula (M-III-cis), and any subgroups thereof, may also independently be selected from and combined with any of the definitions provided anywhere herein. In particular, X may also be selected from any of the definitions of X₁, X₂, X₃ and X₄ provided herein. The disclaimers of Compound (Tc-III-1-RGD) and Compound (Re-III-1-RGD) also applies to the subformulae of Formula (M-III-cis/trans) herein.

The present invention may employ the radionuclides alone or in combinations. For example, one commonly used combination is ^{186/188}Re. In general, technetium isotopes are employed for imaging purposes, rhenium isotopes for therapeutic purposes and copper isotopes for both imaging and therapy.

The isomers of the complex may exist separately or as a mixture. The mixture of, for example, the complexes formed using ^{99m}Tc, ¹⁸⁶Re or ¹⁸⁸Re, is typically about 1:1 cis/trans, but other mixture ratios are envisaged.

In some cases, the radiolabelled conjugated diphosphine complex is either;
(a) according to Formula (M-IIIa-trans) or Formula (M-IIIa-cis) or a mixture thereof

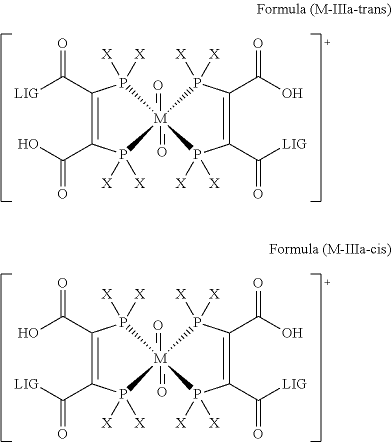

wherein M is a radionuclide selected from one or more of ^{99m}Tc, ¹⁸⁶Re and ⁸⁸Re; or
(b) according to Formula (M-IIIb-trans) or Formula (M-IIIb-cis) or a mixture thereof

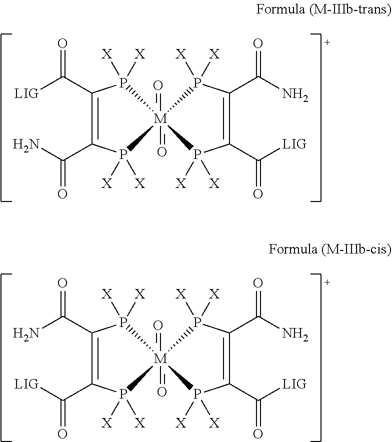

wherein M is a radionuclide selected from one or more of ^{99m}Tc, ¹⁸⁶Re and ⁸⁸Re; or
(c) according to Formula (Cu-IIIc-A) or Formula (Cu-IIIc-B) or a mixture thereof;

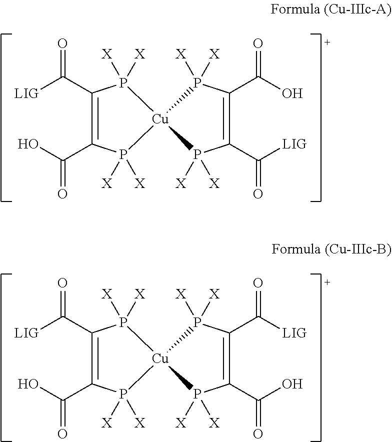

wherein Cu is selected from ⁶⁷Cu, ⁶⁴Cu, ⁶²Cu, ⁶¹Cu and ⁶⁰Cu; or
(d) according to Formula (Cu-IIId-A) or Formula (Cu-IIId-B) or a mixture thereof;

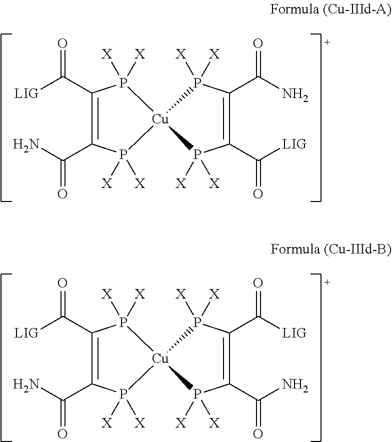

wherein Cu is selected from ⁶⁷Cu, 64Cu, ⁶²Cu, ⁶¹Cu and ⁶⁰Cu;
and wherein;
   X is a phenyl group having one or more substituents selected from the group consisting of a C₁-C₄alkyl group and a C₁-C₄alkoxy group; and
   LIG comprises a prostate specific membrane antigen targeting ligand (PSMAt), cyclic(Arg-Gly-Asp-dPhe-Lys) (RGD), pentixafor peptide, a minigastrin peptide analogue for targeting cholecystokinin-2 receptor, a c-Met-targeting peptide, an alpha-MSH peptide, a bisphosphonate, a folate or a carbohydrate.

Each variable group LIG, Z, Y and X in Formula (M-IIIa-trans) and Formula (M-IIIa-cis), Formula (M-IIIb-trans) and Formula (M-IIIb-cis), Formula (M-IIIc-A) or Formula (M-IIIc-B) or Formula (M-IIId-A) or Formula (M-IIId-B) and any sub groups thereof, may also independently be selected from and combined with any of the definitions provided anywhere herein. In particular, X may also be selected from any of the definitions of X₁, X₂, X₃ and X₄ provided herein.

In some cases, there is provided a complex according to (a) Formula (M-IIIa-trans) or Formula (M-IIIa-cis) or a mixture thereof; or (b) Formula (M-IIIb-trans) or Formula (M-IIIb-cis) or a mixture thereof, wherein M, X and LIG are according to a line in the following table;

TABLE 3

| Compound | M | X | LIG |
|---|---|---|---|
| Compound (Tc-III-2-RGD) | ^{99m}Tc | p-tolyl | RGD |
| Compound (Tc-III-3-RGD) | ^{99m}Tc | m-tolyl | RGD |
| Compound (Tc-III-4-RGD) | ^{99m}Tc | o-tolyl | RGD |
| Compound (Tc-III-5-RGD) | ^{99m}Tc | 2,3-xylyl | RGD |
| Compound (Tc-III-6-RGD) | ^{99m}Tc | 2,4-xylyl | RGD |
| Compound (Tc-III-7-RGD) | ^{99m}Tc | 2,5-xylyl | RGD |
| Compound (Tc-III-8-RGD) | ^{99m}Tc | 2,6-xylyl | RGD |
| Compound (Tc-III-9-RGD) | ^{99m}Tc | 3,4-xylyl | RGD |
| Compound (Tc-III-10-RGD) | ^{99m}Tc | 3,5-xylyl | RGD |
| Compound (Tc-III-11-RGD) | ^{99m}Tc | p-methoxyphenyl | RGD |
| Compound (Tc-III-12a-RGD) | ^{99m}Tc | 4-(MeO(CH₂CH₂O))phenyl | RGD |
| Compound (Tc-III-12b-RGD) | ^{99m}Tc | 4-(MeO(CH₂CH₂O)₂)phenyl | RGD |
| Compound (Tc-III-12c-RGD) | ^{99m}Tc | 4-(MeO(CH₂CH₂O)₃)phenyl | RGD |
| Compound (Tc-III-13-RGD) | ^{99m}Tc | 4-dimethylaminophenyl | RGD |
| Compound (Tc-III-14-RGD) | ^{99m}Tc | o-methoxyphenyl | RGD |
| Compound (Tc-III-1-PSMAt1) | ^{99m}Tc | phenyl | PSMAt1 |
| Compound (Tc-III-2-PSMAt1) | ^{99m}Tc | p-tolyl | PSMAt1 |
| Compound (Tc-III-3-PSMAt1) | ^{99m}Tc | m-tolyl | PSMAt1 |
| Compound (Tc-III-4-PSMAt1) | ^{99m}Tc | o-tolyl | PSMAt1 |
| Compound (Tc-III-5-PSMAt1) | ^{99m}Tc | 2,3-xylyl | PSMAt1 |
| Compound (Tc-III-6-PSMAt1) | ^{99m}Tc | 2,4-xylyl | PSMAt1 |
| Compound (Tc-III-7-PSMAt1) | ^{99m}Tc | 2,5-xylyl | PSMAt1 |
| Compound (Tc-III-8-PSMAt1) | ^{99m}Tc | 2,6-xylyl | PSMAt1 |
| Compound (Tc-III-9-PSMAt1) | ^{99m}Tc | 3,4-xylyl | PSMAt1 |
| Compound (Tc-III-10-PSMAt1) | ^{99m}Tc | 3,5-xylyl | PSMAt1 |
| Compound (Tc-III-11-PSMAt1) | ^{99m}Tc | p-methoxyphenyl | PSMAt1 |
| Compound (Tc-III-12a-PSMAt1) | ^{99m}Tc | 4-(MeO(CH₂CH₂O))phenyl | PSMAt1 |
| Compound (Tc-III-12b-PSMAt1) | ^{99m}Tc | 4-(MeO(CH₂CH₂O)₂)phenyl | PSMAt1 |
| Compound (Tc-III-12c-PSMAt1) | ^{99m}Tc | 4-(MeO(CH₂CH₂O)₃)phenyl | PSMAt1 |
| Compound (Tc-III-13-PSMAt1) | ^{99m}Tc | 4-dimethylaminophenyl | PSMAt1 |
| Compound (Tc-III-14-PSMAt1) | ^{99m}Tc | o-methoxyphenyl | PSMAt1 |
| Compound (Re-III-2-RGD) | ¹⁸⁸Re | p-tolyl | RGD |
| Compound (Re-III-3-RGD) | ¹⁸⁸Re | m-tolyl | RGD |
| Compound (Re-III-4-RGD) | ¹⁸⁸Re | o-tolyl | RGD |
| Compound (Re-III-5-RGD) | ¹⁸⁸Re | 2,3-xylyl | RGD |
| Compound (Re-III-6-RGD) | ¹⁸⁸Re | 2,4-xylyl | RGD |
| Compound (Re-III-7-RGD) | ¹⁸⁸Re | 2,5-xylyl | RGD |
| Compound (Re-III-8-RGD) | ¹⁸⁸Re | 2,6-xylyl | RGD |
| Compound (Re-III-9-RGD) | ¹⁸⁸Re | 3,4-xylyl | RGD |
| Compound (Re-III-10-RGD) | ¹⁸⁸Re | 3,5-xylyl | RGD |
| Compound (Re-III-11-RGD) | ⁸Re | p-methoxyphenyl | RGD |
| Compound (Re-III-12a-RGD) | ¹⁸⁸Re | 4-(MeO(CH₂CH₂O))phenyl | RGD |
| Compound (Re-III-12b-RGD) | ¹⁸⁸Re | 4-(MeO(CH₂CH₂O)₂)phenyl | RGD |
| Compound (Re-III-12c-RGD) | ¹⁸⁸Re | 4-(MeO(CH₂CH₂O)₃)phenyl | RGD |
| Compound (Re-III-13-RGD) | ¹⁸⁸Re | 4-dimethylaminophenyl | RGD |
| Compound (Re-III-14-RGD) | ¹⁸⁸Re | o-methoxyphenyl | RGD |
| Compound (Re-III-1-PSMAt1) | ¹⁸⁸Re | phenyl | PSMAt1 |
| Compound (Re-III-2-PSMAt1) | ¹⁸⁸Re | p-tolyl | PSMAt1 |
| Compound (Re-III-3-PSMAt1) | ¹⁸⁸Re | m-tolyl | PSMAt1 |
| Compound (Re-III-4-PSMAt1) | ¹⁸⁸Re | o-tolyl | PSMAt1 |
| Compound (Re-III-5-PSMAt1) | ¹⁸⁸Re | 2,3-xylyl | PSMAt1 |
| Compound (Re-III-6-PSMAt1) | ¹⁸⁸Re | 2,4-xylyl | PSMAt1 |
| Compound (Re-III-7-PSMAt1) | ¹⁸⁸Re | 2,5-xylyl | PSMAt1 |
| Compound (Re-III-8-PSMAt1) | ¹⁸⁸Re | 2,6-xylyl | PSMAt1 |
| Compound (Re-III-9-PSMAt1) | ¹⁸⁸Re | 3,4-xylyl | PSMAt1 |
| Compound (Re-III-10-PSMAt1) | ¹⁸⁸Re | 3,5-xylyl | PSMAt1 |
| Compound (Re-III-11-PSMAt1) | ¹⁸⁸Re | p-methoxyphenyl | PSMAt1 |
| Compound (Re-III-12a-PSMAt1) | ¹⁸⁸Re | 4-(MeO(CH₂CH₂O))phenyl | PSMAt1 |
| Compound (Re-III-12b-PSMAt1) | ¹⁸⁸Re | 4-(MeO(CH₂CH₂O)₂)phenyl | PSMAt1 |
| Compound (Re-III-12c-PSMAt1) | ¹⁸⁸Re | 4-(MeO(CH₂CH₂O)₃)phenyl | PSMAt1 |
| Compound (Re-III-13-PSMAt1) | ¹⁸⁸Re | 4-dimethylaminophenyl | PSMAt1 |
| Compound (Re-III-14-PSMAt1) | ¹⁸⁸Re | o-methoxyphenyl | PSMAt1 |
| Compound (186Re-III-2-RGD) | ¹⁸⁶Re | p-tolyl | RGD |
| Compound (186Re-III-3-RGD) | ¹⁸⁶Re | m-tolyl | RGD |
| Compound (186Re-III-4-RGD) | ¹⁸⁶Re | o-tolyl | RGD |
| Compound (186Re-III-5-RGD) | ¹⁸⁶Re | 2,3-xylyl | RGD |
| Compound (186Re-III-6-RGD) | ¹⁸⁶Re | 2,4-xylyl | RGD |
| Compound (186Re-III-7-RGD) | ¹⁸⁶Re | 2,5-xylyl | RGD |
| Compound (186Re-III-8-RGD) | ¹⁸⁶Re | 2,6-xylyl | RGD |
| Compound (186Re-III-9-RGD) | ¹⁸⁶Re | 3,4-xylyl | RGD |
| Compound (186Re-III-10-RGD) | ¹⁸⁶Re | 3,5-xylyl | RGD |
| Compound (186Re-III-11-RGD) | ¹⁸⁶Re | p-methoxyphenyl | RGD |
| Compound (186Re-III-12a-RGD) | ¹⁸⁶Re | 4-(MeO(CH₂CH₂O))phenyl | RGD |
| Compound (186Re-III-12b-RGD) | ¹⁸⁶Re | 4-(MeO(CH₂CH₂O)₂)phenyl | RGD |
| Compound (186Re-III-12c-RGD) | ¹⁸⁶Re | 4-(MeO(CH₂CH₂O)₃)phenyl | RGD |
| Compound (186Re-III-13-RGD) | ¹⁸⁶Re | 4-dimethylaminophenyl | RGD |
| Compound (186Re-III-14-RGD) | ¹⁸⁶Re | o-methoxyphenyl | RGD |
| Compound (186Re-III-1-PSMAt1) | ¹⁸⁶Re | phenyl | PSMAt1 |
| Compound (186Re-III-2-PSMAt1) | ¹⁸⁶Re | p-tolyl | PSMAt1 |
| Compound (186Re-III-3-PSMAt1) | ¹⁸⁶Re | m-tolyl | PSMAt1 |
| Compound (186Re-III-4-PSMAt1) | ¹⁸⁶Re | o-tolyl | PSMAt1 |
| Compound (186Re-III-5-PSMAt1) | ¹⁸⁶Re | 2,3-xylyl | PSMAt1 |
| Compound (186Re-III-6-PSMAt1) | ¹⁸⁶Re | 2,4-xylyl | PSMAt1 |
| Compound (186Re-III-7-PSMAt1) | ¹⁸⁶Re | 2,5-xylyl | PSMAt1 |
| Compound (186Re-III-8-PSMAt1) | ¹⁸⁶Re | 2,6-xylyl | PSMAt1 |
| Compound (186Re-III-9-PSMAt1) | ¹⁸⁶Re | 3,4-xylyl | PSMAt1 |
| Compound (186Re-III-10-PSMAt1) | ¹⁸⁶Re | 3,5-xylyl | PSMAt1 |
| Compound (186Re-III-11-PSMAt1) | ¹⁸⁶Re | p-methoxyphenyl | PSMAt1 |
| Compound (186Re-III-12a-PSMAt1) | ¹⁸⁶Re | 4-(MeO(CH₂CH₂O))phenyl | PSMAt1 |
| Compound (186Re-III-12b-PSMAt1) | ¹⁸⁶Re | 4-(MeO(CH₂CH₂O)₂)phenyl | PSMAt1 |
| Compound (186Re-III-12c-PSMAt1) | ¹⁸⁶Re | 4-(MeO(CH₂CH₂O)₃)phenyl | PSMAt1 |
| Compound (186Re-III-13-PSMAt1) | ¹⁸⁶Re | 4-dimethylaminophenyl | PSMAt1 |
| Compound (186Re-III-14-PSMAt1) | ¹⁸⁶Re | o-methoxyphenyl | PSMAt1 |

In some cases, there is provided a complex according to (c) Formula (Cu-IIIc-A) or Formula (Cu-IIIc-B) or a mixture thereof, or (d) according to Formula (Cu-IIId-A) or Formula (Cu-IIId-B) or a mixture thereof; wherein X and LIG are according to a line in the following table;

TABLE 4

| Compound | X | LIG |
|---|---|---|
| Compound (Cu-III-2-RGD) | p-tolyl | RGD |
| Compound (Cu-III-3-RGD) | m-tolyl | RGD |
| Compound (Cu-III-4-RGD) | o-tolyl | RGD |
| Compound (Cu-III-5-RGD) | 2,3-xylyl | RGD |
| Compound (Cu-III-6-RGD) | 2,4-xylyl | RGD |
| Compound (Cu-III-7-RGD) | 2,5-xylyl | RGD |
| Compound (Cu-III-8-RGD) | 2,6-xylyl | RGD |
| Compound (Cu-III-9-RGD) | 3,4-xylyl | RGD |
| Compound (Cu-III-10-RGD) | 3,5-xylyl | RGD |
| Compound (Cu-III-11-RGD) | p-methoxyphenyl | RGD |
| Compound (Cu-III-12a-RGD) | 4-(MeO(CH₂CH₂O))phenyl | RGD |
| Compound (Cu-III-12b-RGD) | 4-(MeO(CH₂CH₂O)₂)phenyl | RGD |
| Compound (Cu-III-12c-RGD) | 4-(MeO(CH₂CH₂O)₃)phenyl | RGD |
| Compound (Cu-III-13-RGD) | 4-dimethylaminophenyl | RGD |
| Compound (Cu-III-14-RGD) | o-methoxyphenyl | RGD |
| Compound (Cu-III-1-PSMAt1) | phenyl | PSMAt1 |
| Compound (Cu-III-2-PSMAt1) | p-tolyl | PSMAt1 |
| Compound (Cu-III-3-PSMAt1) | m-tolyl | PSMAt1 |
| Compound (Cu-III-4-PSMAt1) | o-tolyl | PSMAt1 |
| Compound (Cu-III-5-PSMAt1) | 2,3-xylyl | PSMAt1 |
| Compound (Cu-III-6-PSMAt1) | 2,4-xylyl | PSMAt1 |
| Compound (Cu-III-7-PSMAt1) | 2,5-xylyl | PSMAt1 |
| Compound (Cu-III-8-PSMAt1) | 2,6-xylyl | PSMAt1 |
| Compound (Cu-III-9-PSMAt1) | 3,4-xylyl | PSMAt1 |
| Compound (Cu-III-10-PSMAt1) | 3,5-xylyl | PSMAt1 |
| Compound (Cu-III-11-PSMAt1) | p-methoxyphenyl | PSMAt1 |
| Compound (Cu-III-12a-PSMAt1) | 4-(MeO(CH₂CH₂O))phenyl | PSMAt1 |
| Compound (Cu-III-12b-PSMAt1) | 4-(MeO(CH₂CH₂O)₂)phenyl | PSMAt1 |
| Compound (Cu-III-12c-PSMAt1) | 4-(MeO(CH₂CH₂O)₃)phenyl | PSMAt1 |
| Compound (Cu-III-13-PSMAt1) | 4-dimethylaminophenyl | PSMAt1 |
| Compound (Cu-III-14-PSMAt1) | o-methoxyphenyl | PSMAt1 |

Method of making the Diphosphine Precursor Compound

In a fourth aspect there is provided a method of making a diphosphine precursor compound according to Formula (I) comprising a step of mixing HPX₁X₂ and dichloromaleic anhydride in the presence of a base, wherein X₁ and X₂ are each independently according to any of the definitions provided herein.

This approach is different to the prior art in which dichloromaleic anhydride is added to diphenyl(trimethylsilyl)phosphine. One advantage is the improved atom economy because the present method dispenses with the presence of trimethylsilyl groups. Preferably, the dichloromaleic anhydride is added to the HPX₁X₂. The addition of the dichloromaleic anhydride to the HPX₁X₂ is preferably dropwise.

The base may be an organic base, such as an amine base, for example triethylamine. The organic base is preferably added dropwise. The reaction is preferably conducted in an organic solvent, such as diethyl ether. The reaction is preferably conducted at room temperature.

### Method of Making the Conjugated Diphosphine Precursor Compound

In a fifth aspect there is provided a method of making a conjugated diphosphine precursor compound according to Formula (II) comprising a step of mixing a compound of Formula (I) and LIG-H in the presence of a base, wherein LIG is according to any of the definitions provided herein.

The base may be an organic base, such as an amine base, for example N,N-diisopropylethylamine. The organic base may be added dropwise. The reaction is preferably conducted in an organic solvent, such as a protic polar solvent, for example N,N-dimethylformamide. The reaction is preferably conducted at room temperature.

Method of making the Radiolabelled Conjugated Diphosphine Complex

In a sixth aspect there is provided a method of making the radiolabelled conjugated diphosphine complex according to the third aspect, comprising the step of mixing a compound according to Formula (II) with a radionuclide, in the presence of an intermediate ligand, a reducing agent, a buffer and a solvent.

The radionuclide may be selected from one or more of ^{99m}Tc, ²¹²Bi, ²¹³Bi, ¹⁸⁶Re, ¹⁸⁸Re, ⁸⁹Zr, ⁶⁷Ga, ⁶⁸Ga, ⁶⁷Cu, ^{I}Cu, ⁶²Cu, ⁶¹Cu, ⁶⁰Cu and ⁵²Mn. Preferably the radionuclide is selected from ^{99m}Tc, ¹⁸⁶Re or ¹⁸⁸Re. The radionuclide may also be selected from ⁶⁷Cu, ⁶⁴Cu, ⁶²Cu, ⁶¹Cu and ⁶⁰Cu. The intermediate ligand is preferably a multidentate organic ligand, such as sodium tartrate. The reducing agent is preferably a metal salt, such as tin(II) chloride (dihydrate). The buffer is preferably a bicarbonate, such as sodium hydrogen carbonate. The solvent is preferably selected from one or more of water, a saline solution, methanol, ethanol, propanol and isopropanol.

### Kit Comprising the Conjugated Diphosphine Precursor Compound

In a seventh aspect there is provided a kit for preparing the radiolabelled conjugated diphosphine compound according to the third aspect comprising a mixture of a reducing agent, a buffering agent, an intermediate co-ligand and a conjugated diphosphine precursor compound of Formula (II).

The reducing agent may be a metal reducing agent, such as tin(II) chloride. There may be 0.2 to 2 equivalents, preferably 0.4 to 1.6 equivalents, more preferably 0.6 to 1.2 equivalents, of reducing agent relative to the conjugated diphosphine precursor compound.

The buffering agent may be an inorganic salt, such as sodium bicarbonate. There may be 10 to 400 equivalents, preferably 20 to 200 equivalents, more preferably 50 to 100 equivalents, of buffering agent relative to the conjugated diphosphine precursor compound.

The intermediate co-ligand may be a bidentate organic ligand, such as sodium tartrate or potassium tartrate. There may be 0.2 to 2 equivalents, preferably 0.4 to 1.6 equivalents, more preferably 0.6 to 1.2 equivalents, of intermediate co-ligand relative to the conjugated diphosphine precursor compound. Alternatively, there may be 1 to 40 equivalents, preferably 10 to 40 equivalents, more preferably 20 to 40 equivalents of bidentate organic ligand relative to the conjugated diphosphine precursor compound.

In some instances, the kit for preparing the radiolabelled conjugated diphosphine compound comprises a mixture of 0.2 to 2 equivalents of reducing agent, 10 to 400 equivalents of a buffering agent, 0.2 to 2 equivalents of an intermediate co-ligand and 1 equivalent of a conjugated diphosphine precursor compound of Formula (II).

In some instances, the kit for preparing the radiolabelled conjugated diphosphine compound comprises a mixture of 0.2 to 2 equivalents of reducing agent, 10 to 400 equivalents of a buffering agent, 20 to 30 equivalents of an intermediate co-ligand and 1 equivalent of a conjugated diphosphine precursor compound of Formula (II).

In some cases, the kit comprises
(i) (II-1-RGD) or (II-2-RGD): 1 mg (0.93 μmol); sodium gluconate (NaC₆H₁₁O₇): 1 mg (4.6 μmol); SnCl₂·2H₂O: 50 μg (0.22 μmol) and NaHCO₃: 1.8 mg (21.4 μmol); or
(ii) (II-1-RGD) or (II-2-RGD): 500 μg (0.47 μmol); sodium tartrate (Na₂C₄H₄06): 1.05 mg (4.6 μmol); SnCl₂·2H₂O: 50 μg (0.22 μmol) and NaHCO₃: 1.8 mg (21.4 mol); or
(iii) (II-1-RGD) or (II-2-RGD): 125 g (0.12 μmol); sodium tartrate: 0.26 mg (1.15 mol); SnCl₂·2H₂O: 25 μg (0.11 μmol) and NaHCO₃: 0.9 mg (10.7 μmol); or
(iv) (II-1-RGD) or (II-2-RGD): 63 μg (0.06 μmol); sodium tartrate: 0.26 mg (1.15 mol); SnCl₂·2H₂O: 25 μg (0.11 μmol); NaHCO₃: 0.9 mg (10.7 μmol); or
(v) (II-1-PSMAt1) or (II-2-PSMAt1) 110-120 μg, sodium tartrate: 0.26 mg (1.15 μmol); SnCl₂·2H₂O: 25 μg and NaHCO₃: 0.9 mg (10.7 μmol); or
(vi) (II-1-PSMAt1) 85 μg (0.08 μmol), sodium tartrate: 0.53 mg (2.29 μmol), SnCl₂·2H₂O: 19.0 μg (0.08 μmol), NaHCO₃: 0.90 mg (10.71 μmol).

The kits may be used by adding a mixture of saline and ethanol to dissolve the conjugated diphosphine precursor compound; lower amounts of ethanol were required for kits containing lower amounts of the conjugated diphosphine precursor compound. In some cases, aqueous saline solution is used without ethanol. In some cases, more than one kit is used.

In some cases, the kit mixture is a lyophilised mixture. The kits may be stored at 0 to 4° C. prior to use. In some instances, it is preferable that the kit is stored at about −18° C. prior to use. The kit may provide radiochemical yields of about 85% or more, such as about 90% or more or about 95% or more.

In some cases, the kit comprises a radionuclide selected from ^{99m}Tc, ²¹²Bi, ²¹³Bi, ¹⁸⁶Re, ¹⁸⁸Re, ⁸⁹Zr, ⁶⁷Ga, ⁶⁸Ga, ⁶⁷Cu, 64Cu, ⁶²Cu, ⁶¹Cu, ⁶⁰CU and ⁵²Mn. The radionuclide is preferably ^{99m}Tc and/or ¹⁸⁸Re.

### Uses and Methods

In another aspect there is provided use of a diphosphine precursor compound according to Formula (I), a conjugated diphosphine precursor compound according to Formula (II) or a radiolabelled conjugated diphosphine complex according to the third aspect in the preparation of a medicament for the treatment or diagnosis of a disease.

In another aspect there is provided a diphosphine precursor compound according to Formula (I), a conjugated diphosphine precursor compound according to Formula (II) or a radiolabelled conjugated diphosphine complex according to the third aspect for use in the treatment or diagnosis of a disease. One such use is in imaging studies.

In another aspect there is provided an in vivo method of imaging a tumour, comprising administering a radiolabelled conjugated diphosphine complex according to the third aspect to a subject and detecting the radionuclide. In another aspect there is provided a method of treating or diagnosing a disease comprising administering a radiolabelled conjugated diphosphine complex according to the third aspect to a subject.

The disease may be one or more of cancer (breast cancer, lung cancer, prostate cancer, myeloma, melanoma, ovarian cancer, thyroid cancer, kidney cancer, pancreatic cancer, neuroendocrine cancer or head and neck cancer), an autoimmune disease (systemic lupus erythematosus, rheumatoid arthritis, Sjögren's syndrome, graft-versus-host-disease, and myasthenia gravis; chronic inflammatory conditions such as psoriasis, asthma and Crohn's disease) or an inflammatory disease (vasculitis, in particular Kawasaki disease, cystic fibrosis, chronic inflammatory intestinal diseases such as ulcerative colitis or Crohn's disease, chronic bronchitis, inflammatory arthritis diseases such as psoriatic arthritis, rheumatoid arthritis, and systemic onset juvenile rheumatoid arthritis (SOJRA, Still's disease)) and bone metastases.

In another aspect there is provided use of a diphosphine precursor compound according to Formula (I), a conjugated diphosphine precursor compound according to Formula (II) or a radiolabelled conjugated diphosphine precursor complex according to the third aspect in imaging or cell labelling, optionally wherein the use is non-therapeutic and/or in vitro. Preferably, there is provided use of said compounds in SPECT (single-photon emission computed tomography) or gamma-scintigraphy. Even more preferably, there is provided use of said compounds in PET (positron emission spectroscopy) where the radionuclide is ⁶⁴Cu or MRT (molecular radiotherapy) where the radionuclide is ¹⁸⁸Re or ¹⁸⁶Re.

The disclaimers applied to each of Formula (I), Formula (II) and/or the third aspect herein may be applied to all aspects of the invention, such as the kits, uses, medical uses and methods. That is to say, any of Compound (I-1), Compound (II-1-RGD), Compound (III-1-RGD) and Compound (Re-1-RGD) may be independently included or excluded from any aspect herein.

### GENERAL DEFINITIONS

For any general chemical formula herein, it is to be understood that any of the variable group definitions provided herein, such as A, Y, Z, X, X₁, X₂, X₃, X₄, R₁, R₂, R₃ and R₄, including those shown in specific examples, may be applied in combination with any of the other variable group definitions. All possible combinations of the variable group definitions with each general formula are therefore disclosed and may be claimed.

### SUMMARY OF THE FIGURES

So that the invention may be understood, and so that further aspects and features thereof may be appreciated, embodiments illustrating the principles of the invention will now be discussed in further detail with reference to the accompanying figures, in which:

FIG. 1A shows (Tc-III-1-RGD) (i.e. [^{99m}TcO₂(II-1-RGD)₂]⁺) binding to αvβ3 integrin receptor, which can be inhibited by increasing concentrations of the peptide (RGD).

FIG. 1B shows the biodistribution of (Tc-III-1-RGD) in healthy mice at 1 hour post injection (left bars): co-injection of 400 g peptide inhibits (Tc-III-1-RGD) uptake in αvβ3 integrin-expressing tissue (right bars). Error bars correspond to 95% confidence interval.

FIG. 1C shows that in mice with rheumatoid arthritis, (Tc-III-1-RGD) accumulation in ankles (crosses) and wrists (triangles) correlates with joint swelling.

FIG. 1D shows a maximum intensity projection of a SPECT/CT image of a mouse with rheumatoid arthritis, showing accumulation of (Tc-III-1-RGD) in an arthritic ankle (RA). Bl=bladder, K=kidneys, Th=thyroid.

FIG. 2A shows a ³¹P{H}NMR of Compound (II-1-RGD), cis-(^{nat}Re-III-1-RGD) and trans-(^{nat}Re-III-1-RGD);

FIG. 2B shows a radio-HPLC trace of trans-/cis-(Tc-III-1-RGD) (upper line) prepared from an aqueous solution of ^{99m}TcO₄⁻ and Kit 3 (Table 7), and HPLC traces (λ₂₂₀) of cis-(^{nat}Re-III-1-RGD) (dashed lower line) and trans-(^{nat}Re-III-1-RGD) (solid lower line).

FIG. 3 shows stability in serum. (Tc-III-1-RGD) was incubated in human serum for 4 h. C18 Analytical radio-HPLC analysis revealed that 0.5% ^{99m}Tc dissociated from (Tc-III-1-RGD) over 1 h, and 3% ^{99m}Tc dissociated from (Tc-III-1-RGD) over 4 h.

FIG. 4 shows the quantification of radioactivity distribution in the urine of the bladder (down triangles), kidneys (up triangles), liver (squares) and heart/blood pool (circles) from SPECT/CT imaging of a single healthy Balb/c mouse administered (Tc-III-1-RGD) intravenously.

FIG. 5 shows an analytical reverse phase C18 UV (254 nm) HPLC trace of Compound (II-1-RGD).

FIG. 6 shows a radio-HPLC analysis of urine from a healthy Balb/c mouse that was intravenously administered (Tc-III-1-RGD) which shows that it is excreted intact.

FIG. 7 shows that in mice with induced rheumatoid arthritis, administered (Tc-III-1-RGD), radioactivity concentration in ankles (crosses) and wrists (triangles) (measured using SPECT/CT image quantification) correlates with degree of joint swelling (measured using calipers). For ankles, y=1.89x+1.587, R²=0.69, and p=0.04 (significance of slope from non-zero). For wrists, y=1.01*x+1.09, R² and p=0.12.

FIG. 8 shows the biodistribution of (Tc-III-1-RGD) in rheumatoid arthritis mice 1 hour post-injection (n=3). Error bars correspond to standard deviation.

FIG. 9 shows the full ³¹P{H}NMR of Compound (II-1-RGD) (top), cis-(Re-III-1-RGD) (middle) and trans-(Re-III-1-RGD) (bottom).

FIG. 10 shows SPECT/CT maximum intensity projection images of a balb/c mouse administered (Tc-III-1-RGD) intravenously. SPECT images were acquired over 4 hours in 30 min segments. Imaging analysis indicated that the majority of (Tc-III-1-RGD) cleared rapidly via a renal pathway: at 30 min PI (post-injection), 35% of the injected dose of radioactivity was in the bladder; at 2 h PI, 56% was in the bladder.

FIG. 11 shows that geometric isomer 1 and geometric isomer 2, one of which corresponds to the “cis” geometric isomer and the other of which corresponds to the “trans” geometric isomers of (Tc-III-1-PSMAt1): both have near identical uptake in PSMA-positive cells.

FIG. 12 shows in vitro uptake of (Tc-III-1-PSMAt1) and (Tc-III-2-PSMAt1) uptake following 60 min incubation in PSMA-positive (DU145-PSMA and LNCAP) and PSMA-negative cell lines (DU-145 and PC-3). From left to right (for each complex), the bars represent DU145-PSMA+, DU145-PSMA+ with PMPA, DU145, LNCAP, LNCAP with PMPA, and PC-3. Uptake was blocked with the PSMA inhibitor 2-phosphonomethyl pentanedioic acid (PMPA). Scatter plots represent biological repeats performed in triplicate.

FIG. 13 shows SPECT images of healthy mice from 15 min-4 hours post-injection (intravenous) of (Tc-III-1-PSMAt1) (top) and (Tc-III-2-PSMAt1) (bottom). This shows that (i) both (Tc-III-1-PSMAt1) and (Tc-III-2-PSMAt1) clear circulation via a renal pathway (this is ultimately favourable for cancer imaging), and (ii) (Tc-III-1-PSMAt1) clears kidneys faster than (Tc-III-2-PSMAt1).

FIG. 14 shows ex vivo biodistribution of healthy mice 2 h post-injection of either (Tc-III-1-PSMAt1) or (Tc-III-2-PSMAt1) (a) all harvested/dissected organs and tissue except kidneys and (b) kidneys. Notably, higher amounts of (Tc-III-2-PSMAt1) are present in kidneys 2 h post-injection compared to (Tc-III-1-PSMAt1). There are also significant amounts of both tracers accumulated in the spleen, salivary glands and prostate—tissues that are known to express PSMA or take up PSMA-targeted compounds. Uptake of (Tc-III-1-PSMAt1) in the spleen and salivary glands is significantly higher than uptake of (Tc-III-2-PSMAt1) in these organs.

FIG. 15 shows analytical reverse-phase radio-HPLC chromatograms of urine collected from mice administered either (a) (Tc-III-1-PSMAt1) or (b) (Tc-III-2-PSMAt1). The retention time of each radioactive peak matches that of either (Tc-III-1-PSMAt1) or (Tc-III-2-PSMAt1), indicating that each ^{99m}Tc-radiotracer is excreted intact and has high metabolic stability. Analytical HPLC conditions: 20 min, 5% min⁻¹ linear increase from 100% A to 100% B (flow rate of 1 ml/min, A=water containing 0.1% TFA (trifluoroacetic acid), B=acetonitrile containing 0.1% TFA, analytical (4.6×150 mm, 5 μm) Agilent Zorbax Eclipse XDB-C18 column).

FIG. 16 shows analytical reverse-phase radio-HPLC chromatograms of (Tc-III-1-PSMAt1) and (Tc-III-2-PSMAt1) after kit-based radiolabelling reactions undertaken either at 5 min at room temperature or 100° C. Analytical HPLC conditions: 20 min, 5% min⁻¹ linear increase from 100% A to 100% B (flow rate of 1 ml/min, A=water containing 0.1% TFA, B=acetonitrile containing 0.1% TFA, analytical (4.6×150 mm, 5 μm) Agilent Zorbax Eclipse XDB-C18 column).

FIG. 17 Biodistribution of SCID/Beige mice bearing either DU145-PSMA+ or DU145 prostate cancer tumours. Either (Tc-III-1-PSMAt1) or (Tc-III-2-PSMAt1) were administered to mice intravenously. To assess the specificity of radiotracer uptake, three experimental groups of mice (n=5 per group) were used. In the first group, mice bearing DU145-PSMA+ prostate cancer tumours were administered either (Tc-III-1-PSMAt1) or (Tc-III-2-PSMAt1). In the second group, mice bearing DU145-PSMA+ prostate cancer tumours were co-administered 2-phosphonomethyl pentanedioic acid (PMPA) (to inhibit radiotracer PSMA receptor uptake) and either (Tc-III-1-PSMAt1) or (Tc-III-2-PSMAt1). In the third group, mice bearing DU145 prostate cancer tumours, which do not express PSMA receptor, were administered either (Tc-III-1-PSMAt1) or (Tc-III-2-PSMAt1). All animals were culled 2 h post-injection, and organs were harvested, weighed and counted for radioactivity. Separately, mice bearing DU145-PSMA+ prostate cancer tumours were administered either (Tc-III-1-PSMAt1) or (Tc-III-2-PSMAt1) (n=3 per group), and culled 24 h post-injection. (a) Tumour uptake/retention of radiotracers; (b) Kidney uptake/retention of radiotracers; (c) Biodistribution of (Tc-III-1-PSMAt1) in organs/tissues excluding tumours and kidneys; (d) Biodistribution of (Tc-III-2-PSMAt1) in organs/tissues excluding tumours and kidneys. Error bars correspond to standard deviation. In (a) and (b) the far left bars are “tracer, DU145-PSMA+(2 h)”, the left bars are “tracer, DU145-PSMA+(24 h)”, the right bars are “tracer+ PMPA, DU145-PSMA+(2 h)” and the far right bars are “tracer, DU145 (2 h)”. In (c) and (d) the order of the bars is the same except that “tracer, DU145-PSMA+(24 h)” is not present.

FIG. 18a shows a whole body SPECT/CT maximum intensity projection of SCID/Beige mice bearing either DU145-PSMA+ tumours or DU145 tumours, administered either (Tc-III-1-PSMAt1) or (Tc-III-2-PSMAt1), 2 h post-injection. To inhibit uptake in DU145-PSMA+ tumours, animals were also administered PMPA.

FIG. 18b shows a whole-body SPECT/CT maximum intensity projection of SCID/Beige mice bearing DU145-PSMA+ tumours 24 h post-injection, administered either (Tc-III-1-PSMAt1) or (Tc-III-2-PSMAt1).

FIG. 19 shows Reverse phase radio-HPLC trace of (a) (Re-III-1-PSMAt1), (b) (Re-III-2-PSMAt1) and (c) (Re-III-11-PSMAt1). Analytical HPLC conditions: 30 min linear increase from 100% A to 100% B (flow rate of 1 ml/min, A=water containing 0.1% TFA, B=acetonitrile containing 0.1% TFA, analytical (4.6×150 mm, 5 μm) Agilent Zorbax Eclipse XDB-C18 column).

FIG. 20 shows stability of (Tc-III-11-PSMAt1) in serum. (Tc-III-11-PSMAt1) was incubated in human serum for 24 h. C18 Analytical radio-HPLC analysis revealed that >95% of (Tc-III-11-PSMAt1) remained intact after 24 h incubation. Analytical HPLC conditions: 20 min linear increase from 100% A to 100% B (flow rate of 1 ml/min, A=water containing 0.1% TFA, B=acetonitrile containing 0.1% TFA, analytical (4.6×150 mm, 5 μm) Agilent Zorbax Eclipse XDB-C18 column).

FIG. 21 shows time course uptake and localisation of i) (Tc-III-1-PSMAt1) and ii) (Tc-III-2-PSMAt1) in (a) DU145-PSMA cells and (b) LNCaP cells. Data are presented as mean±SD, n=3 biological repeats performed in triplicate.

FIG. 22 shows reverse phase radio-HPLC chromatograms showing the stability of a) (Re-III-1-PSMAt1) and b) (Re-II-2-PSMAt1). Both (Re-III-1-PSMAt1) and (Re-II-2-PSMAt1) are stable for up to 24 h after incubation in human serum at 37° C.

FIG. 23 shows in vitro uptake of (Re-III-1-PSMAt1) and (Re-III-2-PSMAt1). (Re-III-1-PSMAt1) and (Re-III-2-PSMAt1) uptake following 60 min incubation in PSMA-positive (DU145-PSMA+) and PSMA-negative (DU-145) cell lines. Uptake was blocked with the PSMA inhibitor PMPA. Scatter plots represent biological repeats performed in triplicate. *, p<0.05 **, p<0.01; ***, p<0.001, ****, p<0.0001.

FIG. 24 shows uptake of (¹⁸⁶Re-III-1-PSMAt1) in PSMA-expressing DU145-PSMA+ and LNCaP prostate cancer cells, and PSMA-negative DU145 prostate cancer cells. (¹⁸⁶Re-III-1-PSMAt1) was also co-incubated with an excess of the PSMA inhibitor, PMPA. *, p<0.05 **, p<0.01; ***, p<0.001, ****, p<0.0001; n=2-5. Data are presented as mean±SD.

FIG. 25 shows ex vivo biodistribution of mice 2 h post-injection (n=4 per group) of either (¹⁸⁸Re-III-1-PSMAt1) or (¹⁸⁸Re-III-2-PSMAt1): (a) All harvested/dissected organs and tissue except kidneys and (b) Kidneys

FIG. 26 shows reverse-phase radio-HPLC chromatograms of (a-i) (¹⁸⁸Re-III-1-PSMAt1); (a-ii) urine collected from a mouse 2 hours post-administration of (¹⁸⁸Re-III-1-PSMAt1); (b-i) (¹⁸⁸Re-III-2-PSMAt1; and (b-ii) urine collected from a mouse 2 hours post-administration of (¹⁸⁸Re-III-2-PSMAt1). Analytical HPLC conditions: 30 min, linear increase from 100% A to 100% B (flow rate of 1 ml/min, A =water containing 0.1% TFA, B=acetonitrile containing 0.1% TFA, analytical (4.6×150 mm, 5 μm) Agilent Zorbax Eclipse XDB-C18 column).

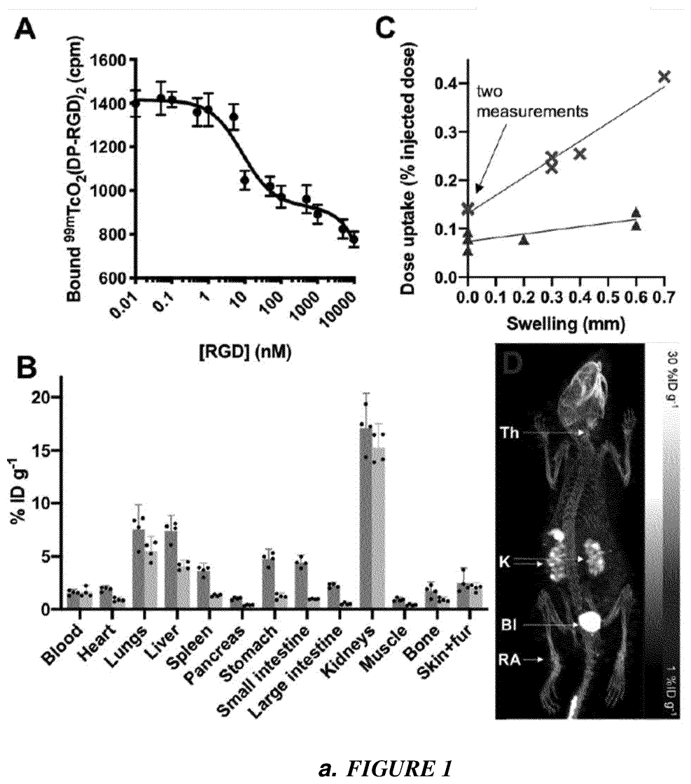

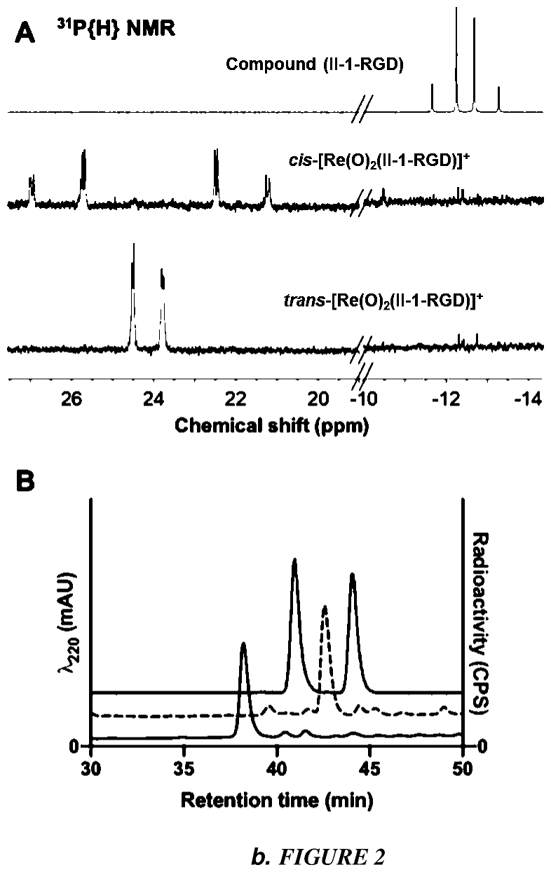

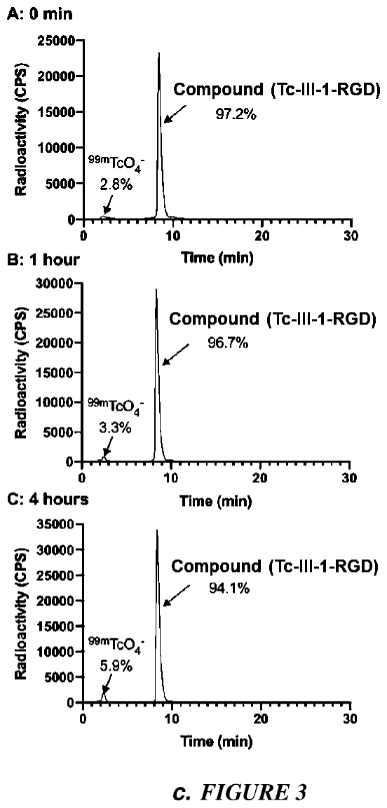

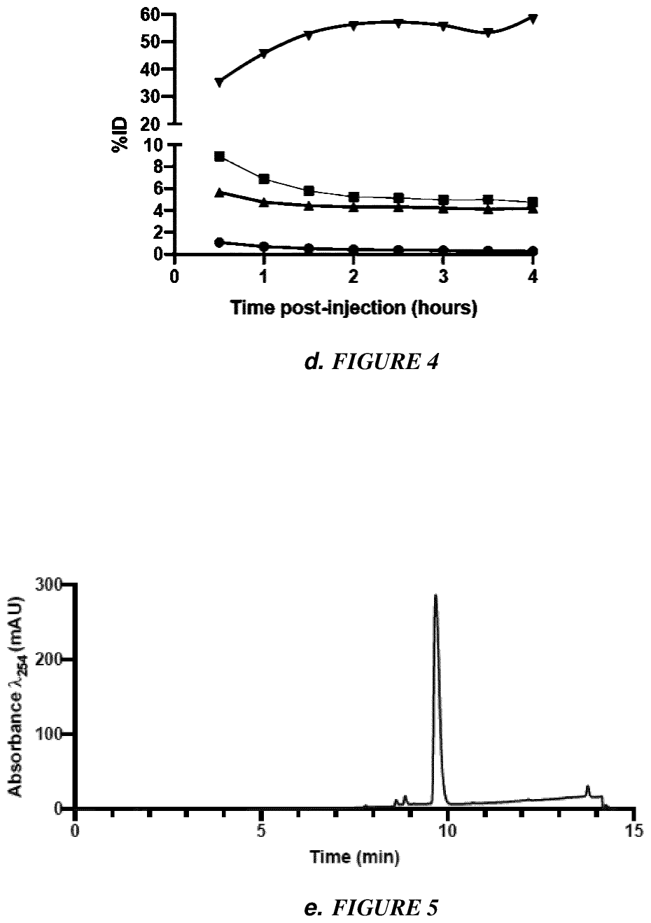

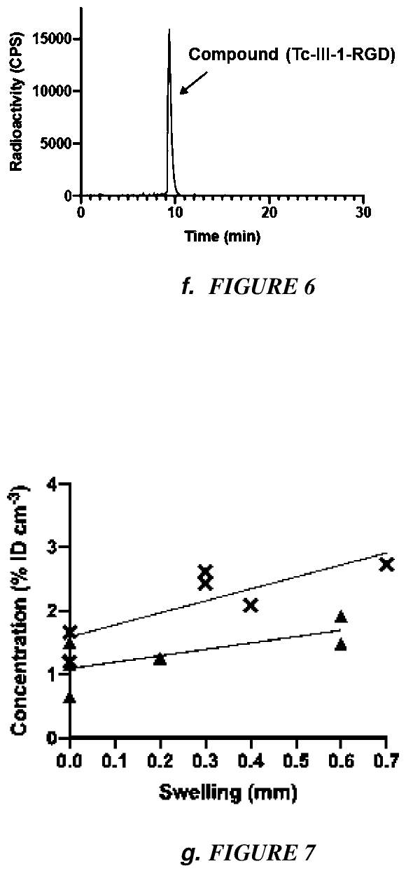

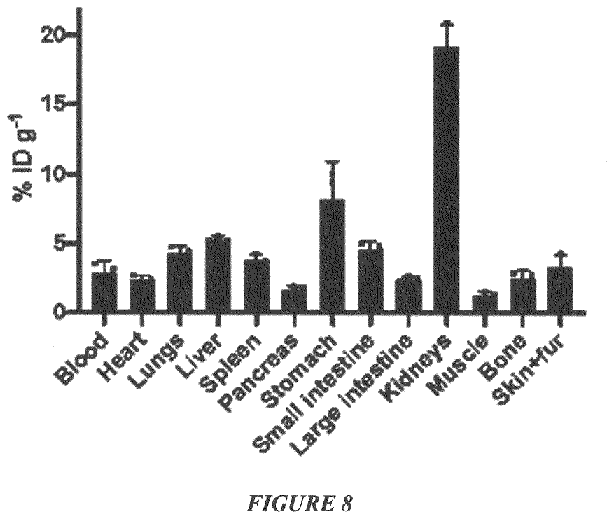

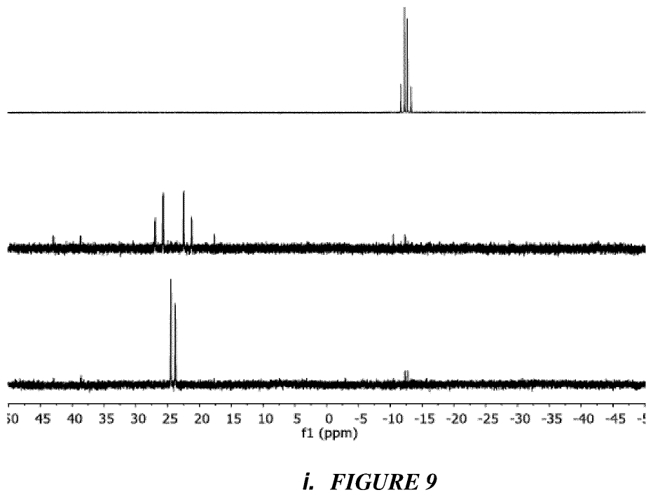

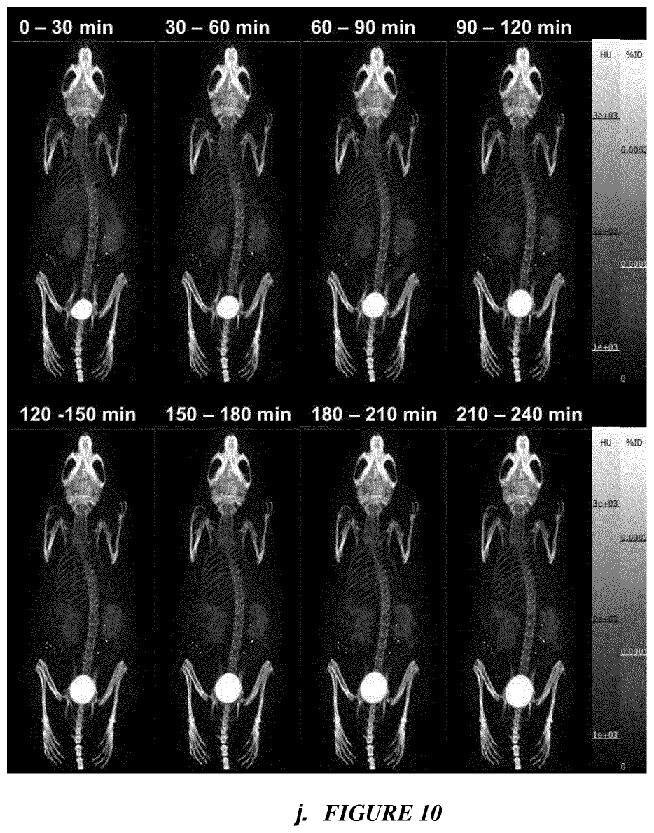

### DETAILED DESCRIPTION

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the words “have”, “comprise”, and “include”, and variations such as “having”, “comprises”, “comprising”, and “including” will be understood to imply the inclusion of a stated integer or step or group of integers or steps but not the exclusion of any other integer or step or group of integers or steps. However, each disclosure herein also includes the option of excluding any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means, for example, +/−10%.

The words “preferred” and “preferably” are used herein refer to embodiments of the invention that may provide certain benefits under some circumstances. It is to be appreciated, however, that other embodiments may also be preferred under the same or different circumstances. The recitation of one or more preferred embodiments therefore does not mean or imply that other embodiments are not useful, and is not intended to exclude other embodiments from the scope of the disclosure, or from the scope of the claims.

The compounds of the present invention include isomers, salts, solvates, and chemically protected forms thereof, as explained in more detail below.

In the present invention, alkyl groups are generally C₁-C₄ alkyl groups. The term “C₁-C₄ alkyl”, as used herein, includes a monovalent moiety obtained by removing a hydrogen atom from a C₁-C₄ hydrocarbon compound having from 1 to 4 carbon atoms, which may be aliphatic or alicyclic, or a combination thereof, and which may be saturated, partially unsaturated, or fully unsaturated. The term “C₁-C₄ alkyl” includes methyl, ethyl, n-propyl, isopropyl, cyclopropyl, n-butyl, isobutyl, t-butyl, cyclobutyl, ethenyl, cis/trans-1-propenyl, 2-propenyl, cis/trans-1-butenyl, cis/trans-2-butenyl and 3-butenyl. In preferred embodiments, the C₁-C₄ alkyl group is a saturated alkyl group and/or an acyclic alkyl group. In even more preferred embodiments the C₁-C₄ alkyl group is methyl or an ethyl group as shorter chain alkyl groups tend to make the compounds of the present invention less hydrophobic.

In the present invention, alkoxy groups are generally C₁-C₄ alkoxy groups. The term “C₁-C₄ alkoxy”, as used herein, includes a monovalent moiety obtained by removing the hydrogen atom from the oxygen atom of a C₁-C₄ alcohol compound having from 1 to 4 carbon atoms, which may be aliphatic or alicyclic, or a combination thereof, and which may be saturated, partially unsaturated, or fully unsaturated. The term “C₁-C₄ alkoxy” includes methoxy, ethoxy, n-propoxy, isopropoxy, cyclopropoxy, n-butoxy, isobutoxy, t-butoxy, cyclobutoxy, ethenoxy, cis/trans-1-propenoxy, 2-propenoxy, cis/trans-1-butenoxy, cis/trans-2-butenoxy and 3-butenoxy. In preferred embodiments, the C₁-C₄ alkoxy group is a saturated alkoxy group and/or an acyclic alkoxy group. In even more preferred embodiments the C_{I}-C₄ alkoxy group is methoxy or an ethoxy group as shorter chain alkoxy groups tend to make the compounds of the present invention less hydrophobic.

In the present invention, a “heteroaryl group” is generally a C₅-C₁₂ heteroaryl group, and is preferably a 5 or 6 membered heteroaryl group and as used herein refers to a monovalent moiety obtained by removing a hydrogen atom from a ring atom of a C₃-C₁₂ heterocyclic compound. The heteroaryl groups may be partially or fully unsaturated. The present invention provides example of compounds in which one or more pyridyl groups (e.g. one or more 2-pyridyl groups) are present. However, examples of heteroaryl compounds that could be employed in accordance with the present invention include:

Imidazole: a five membered aromatic ring having two nitrogen atoms and three carbon atom.

Triazole: a five membered aromatic ring having three nitrogen atoms and two carbon atoms, with two ring isomers 1,2,3,triazole, 1,2,4 triazole.

Tetrazole: a five membered aromatic ring having four nitrogen atoms and one carbon atom.

Pyridine: a six membered aromatic ring having one nitrogen atom and 5 carbon atoms.

Diazine: a six membered aromatic ring having two nitrogen atoms and four carbon atoms, with three ring isomers, 1,2-diazine, 1,3-diazine and 1,4-diazine.

Triazine: a six membered aromatic ring having three nitrogen atoms and three carbon atoms, with three ring isomers, 1,2,3-triazine, 1,2,4-triazine and 1,3,5-triazine.

Tetrazine: a six membered aromatic ring having four nitrogen atoms and two carbon atoms, with three ring isomers 1,2,3,4-tetrazine, 1,2,3,5-tetrazine and 1,2,4,5-tetrazine.

Fused ring systems such as quinoline, isoquinoline and indole.

It is generally preferred that the sp² nitrogen containing heterocyclic group has a donor electron pair in the ortho position relative to the methylene bridge of the bisphosphonate compound in order to facilitate chelation of the radionuclide by the heteroatom. A preferred heteroatom is nitrogen, i.e. providing pyridyl heteroaryl groups.

In the present invention, “Re” and “¹⁸⁸Re” refer to rhenium-188 (¹⁸⁸Re), whereas “¹⁸⁶Re” refers to rhenium-186, and “^{nat}Re” refers to naturally abundant rhenium. “Tc” and “^{99m}Tc” refer to technetium-99m (^{99m}Tc), whereas “^{99g}Tc” refers to techniutium-99g. “^{nat}Cu” refers to naturally abundant copper.

### Other Forms of the Substituents

Included in the above are the well-known ionic, salt, solvate, and protected forms of these substituents. For example, a reference to carboxylic acid (-COOH) also includes the anionic (carboxylate) form (—COO—), a salt or solvate thereof, as well as conventional protected forms. Similarly, a reference to an amino group includes the protonated form (N⁺HR¹R²), a salt or solvate of the amino group, for example, a hydrochloride salt, as well as conventional protected forms of an amino group. Similarly, a reference to a hydroxyl group also includes the anionic form (—O—), a salt or solvate thereof, as well as conventional protected forms of a hydroxyl group.

### Isomers, Salts, Solvates, Protected Forms, and Prodrugs

Certain compounds may exist in one or more particular geometric, optical, enantiomeric, diasteriomeric, epimeric, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and 1-forms; (+) and (−) forms; keto, enol-, and enolate-forms; syn- and anti-forms; synclinal- and anticlinal-forms; α- and β-forms; axial and equatorial forms; boat-, chair-, twist-envelope-, and half chair-forms; and combinations thereof, hereinafter collectively referred to as “isomers” (or “isomeric forms”).

Note that, except as discussed below for tautomeric forms, specifically excluded from the term “isomers”, as used herein, are structural (or constitutional) isomers (i.e. isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures or to a general formula includes structurally isomeric forms falling within that class or formula and, except where specifically stated or indicated, all possible conformations and configurations of the compound(s) herein are intended to be included in the general formula(e).

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hyroxyazo, and nitro/aci-nitro.

Note that specifically included in the term “isomer” are compounds with one or more isotopic substitutions. For example, H may be in any isotopic form, including ¹H, ²H(D), and ³H(T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including (wholly or partially) racemic and other mixtures thereof. Methods for the preparation (e.g. asymmetric synthesis) and separation (e.g., fractional crystallisation and chromatographic means) of such isomeric forms are either known in the art or are readily obtained by adapting the methods taught herein, or known methods, in a known manner.

Unless otherwise specified, a reference to a particular compound also includes ionic, salt, solvate, and protected forms of thereof, for example, as discussed below.

It may be convenient or desirable to prepare, purify, and/or handle a corresponding salt of the active compound, for example, a pharmaceutically-acceptable salt. Examples of pharmaceutically acceptable salts are discussed in Berge, et al., J. Pharm. Sci., 66, 1-19 (1977).

For example, if the compound is anionic, or has a functional group which may be anionic (e.g., COOH may be COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)⁴⁺.

If the compound is cationic, or has a functional group which may be cationic (e.g., NH₂ may be NH³⁺), then a salt may be formed with a suitable anion. Examples of suitable inorganic anions include, but are not limited to, those derived from the following inorganic acids: hydrochloric, hydrobromic, hydroiodic, sulphuric, sulphurous, nitric, nitrous, phosphoric, and phosphorous. Examples of suitable organic anions include, but are not limited to, those derived from the following organic acids: acetic, propionic, succinic, glycolic, stearic, palmitic, lactic, malic, pamoic, tartaric, citric, gluconic, ascorbic, maleic, hydroxymaleic, phenylacetic, glutamic, aspartic, benzoic, cinnamic, pyruvic, salicyclic, sulfanilic, 2-acetyoxybenzoic, fumaric, phenylsulfonic, toluenesulfonic, methanesulfonic, ethanesulfonic, ethane disulfonic, oxalic, pantothenic, isethionic, valeric, lactobionic, and gluconic. Examples of suitable polymeric anions include, but are not limited to, those derived from the following polymeric acids: tannic acid, carboxymethyl cellulose.

It may be convenient or desirable to prepare, purify, and/or handle a corresponding solvate of the active compound. The term “solvate” is used herein in the conventional sense to refer to a complex of solute (e.g. active compound, salt of active compound) and solvent. If the solvent is water, the solvate may be conveniently referred to as a hydrate, for example, a mono-hydrate, a di-hydrate, a tri-hydrate, etc.

It may be convenient or desirable to prepare, purify, and/or handle the active compound in a chemically protected form. The term “chemically protected form”, as used herein, includes a compound in which one or more reactive functional groups are protected from undesirable chemical reactions, that is, are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, ‘Protective Groups in Organic Synthesis’ (T. Green and P. Wuts, Wiley, 1999).

For example, a hydroxy group may be protected as an ether (—OR) or an ester (—OC(═O)R), for example, as: a t-butyl ether; a benzyl, benzhydryl (diphenylmethyl), or trityl (triphenylmethyl) ether; a trimethylsilyl or t-butyldimethylsilyl ether; or an acetyl ester (—OC(═O)CH₃, —OAc).

For example, an aldehyde or ketone group may be protected as an acetal or ketal, respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid.

For example, an amine group may be protected, for example, as an amide or a urethane, for example, as: a methyl amide (—NHCO—CH₃); a benzyloxy amide (—NHCO—OCH₂C₆H₅, —NH-Cbz); as a t-butoxy amide (—NHCO—OC(CH₃)₃, —NH—Boc); a 2-biphenyl-2-propoxy amide (—NHCO—OC(CH₃)₂C₆H₄C₆H₅, —NH-Bpoc), as a 9-fluorenylmethoxy amide (—NH—Fmoc), as a 6-nitroveratryloxy amide (—NH—Nvoc), as a 2-trimethylsilylethyloxy amide (—NH—Teoc), as a 2,2,2-trichloroethyloxy amide (—NH-Troc), as an allyloxy amide (—NH—Alloc), as a 2(-phenylsulphonyl)ethyloxy amide (—NH—Psec); or, in suitable cases, as an N-oxide (>NO).

For example, a carboxylic acid group may be protected as an ester for example, as: an C₁-C₇ alkyl ester (e.g. a methyl ester; a t-butyl ester); a C₁-C₇ haloalkyl ester (e.g., a C₁-C₇-trihaloalkyl ester); a tri-C₁-C₇-alkylsilyl-C₁-C₇-alkyl ester; or a C₅-C₂₀ aryl-C₁-C₇-alkyl ester (e.g. a benzyl ester; a nitrobenzyl ester); or as an amide, for example, as a methyl amide.

It may be convenient or desirable to prepare, purify, and/or handle the active compound in the form of a prodrug. The term “prodrug”, as used herein, includes a compound which, when metabolised (e.g. in vivo), yields the desired active compound. Typically, the prodrug is inactive, or less active than the active compound, but may provide advantageous handling, administration, or metabolic properties.

For example, some prodrugs are esters of the active compound (e.g. a physiologically acceptable metabolically labile ester). During metabolism, the ester group (—C(═O)OR) is cleaved to yield the active drug. Such esters may be formed by esterification, for example, of any of the carboxylic acid groups (-C(═O)OH) in the parent compound, with, where appropriate, prior protection of any other reactive groups present in the parent compound, followed by deprotection if required. Examples of such metabolically labile esters include those wherein R is C₁₋₇ alkyl (e.g. —Me, -Et); C₁₋₇ aminoalkyl (e.g. aminoethyl; 2-(N,N-diethylamino)ethyl; 2-(4 morpholino)ethyl); and acyloxy-C₁-C₇ alkyl (e.g. acyloxymethyl; acyloxyethyl; e.g. pivaloyloxymethyl; acetoxymethyl; 1-acetoxyethyl; 1-(1-methoxy-1-methyl)ethyl-carbonxyloxyethyl; 1-(benzoyloxy)ethyl; isopropoxy-carbonyloxymethyl; 1-isopropoxy-carbonyloxyethyl; cyclohexyl-carbonyloxymethyl; 1-cyclohexyl-carbonyloxyethyl; cyclohexyloxy-carbonyloxymethyl; 1-cyclohexyloxy-carbonyloxyethyl; (4-tetrahydropyranyloxy) carbonyloxymethyl; 1-(4-tetrahydropyranyloxy)carbonyloxyethyl; (4-tetrahydropyranyl)carbonyloxymethyl; and 1-(4 tetrahydropyranyl)carbonyloxyethyl).

Also, some prodrugs are activated enzymatically to yield the active compound, or a compound which, upon further chemical reaction, yields the active compound. For example, the prodrug may be a sugar derivative or other glycoside conjugate, or may be an amino acid ester derivative.

### Complexes of the Compounds and Their Uses

The compounds of the present invention may be used for therapy, in particular the treatment of arthritis and cancer. In addition, the compounds of the present invention may be used to chelate radionuclides, for example to enable them to be employed in imaging studies or for therapeutic purposes. Examples of radionuclides that are chelatable by the compounds of the present invention include technetium, rhenium and copper isotopes such as ^{99m}Tc, ¹⁸⁶Re, ¹⁸⁸Re, ⁶⁷Cu, ⁶⁴Cu, ⁶²Cu, ⁶¹Cu, ⁶⁰Cu. The present invention may employ the radionuclides alone or in combinations. For example, one commonly used combination is ^{186/188}Re. Other combinations are ^{99m}Tc/¹⁸⁸Re or ^{99m}Tc/¹⁸⁶Re. In general, technetium isotopes are employed for imaging purposes, rhenium isotopes for therapeutic purposes and copper isotopes for both imaging and therapy. Where a specific isotope is not shown for an atom, it may be selected as any of the known isotopes or a mixture thereof.

The present invention provides active compounds for use in a method of treatment of the human or animal body. Such a method may comprise administering to such a subject a therapeutically-effective amount of an active compound, preferably in the form of a pharmaceutical composition.

The term “treatment”, as used herein in the context of treating a condition, pertains generally to treatment and therapy, whether of a human or an animal (e.g. in veterinary applications), in which some desired therapeutic effect is achieved, for example, the inhibition of the progress of the condition, and includes a reduction in the rate of progress, a halt in the rate of progress, amelioration of the condition, relief of pain, and cure of the condition. Treatment as a preventative measure, i.e. prophylaxis, is also included. By way of example, the compounds and complexes of the present invention may be used for the treatment of arthritis and for the treatment of cancer. The treatment of cancer may involve palliative and/or therapeutic treatment.

The term “therapeutically-effective amount” as used herein, includes that amount of an active compound, or a material, composition or dosage form comprising an active compound, which is effective for producing some desired therapeutic effect, commensurate with a reasonable benefit/risk ratio.

### Formulations and Dosage

While it is possible for the active compound to be administered alone, it is preferable to present it as a pharmaceutical composition (e.g. formulation) comprising at least one active compound, as defined above, together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents.

Thus, the present invention further provides pharmaceutical compositions, as defined above, and methods of making a pharmaceutical composition comprising admixing at least one active compound, as defined above, together with one or more pharmaceutically acceptable carriers, excipients, buffers, adjuvants, stabilisers, or other materials, as described herein.

The term “pharmaceutically acceptable” as used herein includes compounds, materials, compositions, and/or dosage forms which are, within the scope of sound medical judgement, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation. Suitable carriers, excipients, etc. can be found in standard pharmaceutical texts, for example, ‘Remington's Pharmaceutical Sciences’, 18th edition, Mack Publishing Company, Easton, Pa., 1990.

For intravenous, cutaneous or subcutaneous injection, or injection at the site of affliction, the active ingredient will be in the form of a parenterally acceptable aqueous solution or suspension which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets.

It will be appreciated that appropriate dosages of the active compounds, and compositions comprising the active compounds, can vary from patient to patient. Determining the optimal dosage will generally involve the balancing of the level of therapeutic benefit against any risk or deleterious side effects of the treatments of the present invention. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects.

Administration in vivo can be effected in one dose, continuously or intermittently (e.g. in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

### EXAMPLES

### Materials and Methods

All chemicals were supplied by Sigma-Aldrich or Fisher Scientific if not otherwise specified. Sodium (pertechnetate) (Na[^{99m}TcO₄]) in saline was supplied by Guy's and St Thomas' Hospital Nuclear Medicine Services. Cyclic RGD peptide (Arg-Gly-Asp-D-Phe-Lys, cyclised via the peptide backbone) and PSMAt peptide were purchased from Peptide Synthetics (Hampshire, UK).

NMR data (¹H, ¹³C{H}and ³¹P{H}1D spectra and COSY, TOCSY and HSQC spectra) were acquired on a Bruker Avance III 400 spectrometer equipped with a QNP probe or a Bruker Avance III 700 spectrometer equipped with an AVIII console and a quadruple-resonance QCI cryoprobe. High resolution mass spectrometry (MS) was performed by the King's College London Mass Spectrometry Facilities, using a high resolution Thermo Exactive mass spectrometer in positive electrospray mode. Samples were infused to the ion source at a rate of 10 l/min using a syringe pump. High performance liquid chromatography (HPLC) was carried out on an Agilent 1200 LC system with the Laura software, a Rheodyne sample loop (200 μL) and UV spectroscopic detection at 220 nm or 254 nm. The HPLC was attached to a LabLogic Flow-Count detector with a sodium iodide probe (B-FC-3200) for radiation detection. Semi-preparative (9.4×250 mm, 5 μm) and analytical (4.6×150 mm, 5 μm) Agilent Zorbax Eclipse XDB-C18 columns were used with purified water (A) and acetonitrile (B) containing 0.005% and 0.1% TFA as mobile phases for semi-preparative and analytical runs, respectively.

General HPLC methods used herein include; HPLC Method 1 (semi-preparative): 100 minutes, 1% min-1 linear increase from 100% A to 100% B, flow rate=3 ml min⁻¹. HPLC Method 2 (analytical): 20 minutes, 5% min⁻¹ linear increase from 100% A to 100% B (flow rate of 1 ml/min). HPLC Method 3 (semi-preparative): 200 minutes, 0.5% min⁻¹ linear increase from 95% A to 100% B (flow rate of 3 ml/min). HPLC Method 4 (analytical): 55 minutes, 2.5% min⁻¹ linear increase from 100% A to 25% B over 10 min, followed by 0.33% min⁻¹ linear increase from 25% A to 40% B over 45 min (flow rate of 1 mL min⁻¹).

Instant thin layer chromatography (iTLC) used iTLC SGIO001 strips (Varian Medical Systems, Crawley, UK). The iTLC plates were scanned with a Perkin Elmer Storage Phosphor System (Cyclone) or a LabLogic miniScan TLC reader equipped with Laura software.

High performance liquid chromatography (HPLC) was carried out on an Agilent 1200 HPLC system with Laura software, a Rheodyne sample loop (200 μL) and ultraviolet (UV) spectroscopic detection at 214 nm, 220 nm, 254 nm or 280 nm.

### Example 1—Synthesis of Compound (I-1); 3,4-bis(bisphenylphosphanyl)furan-2,5-dione (Compound (I-1)

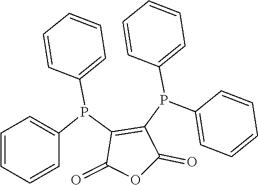 [SMILES: O=C1OC(=O)C(P(c2ccccc2)c2ccccc2)=C1P(c1ccccc1)c1ccccc1]

Diphenylphosphine (2.2 equiv., 5.04 mmol, 0.88 mL) was added to a solution of dichloromaleic anhydride (1 equiv., 2.42 mmol, 404.0 mg) in diethyl ether (15 mL) to give a pale-yellow solution. Triethylamine (2.2 equiv. 5.04 mmol, 0.7 mL) was added dropwise and the dark yellow suspension stirred (rt, 2 h) until a compact sludge had formed. The solids, which contained product, were isolated by filter cannula and washed with ice cold diethyl ether (3×10 mL). The crude product was re-dissolved and passed through a silica plug in dichloromethane, after which the solvent was removed under reduced pressure to yield a yellow solid. This product was recrystallised from chloroform/diethyl ether, furnishing crystalline yellow needles (390.7 mg, 837.7 mol, 34.6%).

¹H NMR (399 MHz, acetonitrile-d₃, 298 K): δ (ppm) 7.38-7.42 (m, 12H, Hₘₑₜₐ and Hₚₐᵣₐ), 7.34-7.30 (m, 8H, Hₒᵣₜₕₒ);

¹³C NMR (100 MHz, acetonitrile-d₃, 298 K): δ (ppm) 163.22 (m, C_{carbonyl}), 153.50 (m, Cₐₗₖₑₙₑ), 134.12 (m, Cₒᵣₜₕₒ), 133.00 (m, C_{subset}), 129.84 (m, Cₚₐᵣₐ), 128.73 (m, Cₘₑₜₐ);

³¹P{¹H}NMR (162 MHz, acetonitrile-d₃, 298 K): δ (ppm) −18.37;

³¹P{¹H}NMR (162 MHz, dimethylformamide-d₇, 298 K): δ (ppm) −19.07;

³¹P{¹H}NMR (162 MHz, chloroform-d₃, 298 K): δ (ppm) −20.53;

HR-MS-ESI m/z: [M+H]⁺ 467.0954 (Calculated for C₂₈H₂₁O₃P₂ 467.0960);

IR (solid) λₘₐₓ (cm⁻¹) 3054 (w), 1834 (m), 1811 (m), 1757 (s), 1496 (w), 1484(w), 1435 (m), 1244 (s), 913 (s);

m.p. 149.6° C.

### Example 2—Synthesis of Compound (I-2); 3,4-bis(bis-p-tolylphosphanyl)furan-2,5-dione (Compound (I-2))

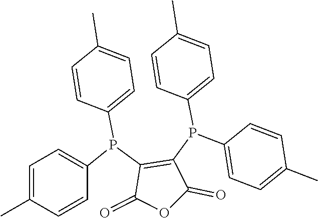

STEP 1: Bis(p-tolyl)chlorophosphine (1 equiv., 4.02 mmol, 0.9 mL) in diethyl ether (5 mL) was added dropwise to a slurry of lithium aluminium hydride (3.2 equiv., 13.01 mmol, 493.8 mg) in diethyl ether (20 mL) at 0° C. The grey suspension was stirred at 0° C. (30 min) and then at room temperature until reaction completion (22 h), determined by in situ ³¹P{¹H}NMR. The reaction was quenched by dropwise addition of i) degassed water (0.5 mL), ii) 15% NaOH_{(aq)} (0.5 mL) and iii) degassed water (1.5 mL) at 0° C.

The white precipitate was removed from the filtrate (that contained the product) by filter cannula. The precipitate was then washed with diethyl ether (2×10 mL) and these washes were combined with the filtrate. The resulting solution was dried on magnesium sulfate and re-isolated by filter cannula, washing the magnesium sulfate with diethyl ether (2×10 mL) and combining the filtrate and washes. The solvent was removed under reduced pressure to yield the product as a clear liquid (593.4 mg, 2.77 mmol, 68.9%) that crystallized below 20° C. When the reaction scale was doubled, the crude product was purified by distillation at 200° C. and 2.5×10-1 mbar.

1H NMR (400 MHz, Chloroform-d) δ 7.48-7.36 (m, 4H, Hb), 7.22-7.12 (m, 4H, He), 5.25 (d, JH-P=174.9 Hz, 1H, PH), 2.38 (s, 6H, He);

³¹P{¹H}NMR (162 MHz, Chloroform-d) δ −41.93;

³¹P NMR (162 MHz, Chloroform-d) δP −41.92 (d, J=174.9 Hz).

STEP 2: 3,4-bis(bis-o-tolylphosphanyl)furan-2,5-dione was prepared from (Tol)₂PH by the following method: A solution of ditolylphosphine (1.9 equiv., 0.36 mmol, 77.0 mg) in diethylether (0.2 mL) was added dropwise to a solution of dichloromaleic anhydride (1 equiv., 0.19 mmol, 31.0 mg) in tetrahydrofuran (1.3 mL) to give a clear orange solution. Triethylamine (3 equiv. 0.58 mmol, 0.08 mL) was added dropwise and the dark orange suspension stirred (rt, 2 h). The solids were removed by filter cannula, washing with tetrahydrofuran (3×2 mL). The filtrate and washes (that contained the product) were combined and the solvent removed from the resulting product solution under reduced pressure. The crude product was re-dissolved and passed through a silica plug in dichloromethane and the solvent removed under reduced pressure. The product was dissolved in a minimal amount of chloroform and the solution layered with diethyl ether. The precipitate was collected by filtration and dried to yield the product as yellow needle crystals (30.2 mg, 0.06 mmol, 16.1%).

¹H NMR (500 MHz, Chloroform-d) δH 7.21 (dt, J=8.6, 4.4 Hz, 8H, Hₒᵣₜₕₒ), 7.08 (d, J=7.7 Hz, 8H, Hₘₑₜₐ), 2.34 (s, 12H, H_{para-Methyl});

¹³C{¹H}NMR (125 MHz, Chloroform-d): δC (ppm) 162.84 (t, J=2.86, C_{carbonyl}), 155.03 (m, Cₐₗₖₑₙₑ), 140.06 (s, Cₚₐᵣₐ), 134.24 (m, Cₒᵣₜₕₒ), 129.60 (t, J=4.40, Cₘₑₜₐ), 129.04 (m, C_{subst}), 21.55 (s, C_{para-Methyl});

³¹P{¹H}NMR (162 MHz, Chloroform-d) δP −23.08 (s);

HR-MS-ESI m/z: [M+H]⁺ 523.1602 (calculated for C₃₂H₂₈O₃P₂ 523.1592).

### Example 3a—Synthesis of bis(para-methoxyphenyl )phosphine((p-MeOC₆H₄)₂PH)

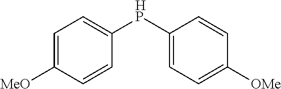

A solution of bis(4-methoxyphenyl)chlorophosphine (1 g, 3.56 mmol) in Et₂O (4.5 mL) was added dropwise to a suspension of LiAlH₄ (1.24 g, 11.4 mmol, 3.2 eq) in Et₂O (18 mL) at 0° C. The solution was stirred for a further 30 min at 0° C. before allowing to warm to RT and then stirred overnight. The reaction mixture was cooled to 0° C. and quenched by the careful addition of H₂O (0.5 mL), 15% NaOH (0.5 mL) and H₂O (2.5 mL). After stirring for 1 hr, the solution was isolated by filtration and then concentrated in vacuo to give the title compound (744 mg, 3.02 mmol, 85%) as a white solid.

¹H NMR (400 MHz, CDCl₃): δ_{H} (ppm) 7.46-7.28 (m, 4H, Ar—H), 6.90-6.82 (m, 4H, Ar—H), 5.38-4.98 (br. s, PH), 3.80 (s, 6H, OMe).

³¹P{¹H}NMR (162 MHz, CDCl₃): δ_{P} (ppm) −44.2 (s). The spectroscopic data is in accordance with the literature (Y. Y. Yan and T. V. RajanBabu, Org. Lett., 2000, 2, 4137-4140).

### Example 3b—Synthesis of Compound (I-11); 3,4-bis[bis(4-methoxyphenyl)phosphanyl]furan-2,5-dione

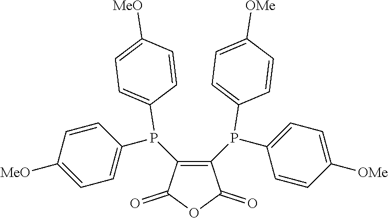 [SMILES: COc1ccc(P(C2=C(P(c3ccc(OC)cc3)c3ccc(OC)cc3)C(=O)OC2=O)c2ccc(OC)cc2)cc1]

NEt₃ (30.4 μL, 2.18 mmol, 2.2 eq) was added to a solution of (p-MeOC₆H₄)₂PH (50.0 mg, 0.203 mmol, 2.05 eq) in Et₂O (0.5 mL). A solution of 2,3-dichloromaleic anhydride (16.5 mg, 98.8 μmol) in Et₂O (0.5 mL) was added dropwise, which resulted in an immediate colour change from colourless to deep red solution. Once the reaction had reached completion, as monitored by ³¹P NMR spectroscopy, the volatiles were removed in vacuo. The crude product was dissolved in DCM and passed through a silica plug (2% MeOH in DCM) and concentrated to dryness. Residual (p-MeOC₆H₄)₂PH was removed under high vacuum (c.a. ×10⁻⁷ Torr) to give the title compound (51.4 mg, 87.7 μmol, 89%) as an orange solid.

³¹P{¹H}NMR (162 MHz, CDCl₃): δ_{P} (ppm) −22.3 (s).

¹H NMR (400 MHz, CDCl₃): δ_{H} (ppm) 7.28-7.21 (m, 8H, Ar—H), 6.83-6.78 (m, 8H, Ar—H), 3.80 (s, 12H, OMe).

¹³C NMR (101 MHz, CDCl₃): δ_{C} (ppm) 163.0 (m, C═O), 161.1 (s, p-ArC), 154.2 (m, C═C), 135.9 (t, ²J_{P,C}=12.2, o-ArCH), 123.4 (s, ArC), 114.5 (t, ³J_{P,C}=4.9 Hz, m-ArCH), 55.3 (s, OMe).

HR-MS (Nanospray): m/z calcd. for C₃₂H₂₉O₇P₂ [M+H]⁺=587.1389; obs.=587.1395.

### Example 4—Synthesis of PEG-PSMA Peptide Conjugates (II-1-PSMAt1), (II-2-PSMAt1) and (II-11-PSMAt1)

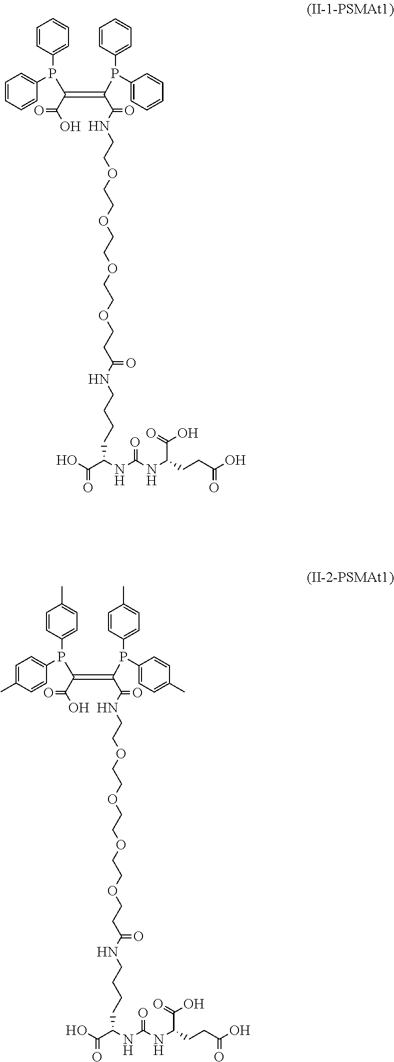 [SMILES: O=C(O)CC[C@H](NC(=O)N[C@@H](CCCCNC(=O)CCOCCOCCOCCOCCNC(=O)/C(=C(\C(=O)O)P(c1ccccc1)c1ccccc1)P(c1ccccc1)c1ccccc1)C(=O)O)C(=O)O ; Cc1ccc(P(/C(C(=O)O)=C(/C(=O)NCCOCCOCCOCCOCCC(=O)NCCCC[C@H](NC(=O)N[C@@H](CCC(=O)O)C(=O)O)C(=O)O)P(c2ccc(C)cc2)c2ccc(C)cc2)c2ccc(C)cc2)cc1]

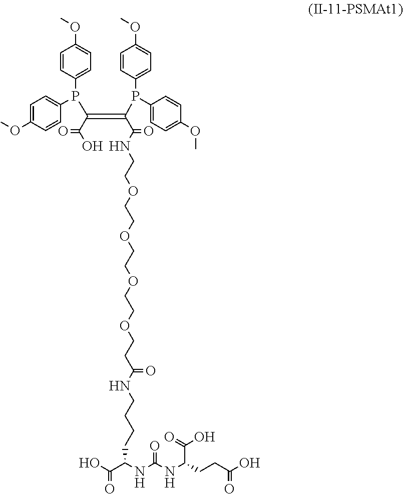 [SMILES: COc1ccc(P(/C(C(=O)O)=C(/C(=O)NCCOCCOCCOCCOCCC(=O)NCCCC[C@H](NC(=O)N[C@@H](CCC(=O)O)C(=O)O)C(=O)O)P(c2ccc(OC)cc2)c2ccc(OC)cc2)c2ccc(OC)cc2)cc1]

Under a stream of nitrogen, Compound (I-1), Compound (I-2) or Compound (I-11) (5-10 mg, 1 equiv.) in DMF (100 μL, dry, degassed) and Lys-((PEG)₄—NH₂)-uredo-Glu, 5-10 mg, 1 equiv.) in DMF (100 μL, dry, degassed) were combined and N,N-diisopropylethylamine (DIPEA, 6 μL) added. The tube was sealed and the solution agitated at room temperature (15-20 min). The product was isolated by semi-preparative C₁₈-HPLC (mobile phases: 0.01% acetic acid in water (A) and acetonitrile (B); method starting at 95% A and increasing to 100% B; unreacted compound elutes at 100% acetonitrile). Product-containing fractions were neutralised with aqueous ammonium bicarbonate buffer (0.125 M, 15 μL/mL elute) and freeze-dried to yield the PSMAt1 peptide conjugate (>60.0%) as a solid.

The reaction is reversible under acidic conditions, but simple addition of ammonium bicarbonate to solutions of isolated material prevents this.

### Characterization of Compound (II-1-PSMAt1)

(700 MHz, DMF-d₇, 298 K): δ (ppm) 1.382-1.436 (m, 2H, Lys, H_{γ}), 1.445-1.504 (m, 2H, Lys, H_{δ}), 1.608-1.660 (m, 1H, Lys, H_{β}), 1.742-1.795 (m, 1H, Lys, H_{β}), 1.842-1.893 (m, 1H, Glu, H_{β}), 1.986-2.039 (m, 1H, Glu, H_{β}), 2.324-2.364 (m, 1H, Glu, H_{γ}), 2.386 (t, J=6.24 Hz, 2H, PEG, Hₒ), 2.455 (dt, J₁=14.68 Hz, J₂=8.39 Hz, 1H, Glu, H_{γ}), 2.955-2.983 (m, 2H, PEG, Hₕ), 3.014-3.045 (m, 2H, PEG, Hᵢ), 3.128 (dd, J₁=12.81 Hz, J₂=6.46 Hz, 2H, Lys, H_{ε}), 3.417-3.431 (m, 2H, PEG, Hⱼ₋ₒ), 3.520-3.591 (m, 10H, PEG, Hⱼ₋ₒ), 3.683 (t, J=6.24 Hz, 2H, PEG, Hₚ), 4.204-4.233 (m, 1H, Lys, H_{α}), 4.261-4.285 (m, 1H, Glu, H_{α}), 6.543 (m, 1H, Glu, NH), 6.639 (d, J=7.48 Hz, 1H, Lys, NH), 7.208-7.257 (m, 12H, DP^{Ph}, H_{e/f}), 7.431-7.466 (m, 4H, DP^{Ph}, CH_{d/d′}), 7.574-7.601 (m, 4H, DP^{Ph}, H_{d/d′}), 7.806 (t, J=5.44 Hz, 1H, PEG, NH), 7.828 (t, J=5.67 Hz, 1H, Lys, NH_{ζ}); ¹³C NMR (176 MHz, DMF-d₇, 298 K): δ (ppm), 23.063 (s, Lys, C_{γ}), 29.320 (Lys, C_{δ}), 29.840 (Glu, C_{β}), 32.208 (s, Glu, C_{γ}), 32.673 (s, Lys, C_{β}), 36.718 (s, PEG, C_{q}), 38.739 (s, PEG, Cₕ), 38.834 (s, Lys, C_{ε}), 53.364 (s, Glu, C_{α}), 53.498 (s, Lys, C_{α}), 67.398 (s, PEG, Cₚ), 69.051 (s, PEG, Cᵢ), 70.096 (s, PEG, Cⱼ₋ₒ), 70.217 (s, PEG, Cⱼ₋ₒ), 70.292 (s, PEG, Cⱼ₋ₒ), 70.419 (s, PEG, Cⱼ₋ₒ), 70.430 (s, PEG, Cⱼ₋ₒ), 127.814 (d, J=6.78 Hz, DP^{Ph}, C_{e/e′}), 127.870 (d, J=7.35 Hz, DP^{Ph}, C_{e/e′}), 128.150 (s, DP^{Ph}, C_{f/f′}), 128.379 (s, DP^{Ph}, C_{f/f′}), 134.035 (dd, J₁=19.47 Hz, J₂=5.82 Hz, DP^{Ph}, C_{d/d′}), 134.653 (d, J=20.35 Hz, DP^{Ph}, C_{d/d′}), 136.936 (m, DP^{Ph}, C_{c/c′}), 137.650 (m, DP^{Ph}, C_{c/c′}), quaternary carbons: 157.065 (s), 170.452 (s), 174.729 (s), 175.006 (s), 175.232 (s), remaining signals corresponding to quaternary carbons could not be distinguished from noise; ³¹P{¹H}NMR (283 MHz, DMF-d₇, 298 K): δ (ppm) −13.18 (d, J=162.7 Hz), −12.15 (d, J=162.7 Hz).

HR-MS-ESI m/z: [M+H]⁺ 1033.3759 (calculated for C₅₁H₆₃O₁₅N₄P₂ 1033.3760), [M+Na]⁺ 1055.3579 (calculated for C₅₁H₆₂O₁₅N₄P₂Na 1055.3579).

### Characterization of Compound (II-2-PSMAt1)

¹H NMR (700 MHz, DMF-d₇, 298 K): δ (ppm) 1.389-1.444 (m, 2H, Lys, H_{γ}), 1.453-1.503 (m, 2H, Lys, H_{δ}), 1.618-1.670 (m, 1H, Lys, H_{β}), 1.752-1.801 (m, 1H, Lys, H_{β}), 1.899-1.949 (m, 1H, Glu, H_{β}), 1.983-2.035 (m, 1H, Glu, H_{β}), 2.262 (s, 6H, DP^{Tol}, H_{g/g′}), 2.293 (s, 6H, DP^{Tol}, H_{g/g′}), 2.345-2.384 (m, 1H, Glu, H_{γ}), 2.389 (t, J=6.25 Hz, 2H, PEG, Hₒ), 2.451 (dt, J₁=15.00 Hz, J₂=8.17 Hz, 1H, Glu, H_{γ}), 2.962-3.008 (m, 4H, PEG, H_{h/i}), 3.139 (hidden, 2H, Lys, H_{ε}), 3.407-3.421 (m, 2H, PEG, Hⱼ₋ₒ), 3.524-3.591 (m, 10H, PEG, Hⱼ₋ₒ), 3.685 (t, J=6.25 Hz, 2H, PEG, Hₚ), 4.217-4.247 (m, 1H, Lys, H_{α}), 4.268-4.294 (m, 1H, Glu, H_{α}), 6.559 (d, J=4.84 Hz, 1H, Glu, NH), 6.631 (d, J=7.72 Hz, 1H, Lys, NH), 7.005 (d, J=7.63 Hz, 4H, DP^{Ph}, Hₑ), 7.036 (d, J=7.62 Hz, 4H, DP^{Ph}, Hₑ), 7.325 (dd, J₁=14.22 Hz, J₂=7.36 Hz, 4H, DP^{Ph}, CH_{d/d′}), 7.427 (t, J=7.68 Hz, 4H, DP^{Ph}, H_{d/d′}), 7.799-7.824 (m, 1H, PEG, NH), 7.799-7.824 (m, 1H, Lys, NH_{ζ}); ¹³C NMR (176 MHz, DMF-d₇, 298 K): δ (ppm) 20.667 (s, DP^{Tol}, C_{g/g′}), 20.686 (s, DP^{Tol}, C_{g/g′}), 23.064 (s, Lys, C_{γ}), 29.306 (hidden, Lys, C_{δ}), 29.680 (hidden, Glu, C_{β}), 31.929 (s, Glu, C_{γ}), 32.656 (s, Lys, C_{β}), 36.726 (s, PEG, C_{q}), 38.703 (s, PEG, Cₕ), 38.840 (s, Lys, C_{ε}), 53.328 (s, Glu, C_{α}), 53.453 (s, Lys, C_{α}), 67.389 (s, PEG, Cₚ), 69.058 (s, PEG, Cᵢ), 70.176 (s, PEG, Cⱼ₋ₒ), 70.223 (s, PEG, Cⱼ₋ₒ), 70.308 (s, PEG, Cⱼ₋ₒ), 70.436 (s, PEG, Cⱼ₋ₒ), 70.454 (s, PEG, Cⱼ₋ₒ), 128.505 (d, J=7.07 Hz, DP^{Tol}, C_{e/e′}), 128.568 (d, J=7.35 Hz, DP^{Tol}, C_{e/e′}), 134.197 (d, J=19.96 Hz, DP^{Tol}, C_{d/d′}), 134.675 (d, J=20.96 Hz, DP^{Tol}, C_{d/d′}), 137.682 (s, DP^{Tol}, C_{c/c′}), 137.949 (s, DP^{Tol}, C_{f/f′}), quaternary carbons: 158.097 (s), 170.435 (s), 174.665 (s), 175.003 (s), 175.179 (s), remaining signals corresponding to quaternary carbons could not be distinguished from noise; ³¹P{¹H}NMR (283 MHz, DMF-d₇, 298 K): δ (ppm) −15.776 (d, J=151.40 Hz), −14.392 (d, J=151.40 Hz).

HR-MS-ESI m/z: [M+H]⁺ 1089.4373 (calculated for C₅₅H₇₁O₁₅N₄P₂ 1089.4386), [M+Na]⁺ 1111.4193 (calculated for C₅₅H₇₀O₁₅N₄P₂Na 1111.4205), [M+MeOH+H]⁺ 1121.4275 (calculated for C₅₆H₇₅O₁₆N₄P₂ 1121.4648).

### Characterization of Compound (II-11-PSMAt1)

³¹P NMR 283 MHz, DMF-d₇, 298 K): δ (ppm) −17.21 (d, J=139.3 Hz), −19.24 (d, −139.3 Hz).

HRMS: [M+H]⁺ 1153.4196 (observed), 1153.4182 (calculated)

### Example 5—Preparation and Characterisation of the Cis/Trans Re-Complex of the Conjugated Peptide RGD Using Compound (^{nat}Re-III-1-RGD)/Compound (II-1-RGD)

The chemistry of Re and Tc is similar. As Tc has no stable isotopes, it was convenient to prepare Compound (^{nat}Re-III-1-RGD)/[^{nat}ReO₂(II-1-RGD)₂]⁺ to obtain full characterisation.

A solution of [^{nat}ReO₂I(PPh₃)₂](3.0 mg, 3.45 μmol) in DMF (100 μL) was combined with a solution of Compound (II-1-RGD) (3.7 mg, 3.45 mol) and DIPEA (6 μL) in DMF (200 μL). The resulting dark brown/black solution was agitated at room temperature for 10 min. Upon addition of ice-cold diethyl ether, a precipitate formed. The supernatant was removed, and the precipitate was dissolved in DMF (200 μL) and applied to a semi-preparative HPLC column. Reaction components were separated using HPLC method 3. A solution of aqueous ammonium bicarbonate (0.125 M) was added to each fraction containing cis/trans-[^{nat}ReO₂(II-1-RGD)₂]⁺ at a ratio of 10 μL of ammonium acetate solution: 1 mL of HPLC eluate. Solutions containing cis/trans-[^{nat}ReO₂(II-1-RGD)₂]⁺ were lyophilised. The lyophilised fractions that eluted at 65-67 min and 68-70 min were identified as trans-[^{nat}ReO₂(II-1-RGD)₂]⁺ (0.8 mg, 0.34 μmol, 9.9%) and cis-[^{nat}ReO₂(II-1-RGD)₂]⁺ (0.9 mg, 0.38 μmol, 11.0%), respectively.

### Characterising Data for trans-[^{nat}ReO₂(II-1-RGD)₂]⁺

¹H NMR (700 MHz, DMF-d₇, 298 K): δ (ppm) 1.184 (m, 4H, Lys, γ CH₂), 1.300 (m, 4H, Lys, δ CH₂), 1.511 (m, 2H, Arg, β CH), 1.602 (m, 2H, Lys, β CH), 1.612 (m, 2H, Arg, β CH), 1.681 (m, 2H, Lys, β CH), 1.769 (m, 4H, Arg, γ CH₂), 2.261 (m, hidden, Asp, β CH), 2.601 (dd, J1=14.51 Hz, J₂=9.26 Hz, 2H, Phe, β CH), 2.940 (m, hidden, Asp, β CH), 2.943 (hidden, Lys, ε CH₂), 3.067 (m, 2H, Arg, δ CH), 3.144 (m, 2H, Arg, δ CH), 3.33 (dd, J1=9.26 Hz, J₂=5.00 Hz, 2H, Phe, β CH), 3.416 (dd, J₁=16.45 Hz, J₂=9.05 Hz, 2H, Gly, α CH), 4.307 (dd, J1=16.24 Hz, J₂=2.67 Hz, 2H, Gly, α CH), 4.358 (m, 2H, Asp, α CH), 4.365 (m, 2H, Lys, α CH), 4.543 (m, 2H, Arg, α CH), 4.796(m, 2H, Phe, α CH), 7.111 (m, PPh₂, aromatic CHₘ), 7.161 (m, Phe, aromatic CHₚ), 7.170 (m, PPh₂, aromatic CHₘ), 7.204 (m, PPh₂, aromatic CHₒ), 7.232 (m, Phe, aromatic CHₒ and CHₘ), 7.281 (m, PPh₂, aromatic CHₒ), 7.436 (m, PPh₂, aromatic CHₚ), 7.477 (m, PPh₂, aromatic CHₚ), 7.787 (d, J=8.98 Hz, 2H, Phe, NH), 8.089 (m, 2H, Gly, NH), 8.196 (m, 2H, Lys, NH), 8.294 (m, 2H, Lys, ε NH), 8.358 (d, J=9.32 Hz, 2H, Asp, NH), 8.629 (d, J=8.98 Hz, 2H, Arg, NH).

¹³C NMR (176 MHz, CD₃CN, 298 K): δ (ppm) 15.56 (s, Lys, γ CH₂), 24.66 (s, Arg, β CH₂), 27.14 (s, Arg, γ CH₂), 29.47 (s, Lys, δ CH₂), 32.87 (s, Lys, β CH₂), 36.86 (s, Phe, β CH₂), 38.77 (s, Asp, β CH₂), 38.98 (s, Lys, ε CH₂), 41.04 (s, Arg, δ CH₂), 43.11 (s, Gly, α CH₂), 49.12 (s, Asp, α CH), 51.32 (s, Arg, α CH), 53.36 (s, Phe, α CH), 55.59 (s, Lys, α CH), 118.09 (m, PPh₂, aromatic Cₛ), 125.99 (s, Phe, aromatic Cₚ), 127.55 (m, PPh₂, aromatic Cₘ), 128.02 (s, Phe, aromatic Cₒ or Cₘ), 129.31 (s, Phe, aromatic Cₒ or Cₘ), 131.130 (m, PPh₂, aromatic Cₚ), 134.63 (m, PPh₂, aromatic Cₒ), 158.21-172.63 (>9 signals for X=CR₂; where X is N, O or C).

³¹P NMR (283 MHz, DMF-d₇, 298 K): δ (ppm) 23.781 (m), 24.506 (m).

HR-MS-ESI m/z: [M+H]²⁺ 1179.3826 (Calculated for C₁₁₀H₁₂₂N₁₈O₂₂P₄Re⁺ 1179.3773), [M+2H]³⁺ 786.5921 (Calculated for C₁₁₀H₁₂₂N₁₈O₂₂P₄Re⁺ 786.5906).

### Characterising Data for Cis-[^{nat}ReO₂(II-1-RGD)₂]⁺

¹H NMR (700 MHz, DMF-d₇, 298 K): δ (ppm) 1.188 (m, 4H, Lys, γ CH₂), 1.300 (m, 4H, Lys, δ CH₂), 1.528 (m, 2H, Arg, β CH), 1.599 (m, 2H, Lys, β CH), 1.608 (m, 2H, Arg, β CH), 1.664 (m, 2H, Lys, β CH), 1.785 (m, 4H, Arg, γCH₂), 2.283 (m, 2H, Asp, β CH), 2.606 (m, 2H, Phe, β CH), 2.876 (m, 4H, Lys, ε CH₂), 2.924 (hidden, Asp, β CH), 3.109 (m, 2H, Arg, δ CH), 3.148 (m, 2H, Arg, δ CH), 3.319 (dd, J₁=14.51 Hz, J₂=5.14 Hz, 2H, Phe, β CH), 3.415 (hidden, Gly, α CH), 4.308 (dd, J₁=16.64 Hz, J₂ =9.05 Hz, 2H, Gly, α CH), 4.367 (m, 2H, Lys, α CH), 4.379 (m, 2H, Asp, α CH), 4.525 (m, 2H, Arg, α CH), 4.789 (m, 2H, Phe, α CH), 7.151 (m, PPh₂, aromatic CHₘ), 7.158 (m, Phe, aromatic CHₚ), 7.175 (m, PPh₂, aromatic CHₒ), 7.220 (m, Phe, aromatic CHₒ and CHₘ), 7.305 (m, PPh₂, aromatic CHₒ), 7.439 (m, PPh₂, aromatic CHₚ), 7.798 (d, J=9.37 Hz, 2H, Phe, NH), 8.087 (m, 2H, Gly, NH), 8.088 (m, 2H, Lys, ε NH), 8.253 (d, J=7.46 Hz, 2H, Lys, NH), 8.335 (d, J=8.85 Hz, 2H, Asp, NH), 8.621 (d, J=8.85 Hz, 2H, Arg, NH).

¹³C NMR (176 MHz, CD₃CN, 298 K): δ (ppm) 15.56 (s, Lys, γ CH₂), 24.32 (s, Arg, β CH₂), 27.09 (s, Arg, γ CH₂), 29.54 (hidden, Lys, δ CH₂), 32.84 (s, Lys, β CH₂), 36.81 (s, Phe, β CH₂), 38.63 (s, Asp, β CH₂), 38.91 (s, Lys, ε CH₂), 41.04 (s, Arg, δ CH₂), 43.09 (s, Gly, α CH₂), 49.21 (s, Asp, α CH), 51.37 (s, Arg, α CH), 53.37 (s, Phe, α CH), 55.52 (s, Lys, α CH), 118.05 (m, PPh₂, aromatic Cₛ), 125.99 (s, Phe, aromatic Cₚ), 127.55 (m, PPh₂, aromatic Cₘ), 128.02 (s, Phe, aromatic Cₒ or Cₘ), 129.31 (s, Phe, aromatic Cₒ or Cₘ), 131.04 (m, PPh₂, aromatic Cₚ), 134.27 (m, PPh₂, aromatic Cₒ), 143.80 (m, PPh₂, aromatic Cₒ), 158.03-185.17 (several signals for X=CR₂; where X is N, O or C; too weak to characterise).

³¹P NMR (283 MHz, DMF-d₇, 298 K): δ (ppm) 21.848 (dm, J₁=356.1 Hz), 26.335 (dm, J₁=356.1 Hz).

HRMS-ESI m/z: [M+H]²⁺ 1179.3826 (Calculated for C₁₁₀H₁₂₂N₁₈O₂₂P₄Re⁺ 1179.3773), [M+2H]³⁺ 786.5921 (Calculated for C₁₁₀H₁₂₂N₁₈O₂₂P₄Re⁺ 786.5906).

### Example 6—Preparation and Characterisation of Compound (Tc-III-1-RGD)/[^{99m}TcO₂(II-1-RGD)₂]+

### Preparation of Radiolabelling Kits

An aqueous stock solution was prepared containing the required amounts of sodium bicarbonate, tin(II) chloride dihydrate, sodium gluconate or sodium tartrate dibasic dihydrate. The pH of this solution was adjusted to 8.5 by dropwise addition of an aqueous solution of sodium hydroxide (0.1M). Aliquots of the stock solution were mixed with the required amount of Compound (II-1-RGD) (in ethanol), and the resulting solutions were frozen and lyophilised. The lyophilised kits were stored at −18° C. prior to use.

### ^{99m}Tc Radiolabelling

Compound (II-1-RGD) was radiolabelled with generator-produced ^{99m}TcO₄⁻ in saline solution (0.9% NaCl in water, w/v). For each radiolabelling, a radiolabelling kit was thawed and reconstituted with a total of 300 μL of saline, ^{99m}TcO₄⁻ in saline solution and ethanol. The reconstituted kit was heated at 60° C. for 30 min, and then analysed by analytical HPLC (method 2) and instant thin layer chromatography (iTLC) using iTLC SGI0001 strips (9 or 10 cm length; Varian Medical Systems, Crawley, UK). The iTLC plates were scanned with a Perkin Elmer Storage Phosphor System (Cyclone) or a LabLogic miniScan TLC reader equipped with Laura software.

Two separate iTLC analyses were undertaken, to enable quantification of ^{99m}Tc-colloids, unreacted ^{99m}TcO₄⁻ and Compound (Tc-III-1-RGD).

To quantify amounts of unreacted ^{99m}TcO₄⁻, acetone was used as a mobile phase: R_{f} values: ^{99m}TcO₄⁻>0.9, ^{99m}Tc colloids<0.1, Compound (Tc-III-1-RGD)<0.1.

To quantify ^{99m}Tc-colloid formation, a 1:1 mixture of methanol and 2M aqueous ammonium acetate solution was used as a mobile phase: ^{99m}TcO₄⁻>0.9, ^{99m}Tc colloids<0.1, Compound (^{99m}Tc-III-1-RGD)>0.9.

Co-elution of (Tc-III-1-RGD) with cis/trans-(^{nat}Re-III-1-RGD): [^{99m}TcO₂(II-1-RGD)₂]⁺ was prepared in >90% RCY as described above, and co-injected with cis-[^{nat}ReO₂(II-1-RGD)₁]⁺ and separately, trans-[^{nat}ReO₂(II-1-RGD)₂]⁺, onto a reverse-phase analytical HPLC column (method 4). Retention times: trans/cis-(Tc-III-1-RGD) 41.0 min and 44.1 min (NaI scintillator detection); trans-(^{nat}Re-III-1-RGD) 38.3 min and cis-(^{nat}Re-III-1-RGD) 42.6 min.

### Log D (pH 7.4)

The following procedure was carried out in triplicate. A solution containing (^{99m}Tc-III-1-RGD) (1 MBq in 7.5 μL) was combined with phosphate buffered saline (pH 7.4, 500 μL) and octanol (500 μL), and the mixture was agitated for 30 min. The mixture was then centrifuged (10 000 rpm, 10 minutes), and aliquots of octanol and aqueous PBS were analysed for radioactive using a gamma counter. log D_{OCT/PBS}=−1.64±0.04.

### Serum Stability:

A solution containing Compound (Tc-III-1-RGD) (100 μL, 79 MBq) was added to filtered human serum (Sigma-Aldrich, 900 μL) and incubated at 37° C. for 4 h. At 1 and 4 h, aliquots were taken. Each aliquot (300 μL) was treated with ice-cold acetonitrile (300 μL) to precipitate and remove serum proteins. Acetonitrile in the supernatant was then removed by evaporation under a stream of N₂ gas (40° C., 30 min). The final solution was then analysed by reverse-phase analytical HPLC (method 2).

αvβ3-Integrin Solid-Phase Competitive Binding Assay:

The affinity of Compound (Tc-III-1-RGD) for αvβ3 integrin was determined in a solid-phase competitive binding assay. In brief, wells of a 96 well plate were coated with 150 ng/mL integrin αvβ3 in 100 μL coating buffer (25 mM Tris HCl pH 7.4, 150 mM NaCl, 1 mM CaCl₂, 0.5 mM MgCl₂, and 1 mM MnCl₂) overnight at 4° C. Wells were then washed twice in binding buffer (coating buffer plus 0.1% bovine serum albumin (BSA)) before being blocked for 2 hours at room temperature with blocking buffer (coating buffer plus 1% BSA). After a further two washes in binding buffer, both (Tc-III-1-RGD) (RCY >96%, 1-2 kBq in 50 μL binding buffer, containing 1.2 μmol Compound (II-1-RGD) peptide) and RGD peptide (10.0 μM to 10,000 nM, 50 μL in binding buffer) were added simultaneously to wells, and left to incubate for 1 h at room temperature, before being washed twice as before. Finally, the amount of activity bound to the wells was counted.

Binding of Compound (Tc-III-1-RGD) to αvβ3 integrin was displaced by RGD peptide in a concentration-dependent manner. The pseudo-IC₅₀ value of 8.54±3.45 nM (95% CI: 1.67-15.41 nM) was calculated using a non-linear regression model (Binding/Saturation, one site—total) in GraphPad Prism (n=6 from one experiment).

### Example 7—Pre-clinical imaging and in vivo biodistribution studies of Compound (Tc-III-1-RGD)/[^{99m}TcO₂(II-1-RGD)₂]⁺

Animal imaging studies were ethically reviewed and carried out in accordance with the Animals (Scientific Procedures) Act 1986 (ASPA) UK Home Office regulations governing animal experimentation. SPECT/CT imaging was accomplished using a pre-clinical nanoScan SPECT/CT Silver Upgrade instrument (Mediso) calibrated for technetium-99m. All scans were acquired by helical SPECT (4-head scanner with 4×9 [1.4 mm] pinhole collimators), and helical CT with 1.4 mm aperture collimators. All acquired images were reconstructed using a full 3D Monte Carlo-based iterative algorithm (Tera-Tomo; Mediso) and further processed and analysed using VivoQuant software (inviCRO, USA).

### SPECT/CT Imaging and Biodistribution in Healthy Mice

A female, balb/c mouse (2 months old) was anaesthetised (2-3% v/v isofluorane in oxygen), scanned by CT and injected intravenously (tail vein) with Compound (Tc-III-1-RGD) (21 MBq containing 22 μg of Compound (II-1-RGD) peptide). SPECT images (8×30 min images) were acquired over 4 h. At the end of the imaging procedure, the mouse was culled by cervical dislocation and a sample of the urine analysed by reverse-phase HPLC (analytical, method 2).

Female balb/c mice (2 months old) were anaesthetised (2-3% v/v isofluorane in oxygen) and injected intravenously (tail vein) with (^{99m}Tc-III-1-RGD) (2.7-5.3 MBq containing 5 μg of Compound (II-1-RGD)). For blocking studies, animals were co-injected with RGD peptide (400 μg). Mice remained under anaesthetic for 1 h, after which they were culled (pentabarbitone by i.v. injection). Tissues and organs were harvested and weighed, and radioactivity counted using a Gamma Counter (Wallac 1282 CompuGamma Universal Gamma Counter).

SPECT/CT Imaging and Biodistribution in Mice Induced with Rheumatoid Arthritis

An AK/BxN serum transfer arthritis (STA) model of rheumatoid arthritis was used (P. A. Monach, et al, Curr. Protoc. Immunol., 2008, 81, 15.22.1-15.22.12 and C. Imberti et al, Bioconjugate Chem., 2017, 28, 481-495). On day 0 and 2, female C57Bl/6J mice (2 months old) were injected intraperitoneally with arthritogenic serum in sterile filtered PBS (150 μL, 50% v/v, serum obtained from arthritic K/BxN transgenic mice). Disease severity was evaluated in mice throughout the induction period, by measuring weight, thickness of swollen paws using microcallipers, and visual scoring on a scale of 0-3 per paw. SPECT/CT imaging and biodistribution was undertaken on day 7.

Mice were anesthetised (2.5-3% v/v isofluorane) and their paws were measured using microcallipers. Mice were then injected intravenously with Compound (Tc-III-1-RGD) (approx. 5 MBq containing 5 μg of Compound (II-1-RGD)) and allowed to recover from anaesthetic administration. At 1 h post-injection of radiotracer, mice were culled (sodium pentabarbitone), and underwent SPECT/CT scanning post-mortem for 60-180 min. Finally, tissues and organs were harvested and weighed, and radioactivity counted using a Gamma Counter (Wallac 1282 CompuGamma Universal Gamma Counter). The acquired images were processed to units of % ID and the regions of interest (ROIs) delineated by CT using VivoQuant software (inviCRO, USA). Radioactivity in ankle and wrist ROIs were obtained in units of % ID and % ID/cm⁻³. Each ankle ROI was defined as the area between the tibiofibula joint and the base of phalanx V. Each “wrist” ROI was defined as the area between the narrowest point of the wrist (ulna and radius) and the end of the forepaw.

### Example 8—Preparation and characterisation of Compound (Tc-III-1-PSMAt1)/[^{99m}TcO₂(II-1-PSMAt1)₂]⁺ Compound (Tc-III-2-PSMAt1)/^{99m}TcO₂(II-2-PSMAt1)₂]⁺ and Compound (Tc-III-11-PSMAt1)/(^{99m}TcO₂(II-11-PSMAt1)₂]⁺

Kit preparation: An aqueous stock solution was prepared containing the required amounts of sodium bicarbonate, tin chloride and sodium tartrate. The pH was adjusted to either 7.5 or 8-8.5 by dropwise addition of an aqueous solution of either sodium hydroxide (0.1 M) or hydrochloric acid (0.1 M). Aliquots of the stock solution were mixed with the required amount of (II-1-PSMAt1), (II-2-PSMAt1), or (II-11-PSMAt1) (dissolved in a mixture of water/ethanol (50%/50%)) to form the kit solutions outlined in the table below, which were immediately frozen and lyophilised using a freeze dryer. The lyophilised kits were stored in a freezer prior to use.

TABLE 5
Lyophilised kit formulations for (II-1-PSMAt1),
(II-2-PSMAt1) and (II-11-PSMAt1) for radiolabelling.

| | Kit Compositions | | | | | |
|---|---|---|---|---|---|---|
| | (II-1-PSMAt1) | | (II-2-PSMAt1) | | (II-11-PSMAt1) | |
| | Kit | | Kit | | Kit | |
| | moles/ | weight/ | moles/ | weight/ | moles/ | weight/ |
| Components: | μmol | mg | μmol | mg | μmol | mg |
| (II-1-PSMAt1) | 0.11 | 0.11 | — | — | — | — |
| (II-2-PSMAt1) | — | — | 0.11 | 0.12 | — | — |
| (II-11- | — | — | — | — | 0.11 | 0.12 |
| PSMAt1) | | | | | | |
| SnCl₂•2H₂O | 0.11 | 0.03 | 0.11 | 0.03 | 0.11 | 0.03 |
| Sodium | 1.15 | 0.26 | 1.15 | 0.26 | 1.15 | 0.26 |
| tartrate | | | | | | |
| NaHCO₃ | 10.71 | 0.90 | 10.71 | 0.90 | 10.71 | 0.90 |

The kits may be scaled to, for example, two or three times the amounts shown in the table above.
Radiolabelling of (II-1-PSMAt1), (II-2-PSMAt1), or (II-11-PSMAt1) with ^{99m}TcO₄⁻

(II-1-PSMAt1) or (II-2-PSMAt1) were radiolabelled with generator-produced ^{99m}TcO₄⁺ in saline solution (0.9% NaCl in water, w/v), using the lyophilised kits described. The radiolabelling reaction mixtures were either left to react at ambient temperature (˜22° C.) for 5 min, or heated at 100° C. for 5 min. Aliquots were analysed by iTLC and analytical C₁₈-HPLC to determine radiochemical yields. The species attributed as (Tc-III-1-PSMAt1) eluted at 11.0-12.5 min; (Tc-III-2-PSMAt1) eluted at 12.5-14.0 min. Analytical HPLC conditions: 20 min, 5% min⁻¹ linear increase from 100% A to 100% B (flow rate of 1 ml/min, A=water containing 0.1% TFA, B=acetonitrile containing 0.1% TFA, analytical (4.6×150 mm, 5 μm) Agilent Zorbax Eclipse XDB-C18 column).

(II-11-PSMAt1) was radiolabelled with generator-produced ^{99m}TcO₄⁻ (200 MBq, 300 uL) in saline solution (0.9% NaCl in water, w/v), using the lyophilised kit described above. The radiolabelling reaction mixture was heated at 100° C. for 5 min. Aliquots were analysed by iTLC and analytical C₁₈-HPLC to determine radiochemical yields. The species attributed as (Tc-III-11-PSMAt1) eluted at 9.7-11.7 min. Analytical HPLC conditions: 20 min, 5% min⁻¹ linear increase from 100% A to 100% B (flow rate of 1 ml/min, A=water containing 0.1% TFA, B=acetonitrile containing 0.1% TFA, analytical (4.6×150 mm, 5 μm) Agilent Zorbax Eclipse XDB-C18 column).

Two separate iTLC analyses were undertaken, to enable quantification of ^{99m}Tc-colloids, unreacted ^{99m}TcO₄⁻ and the complex.

To quantify amounts of unreacted ^{99m}TcO₄⁻, acetone was used as a mobile phase: R_{f} values: ^{99m}TcO₄⁻>0.9, ^{99m}Tc colloids<0.1, complex <0.1.

To quantify ^{99m}Tc-colloid formation, a 1:1 mixture of methanol and 2M aqueous ammonium acetate solution was used as a mobile phase: ^{99m}TcO₄⁻>0.9, ^{99m}Tc colloids<0.1, the complex >0.9.

For in vitro and in vivo studies, these kit-based reaction solutions were further purified. Solutions of either (Tc-III-1-PSMAt1), (Tc-III-2-PSMAt1), or (Tc-III-11-PSMAt1) prepared from kits were applied to a SE-HPLC column, using an aqueous mobile phase of phosphate buffered saline. Fractions containing either (Tc-III-1-PSMAt1), (Tc-III-2-PSMAt1), or (Tc-III-11-PSMAt1) (>95% radiochemical purity) eluted at 10-12 mins. Other reaction components, including unreacted starting materials and impurities also eluted at distinct retention times: unlabelled (II-1-PSMAt1) ligand eluted at 16-17 min, unlabelled (II-2-PSMAt1) eluted at 27-28 min, ^{99m}TcO₄⁻ eluted at 14-15 min and ^{99m}Tc-colloid was trapped on the column.

### Preparation of Compounds (^{99g}Tc-III-1-PSMAt1) and (^{99g}Tc-III-2-PSMAt1)

The ^{99g}Tc(V) precursor N^{t}Bu₄[^{99g}TcOCl₄] was prepared following a previously described method (A. Davison, C. Orvig, H. S. Trop, M. Sohn, B. V. Depamphilis and A. G. Jones, Inorg. Chem., 1980, 19, 1988-1992). A solution of either (II-1-PSMAt1) or (II-2-PSMAt1) (1.0 mg, ˜1 μmol, 2 equiv.) dissolved in methanol (300 μL, degassed) was combined with a solution of N^{t}Bu₄[^{99g}TcOCl₄](0.25 mg, 0.46 μmol, 1 equiv.) in methanol (50 μL). The resulting pale yellow solution was left to react at ambient temperature for 15 min.

(^{99g}Tc-III-1-PSMAt1): HR-MS-ESI m/z: [M+H]²⁺ 1098.8183 (calculated for C₁₀₂H₁₂₅N₈O₃₂P₄Tc 1098.8221 (100% abundance peak)), [M+Na]²⁺ 1109.8091 (calculated for C₁₀₂H₁₂₄N₈O₂P₄TcNa 1109.8130 (100% abundance peak)); LR-MS-ESI m/z: [M+H]²⁺ 1099.0 (calculated for C₁₀₂H₁₂₅N₈O₃₂P₄Tc 1098.5), [M+Na]²⁺ 1110.0 (calculated for C₁₀₂H₁₂₄N₈O₃₂P₄TcNa 1109.5), [M+K]²⁺ 1117.7 (calculated for C₁₀₂H₁₂₄N₈O₃₂P₄TcK 1117.5), [M+2H]³⁺ 732.7 (calculated for C₁₀₂H₁₂₆N₈O₃₂P₄Tc 732.7), [M+H+K]³⁺ 745.2 (calculated for C₁₀₂H₁₂₆N₈O₃₂P₄TcK 745.3).

(^{99g}Tc-III-2-PSMAt1): HR-MS-ESI m/z: [M+H]²⁺ 1154.8811 (calculated for C₁₁₀H₁₄₁N₈O₃₂P₄Tc 1154.8847 (100% abundance peak)), [M+Na]²⁺ 1165.8718 (calculated for C₁₁₀H₁₄₀N₈O₃₂P₄TcNa 1165.8756 (100% abundance peak)); LR-MS-ESI m/z: [M+H]²⁺ 1155.0 (calculated for C₁₁₀H₁₄₁N₈O₃₂P₄Tc 1154.5), [M+Na]²⁺ 1165.8 (calculated for C₁₁₀H₁₄₀N₈O₃₂P₄TcNa 1165.5), [M+K]²⁺ 1173.8 (calculated for C₁₁₀H₁₄₀N₈O₃₂P₄TcK 1173.5), [M+2H]³⁺ 770.3 (calculated for C₁₀₀H₁₄₂N₈O₃₂P₄Tc 770.0), [M+H+K]³⁺ 782.8 (calculated for C₁₁₀H₁₄₁N₈O₃₂P₄TcK 782.7).

### Example 9—Biological evaluation of (Tc-III-1-PSMAt1), (Tc-III-2-PSMAt1), and (Tc-III-11-PSMAt1)

Compound (Tc-III-1-PSMAt1)/[^{99m}TcO₂(II-1-PSMAt1)₂]⁺ and Compound (Tc-III-2-PSMAt1)/[^{99m}TcO₂(II-2-PSMAt1)₂]⁺ were isolated and purified to evaluate stability, affinity for PSMA in vitro and in vivo and pharmacokinetics.

TABLE 6

Dissociated ^{99m}Tc (measured by analytical HPLC)

| | Compound (Tc-III-1-PSMAt1) | Compound (Tc-III-2-PSMAt1) |
|---|---|---|
| Time | ([^{99m}TcO₂(II-1-PSMAt1)₂]⁺) | ([^{99m}TCO₂(II-2-PSMAt1)₂]⁺) |

| | | | |
|---|---|---|---|
| 1 | h | 0% | 0.1% |
| 4 | h | 0.7% | 1.6% |
| 24 | h | 4.2% | 6.5% |

Table 6 shows the amount of dissociated ^{99m}Tc after incubation of Compound (Tc-III-1-PSMAt1) and Compound (Tc-III-2-PSMAt1) in serum.

The stability of Compound (Tc-III-1-PSMAt1) and Compound (Tc-III-2-PSMAt1) were assessed in serum over 24 hours. Both tracers exhibit high stability, with over 90% intact over 24 hours, as determined by analytical C18 radio-HPLC. With the exception of “free” ^{99m}Tc, no other degradation products are observed in HPLC chromatograms. The log D_{OCT/PBS} of (Tc-III-1-PSMAt1) is −2.45 and the log D_{OCT/PBS} of (Tc-III-2-PSMAt1) is −2.08, suggesting that both are hydrophilic and are likely to clear via a renal pathway.

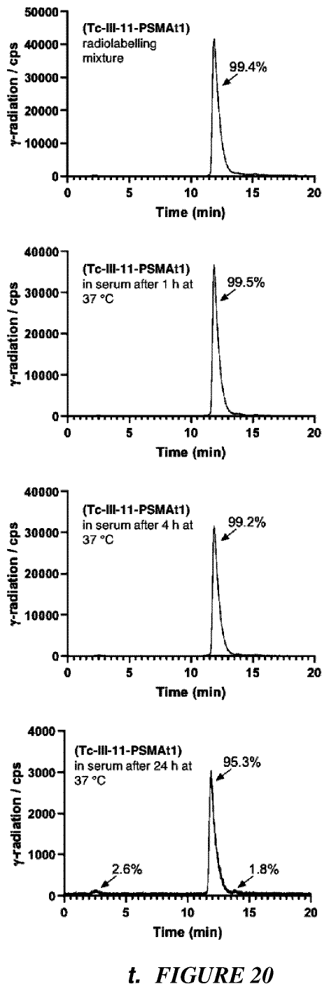

A solution containing Compound (Tc-III-11-PSMAt1) (20 μL, 13 MBq) was added to filtered human serum (180 μL) and incubated at 37° C. At 1, 4 and 24 h, samples were taken and treated with an equal volume of ice-cold acetonitrile to precipitate and remove serum proteins. Acetonitrile in the supernatant was then removed by evaporation under a stream of N₂ gas. The final solution was then analysed by reverse-phase analytical HPLC (FIG. 20). Compound (Tc-III-11-PSMAt1) exhibits high stability, with over 95% intact over 24 hours, as determined by analytical C18 radio-HPLC.

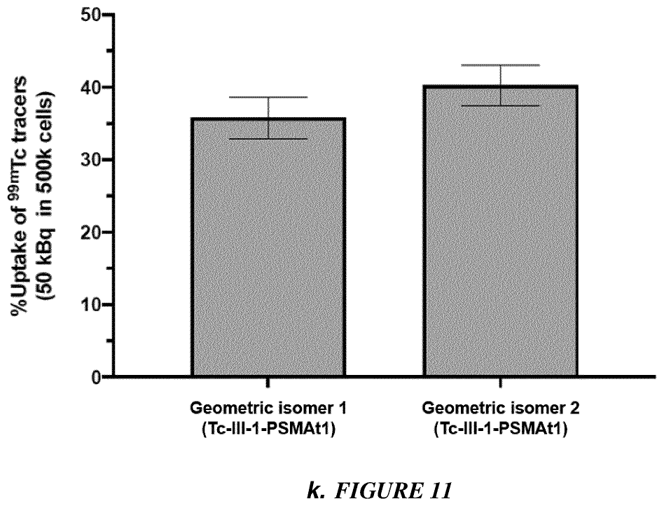

^{99m}Tc-DP-peptide radiotracers contain two different isomers. Such isomers are known as “geometric cis/trans” isomers. To show that the isomers have equivalent biological behaviour, the “cis” and “trans” geometric isomers of (Tc-III-1-PSMAt1) were separated: both have near identical uptake in PSMA-positive cells (FIG. 11).

(Tc-III-1-PSMAt1) and (Tc-III-2-PSMAt1) uptake in DU145, DU145-PSMA, LNCaP, and PC-3 cells

The following experiment was performed in biological triplicate.

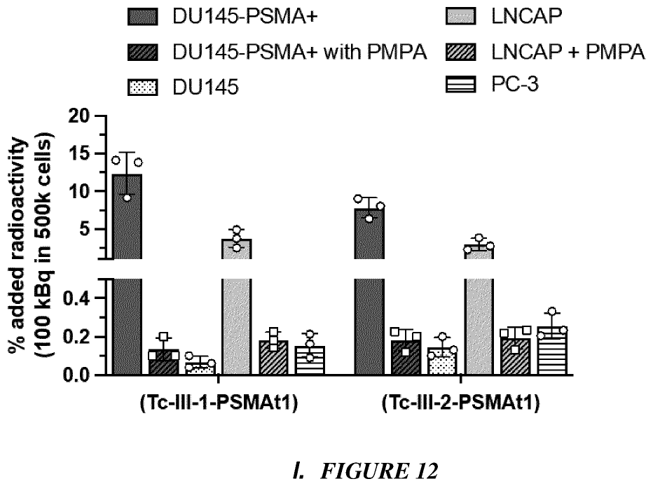

A panel of cell lines were selected that either expressed GCP(II)/PSMA (DU145-PSMA (genetically modified to express PSMA) (see F. Kampmeier, J. D. Williams, J. Maher, G. E. Mullen and P. J. Blower, EJNMMI Res., 2014, 4, 13), and LNCaP (CRL-1740)), or had low GCP(II)/PSMA expression (DU145 (HTB-81) and PC-3 (CRL-1435)). All cell lines were cultured in RPMI 1640 medium (R0883, Sigma) containing 10% foetal bovine serum, 2 mM L-glutamine, and 100 U·mL⁻¹ penicillin and 100 μg·mL⁻¹ streptomycin, except for PC-3 cells which were cultured in low-glucose Dulbecco's Modified Eagle Medium (DMEM, D5546, Sigma) supplemented as above. Cells were maintained at 37° C. and 5% CO₂. Cells were seeded in 6-well plates at a density of 5×10⁵ cells per well in 2 mL complete media to achieve 70-80% confluency the following day. Prior to treating cells, cell medium (1 mL/well) was replaced. Solutions containing either (Tc-III-1-PSMAt1) or (Tc-III-2-PSMAt1) (100 kBq, in 5-12 μL of phosphate buffered saline, >95% radiochemical purity) were added to each well, and the cells incubated at 37° C. for 1 h. Uptake studies were also performed after a 2 min incubation with the PSMA inhibitor 2-(phosphonomethyl)pentane-1,5-dioic acid (PMPA; 30 μL of 750 μM PMPA solution/well). After 60 min incubation, the plates were placed on ice, the supernatant was removed and the cells were washed with ice cold phosphate buffered saline solution (3×0.5 mL). The cells were lysed with ice cold radioimmunoprecipitation assay buffer (RIPA buffer, 500 μL; 150 mM sodium chloride, 0.1% w/w sodium dodecyl sulfate (SDS), 0.5% w/w sodium deoxycholate (NaDOC), 1% w/w Triton-X) and samples were collected for radioactivity counting. Results in FIG. 12 are depicted as means±SD of independent biological experiments (performed on different days with different radiotracer preparations).

(Tc-III-1-PSMAt1) and (Tc-III-2-PSMAt1) exhibited uptake in DU145-PSMA+ cells (12.4±2.8% AR [percentage added radioactivity], and 7.8±1.3% AR respectively). This uptake was specific: DU145-PSMA+ cell uptake of (Tc-III-1-PSMAt1) and (Tc-III-2-PSMAt1) could be blocked with PMPA, and there was negligible uptake in parental DU145 cells (FIG. 12).

In LNCaP cells, uptake of (Tc-III-1-PSMAt1) and (Tc-III-2-PSMAt1) measured 3.7±1.2% AR and 3.0±0.8% AR respectively, whilst uptake of both tracers in PC3 cells measured less than 0.3% AR. Uptake in LNCaP cells could also be blocked with PMPA (FIG. 12).

### In Vitro Time Course and Localisation of (Tc-III-1-PSMAt1) and (Tc-III-2-PSMAt1)

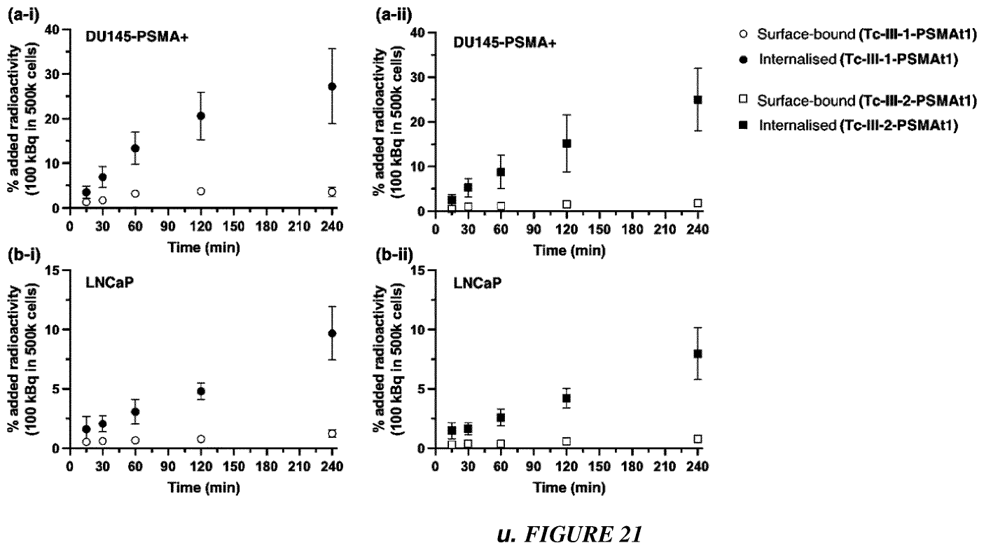

To determine the cellular uptake and localisation of each tracer over time, DU145-PSMA and LNCAP cells were seeded as above. Cells were replenished with complete medium (1 mL) 1 h prior to the addition of either (Tc-III-1-PSMAt1) or (Tc-III-2-PSMAt1) (100 kBq, in 5-7 L of phosphate buffered saline, >95% radiochemical purity). Cells were incubated at 37° C. under 5% CO₂ with three technical replicates for each condition. Following 15, 30, 60 and 120 min incubation, the supernatant was collected and cells washed three times with PBS (1 mL) to determine the unbound fraction, followed by an acid wash (0.5 M glycine, pH 2.5) to determine cell surface-bound activity. Cells were then lysed with cold RIPA buffer (500 μl) to determine activity internalised by the cells. Radioactivity content was determined by gamma-counter. Results are depicted in FIG. 21 as means±SD of independent biological experiments (performed on different days with different radiotracer preparations).

Uptake of both radiotracers increased over 2 hours, and the majority of ^{99m}Tc-cell associated radioactivity was present in the internalised cell fraction at all measured time points, suggesting that (Tc-III-1-PSMAt1) and (Tc-III-2-PSMAt1) are rapidly internalised after PSMA binding, for both PSMA-expressing cell lines. (Tc-III-1-PSMAt1) uptake (both surface-bound and internalised radioactivity) was slightly higher than that for (Tc-III-2-PSMAt1).

### In Vivo Imaging of (Tc-III-1-PSMAt1) and (Tc-III-2-PSMAt1) in Healthy Mice

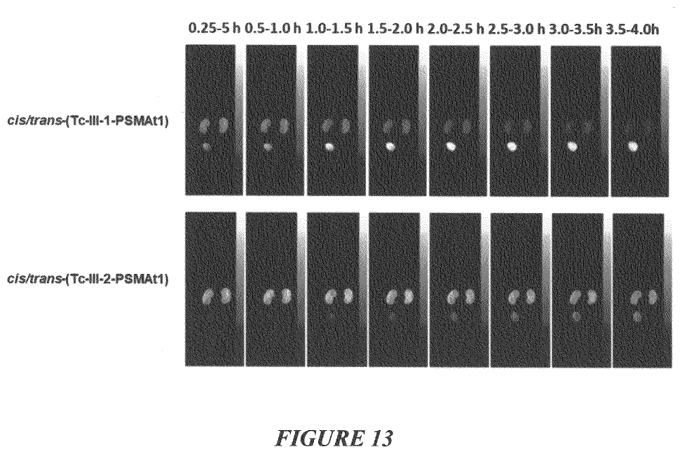

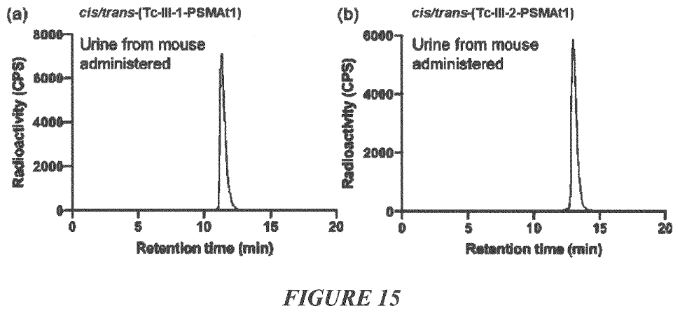

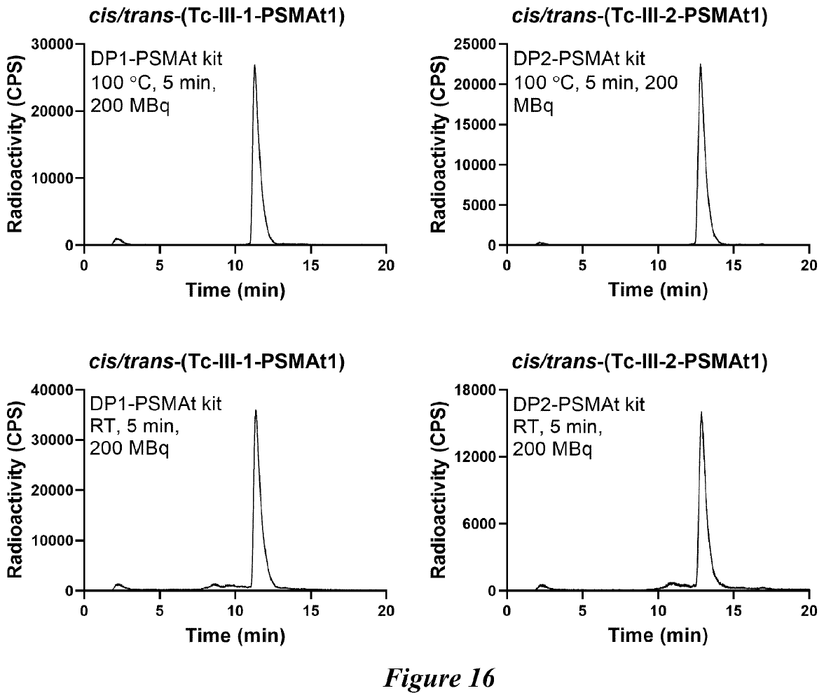

Animal imaging studies were ethically reviewed and carried out in accordance with the Animals (Scientific Procedures) Act 1986 (ASPA) UK Home Office regulations governing animal experimentation. Mice were purchased from Charles River (Margate, UK). A male SCID-beige mouse (approx. 3 months old, n=1) was anaesthetised (2.5% v/v isofluorane, 0.8-1.0 L/min 02 flow rate) and injected intravenously via the tail vein with (Tc-III-1-PSMAt1) (100 μL, 26 MBq, >99% RCP, 0-5 μg PSMAt peptide in phosphate buffered saline) or (Tc-III-2-PSMAt1) (160 μL, 30 MBq, >99% RCP, 0-5 μg PSMAt peptide in phosphate buffered saline), followed immediately by CT acquisition, and SPECT scanning. SPECT/CT imaging was accomplished using a pre-clinical nanoScan SPECT/CT Silver Upgrade instrument (Mediso), calibrated for technetium-99m (FIG. 13). The SPECT scans were acquired by helical SPECT (4-head scanner with 4×9 pinhole collimators), and CT scans by helical CT (55 kVP X-ray source, 1000 ms exposure time in 180 projections over 9 min). 1.0 mm pinhole collimators were used. SPECT acquisition was done in eight segments: the first segment was acquired at 15-30 min post injection (frame time of 12s; 9 min acquisition time), followed by seven imaging segments of 30 min each (frame time of 33s; 24.75 min acquisition time) up until 4 h post-injection. At the end of the imaging procedure, the mouse was culled by cervical dislocation and a sample of the urine analysed by analytical HPLC (FIG. 15). SPECT images were reconstructed at 0.3 mm isotropic voxel size with the HiSPECT (Scivis GmbH) reconstruction software package using standard reconstruction with 35% smoothing and 9 iterations. The CT and SPECT images were further processed and analysed using VivoQuant software (inviCRO, USA).

### Biodistribution of (Tc-III-1-PSMAt1) and (Tc-III-2-PSMAt1) in Healthy Mice

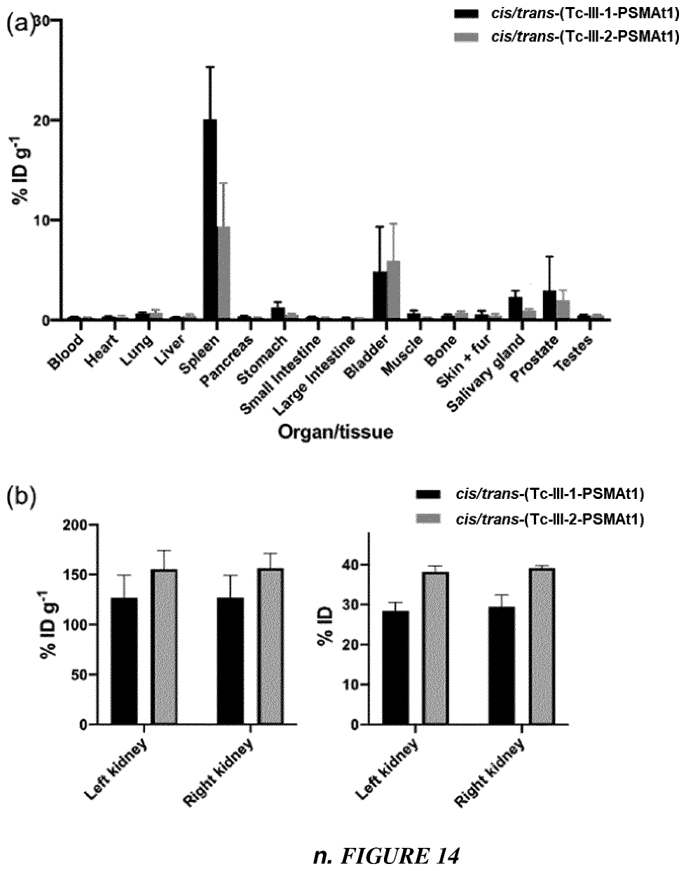

Male SCID-beige mice (approx. 3 months old) were weighed, anaesthetised (2.0-2.5% v/v isofluorane, 1.0-1.0-1.5 L/min 02 flow rate) and injected with (Tc-III-1-PSMAt1) solution (50 μL, approx. 13 MBq in phosphate buffered saline, n=4) or (Tc-III-2-PSMAt1) solution (80 μL, approx. 15 MBq in phosphate buffered saline, n=4) by intravenous tail vein injection. The mice were kept under anaesthesia until they were culled by cervical dislocation 2 h post-injection. The biodistribution of the tracer was assessed by dissecting, weighing and gamma counting organs/tissues, alongside standard solutions of known ^{99m}Tc radioactivity. The radioactivity measured for each organ/tissue was normalised to obtain values of percentage injected dose per gram (% ID/g) (FIG. 14).

### Example 10—Biodistribution of Compound (Tc-III-1-PSMAt1)/[^{99m}TcO₂(DP^{Ph}-PSMAt)₂]⁺ and Compound (Tc-III-2-PSMAt1)/[^{99m}TcO₂(DP^{Tol}-PSMAt) )₂+ in Mice Bearing Prostate Cancer Tumours

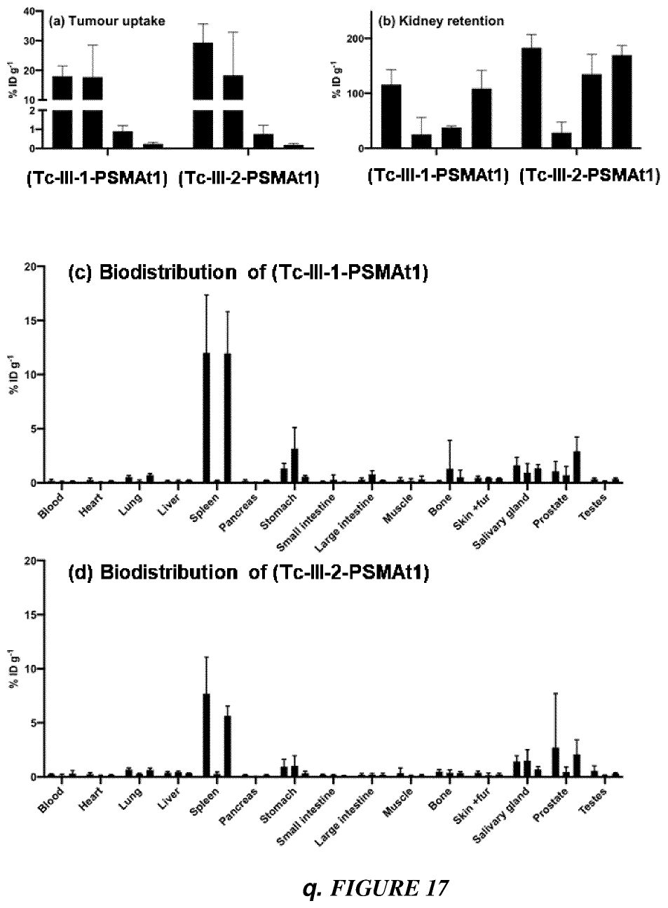

The biodistributions of (Tc-III-1-PSMAt1) and (Tc-III-2-PSMAt1) were assessed in SCID/Beige mice bearing DU145-PSMA+ tumours (FIG. 17a). Each animal was administered either (Tc-III-1-PSMAt1) or (Tc-III-2-PSMAt1), and euthanized at either 2 h or 24 h post-injection, followed by organ harvesting for ex vivo radioactivity counting. Higher amounts of (Tc-III-2-PSMAt1) were measured in tumours 2 h post-injection (29.4±6.3% ID g⁻¹, [percentage injected dose per gram]) compared to (Tc-III-1-PSMAt1) (18.0±3.5% ID g⁻¹, mean difference=29.3% ID g⁻¹, p=0.008). In other organs, concentrations of radioactivity were similar to that observed in healthy SCID/Beige mice. At 24 h post-injection, significant amounts of ^{99m}Tc radioactivity were still present in tumours—in fact, there was no statistically significant difference between ^{99m}Tc radioactivity concentration in tumours at 2 h and 24 h post-injection, for animals administered the same tracer (FIG. 17a).

To assess specificity of each radiotracer, separate groups of animals, also bearing DU145-PSMA+ tumours, were co-administered either (Tc-III-1-PSMAt1) and PMPA, or (Tc-III-2-PSMAt1) and PMPA, to inhibit PSMA-mediated uptake of radiotracer. Additionally, groups of mice bearing parental DU145 tumours (that do not express PSMA) were also administered these ^{99m}Tc radiotracers. Animals were also euthanised 2 h post-injection, followed by organ harvesting for ex vivo radioactivity counting (FIG. 17).

In mice bearing DU145-PSMA+ tumours, co-administration of PMPA substantially decreased uptake of both (Tc-III-1-PSMAt1) or (Tc-III-2-PSMAt1) in tumours (FIG. 17a). For (Tc-III-1-PSMAt1), co-administration decreased uptake to 0.91±0.29% ID g⁻¹ in the tumour (compared to administration of (Tc-III-1-PSMAt1) only: mean difference=17.12% ID g⁻¹, p=4×10⁻⁶). For (Tc-III-2-PSMAt1), co-administration decreased uptake to 0.76±0.45% ID g⁻¹ in the tumour (compared to administration of (Tc-III-2-PSMAt1) only: mean difference=28.62% ID g⁻¹, p=7×10⁻⁶). Similarly, for animals bearing DU145 tumours that do not express PSMA, tumour uptake of (Tc-III-1-PSMAt1) decreased to 0.24±0.07% ID g⁻¹, and tumour uptake of (Tc-III-2-PSMAt1) decreased to 0.18±0.07% ID g⁻¹ (FIG. 17a) For both (Tc-III-1-PSMAt1) and (Tc-III-2-PSMAt1), co-administration of PMPA significantly decreased uptake in the spleen (FIG. 17c,d).

For both radiotracers, the concentration of ^{99m}Tc radioactivity in kidneys 2 h post-injection was high (FIG. 17b). Notably, for animals administered (Tc-III-1-PSMAt1), co-administration of PMPA significantly decreased retention of ^{99m}Tc radioactivity in kidneys. In contrast, although co-administration of PMPA also decreased radioactivity concentration in the kidneys for animals injected with (Tc-III-2-PSMAt1), this effect was much less pronounced.

### SPECT/CT Imaging

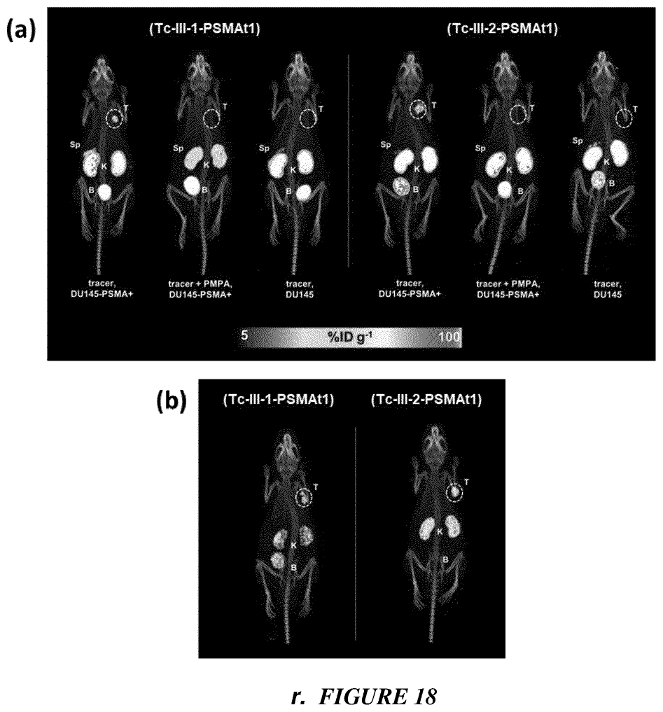

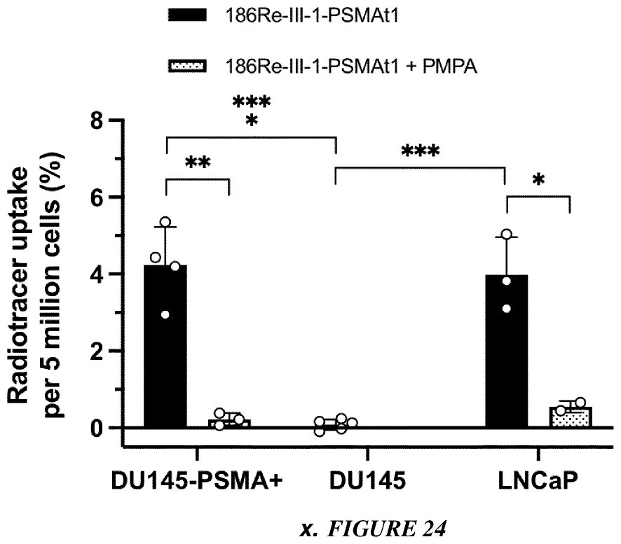

In SPECT/CT scans of animals administered either (Tc-III-1-PSMAt1) or (Tc-III-2-PSMAt1) only, tumours could be clearly delineated at both 2 h (FIG. 18a) and 24 h post-injection (FIG. 18b). The kidneys and bladder were also clearly visible across these timepoints, consistent with prior data showing that the radiotracers are excreted via a renal pathway, and ex vivo biodistribution data. SPECT/CT also showed negligible tumour uptake for (i) animals either co-administered PMPA or (ii) animals bearing DU145 tumours that do not express PSMA receptor (FIG. 18a). For animals administered either (Tc-III-1-PSMAt1) or (Tc-III-2-PSMAt1), the spleen was also identified in SPECT/CT acquired at 2 h post-injection. Co-administration of PMPA decreased spleen uptake of both radiotracers.

Preparation of tumour-bearing mice: The GCP(II)/PSMA-negative cell line used in these experiments was DU145, a human carcinoma prostate cancer cell line derived from a brain metastatic site. The GCP(II)/PSMA-expressing cell line used in these experiments was a genetically modified daughter cell line of DU145, DU145-PSMA+. This cell line had previously been transduced to express full-length human GCP(II)/PSMA, following F. Kampmeier, J. D. Williams, J. Maher, G. E. Mullen and P. J. Blower, EJNMMI Res., 2014, 4, 13. These cells were cultured in DMEM medium supplemented with 10% foetal bovine serum, 2 mM L-glutamine, and penicillin/streptomycin. To prepare for experiments, cells were grown at 37° C. in an incubator with humidified air equilibrated with 5% CO₂.

Animal studies complied with the guidelines on responsibility in the use of animals in bioscience research of the U.K. Research Councils and Medical Research Charities, under U.K. Home Office project and personal licences. Subcutaneous prostate cancer xenografts were produced in SCID/beige mice (male, 7-12 weeks old) by injecting 4×10⁶ DU145-PSMA or DU145 cells suspended in PBS (100 μL) on the right shoulder. Imaging was performed once a tumour had reached 5-10 mm in diameter (3-4 weeks after injection). For imaging purposes, the mice were anaesthetised, positioned on the scanner, and tail vein cannulated. For biodistribution purpose, the mice were anaesthetised, the radiotracers were injected via the tail vein.

SPECT/CT scanning: SPECT/CT scans were acquired on a dedicated small animal SPECT system, NanoSPECT/CT Silver Upgrade (Mediso Ltd., Budapest, Hungary), calibrated for ^{99m}Tc. The animals (2 mice per group) were cannulated via tail vein, the radiotracers (10-26 MBq) were administered while the animals were on the scanner followed by a helical CT scan (45 kVP X-ray source, 1000 ms exposure time in 180 projections over 7.5 min). After 15 min post-injection, whole body SPECT scans were acquired (30 min×4, conducted sequentially) with a frame time of 33 s (using a 4-head scanner with 4×9 [1.4 mm] pinhole collimators in helical scanning mode). After this, animals were allowed to recover, culled at 24 h post-injection (by cervical dislocation and tail-vein nick to confirm death), organs/tissues harvested, weighed and radioactivity counted using a gamma counter. For each radiotracer, an additional animal was administered tracer and recovered, before being anaesthetised and undergoing SPECT/CT scanning at 24 h post-injection, followed by culling and ex vivo tissue counting. SPECT/CT images were reconstructed in a 256×256 matrix using HiSPECT (ScivisGmbH), a reconstruction software package and visualised and quantified using VivoQuant VivoQuant v.3.5 software (InVicro LLC., Boston, USA).

Biodistribution studies: The ^{99m}Tc radiotracers (7-18 MBq) were administered via tail vein injection under isoflurane anaesthesia (5 mice per group). The animals were allowed to recover, roaming free in a gridded cage. The animals were euthanised by cervical dislocation 2 h post-injection, organs/tissues harvested, weighed and radioactivity counted using a gamma counter. Data were analysed in GraphPad Prism 9 (version 9.1.1) and expressed as mean±standard deviation (SD). Student t tests were used to determine statistical significance.

### Example 11—Preparation and Characterisation of (^{99g}Tc-III-11-PSMAt1)

To a sample of Compound (II-11-PSMAt1) (˜1 mg) dissolved in DMF was added N^{t}Bu₄ [^{99g}TcOCl₄](˜0.3 mg). The solution was analysed.

LC-MS (ESI, positive mode, low resolution) Retention time=8:04-8:17 min LRMS: [M+H]²⁺ 1220 (observed), 1219 (calculated).

### Example 12—Preparation and Characterisation of Compound (⁶⁴Cu-III-1-PSMAt1)/[⁶⁴Cu(II-1-PSMAt1)₂+ and Compound (⁶⁴Cu-III-2-PSMAt1)/[⁶⁴Cu(II-2-PSMAt1)₂]⁺

### ⁶⁴Cu Radiolabelling of (II-1-PSMAt1) and (II-2-PSMAt1)

⁶⁴Cu was produced by ⁶⁴Ni(p,n)⁶⁴Cu nuclear reaction on a CTI RDS 112 11 MeV cyclotron and purified to give ⁶⁴Cu²⁺ in 0.1 M HCl solutions used for radiolabelling (see M. S. Cooper, M. T. Ma, K. Sunassee, K. P. Shaw, J. D. Williams, R. L. Paul, P. S. Donnelly and P. J. Blower, Bioconjug. Chem., 2012, 23, 1029-1039). The ⁶⁴Cu²⁺ solutions (in 0.1 M HCl) were dried under a flow of nitrogen with heating at 100° C., and the residue re-dissolved in ammonium acetate solution (0.1 M, pH 7). An aliquot of ammonium acetate solution containing ⁶⁴Cu²⁺ (10 MBq, 50-100 μL) was added to either (II-1-PSMAt1) (50 μg) or (II-2-PSMAt1) (50 μg) dissolved in aqueous ammonium acetate (0.1 M), to give a final radiolabelling solution of 200 μL volume. The radiolabelling mixtures were left to react at ambient temperature (˜22° C.) for 20 min. Aliquots were analysed by iTLC and analytical HPLC to determine radiochemical yield. By Cis-analytical HPLC, the species attributed as (⁶⁴Cu-III-1-PSMAt1) eluted at 12.0-13.0 min; (⁶⁴Cu-III-2-PSMAt1) eluted at 13.5-14.5 min; unreacted ⁶⁴Cu²⁺ eluted with the solvent front at 2.0-3.5 min.

iTLC analysis was undertaken to enable quantification of unreacted ⁶⁴Cu²⁺ and the complex. Citrate buffer (0.1 M, pH 5) was used as a mobile phase: R_{f} values: unreacted ⁶⁴Cu²⁺>0.9, complex <0.1.

Log D_{OCT/PBS} D of (⁶⁴Cu-III-1-PSMAt1) and (⁶⁴Cu-III-2-PSMAt1)

The following procedure was carried out in triplicate. A solution containing either (⁶⁴Cu-III-1-PSMAt1) or (⁶⁴Cu-III-2-PSMAt1) (0.5 MBq in 20 μL) was combined with phosphate buffered saline (pH 7.4, 480 μL) and octanol (500 μL), and the mixture was agitated for 30 min. The mixture was then centrifuged (10 000 rpm, 10 min), and aliquots of octanol and aqueous phosphate buffered saline were analysed for radioactive using a gamma counter. log D_{OCT/PBS} (⁶⁴Cu-III-1-PSMAt1): −3.30±0.03; log D_{OCT/PBS} (⁶⁴Cu-III-2-PSMAt1): −3.01±0.06.

Serum Stability of (⁶⁴Cu-III-1-PSMAt1) and (⁶⁴Cu-III-2-PSMAt1)

A sample of (⁶⁴Cu-III-1-PSMAt1) (>99.0% RCP, 1.7 MBq, 5 g DP^{Ph}-PSMAt ligand) or (⁶⁴Cu-III-2-PSMAt1) (>99.0% RCP, 1.7 MBq, 5 μg DP^{Tol}-PSMAt ligand) in an aqueous solution of ammonium acetate (20 μL, 0.1 M) was added to filtered human serum from a healthy volunteer (180 μL), and incubated at 37° C. At 1, 4 and 24 h, aliquots were taken. Each aliquot (300 μL) was treated with ice-cold acetonitrile (300 μL) to precipitate and remove serum proteins. Acetonitrile in the supernatant was then removed by evaporation under a stream of N₂ gas (40° C., 30 min). The final solution was then analysed by reverse-phase analytical HPLC (method 2). Radiochromatograms of serum samples showed that (⁶⁴Cu-III-1-PSMAt1) and (⁶⁴Cu-III-2-PSMAt1) were still present, even after 24 h incubation in serum, with no other degradation products detectable.

### Preparation of (^{nat}Cu-III-1-PSMAt1) and (^{nat}Cu-III-2-PSMAt1)

A solution of either (II-1-PSMAt1) or (II-2-PSMAt1) (1.0 mg, ˜1 μmol, 2 equiv.) in saline (500 μL) was added to a solution of [Cu^{I}(MeCN)₄] PF₆ (170-180 μg, ˜0.5 μmol, 1 equiv.) in acetonitrile (dry, deoxygenated, 500 μL). The reaction mixture was left to react at ambient temperature for 60 min. The product was isolated by semi-preparative HPLC (method 6), lyophilising the product fractions eluting at either −46-47 min ((^{nat}Cu-III-1-PSMAt1)) or 56-57 min ((⁶⁴Cu-III-2-PSMAt1)). Yield=30-40%.

(^{nat}Cu-III-1-PSMAt1): HR-MS-ESI m/z: [M+H]²⁺ 1064.3338 (calculated for C₁₀₂H₁₂₅O₃₀N₈P₄Cu 1064.3369); LR-MS-ESI+m/z: [M+H]²⁺ 1065.8 (calculated for C₁₀₂H₁₂₅O₃₀N₈P₄Cu 1065.3), [M+Na]²⁺ 1077.2 (calculated for C₁₀₂H₁₂₄O₃₀N₈P₄CuNa 1076.3), [M+K]²⁺ 1084.6 (calculated for C₁₀₂H₁₂₄O₃₀N₈P₄CuK 1084.3), [M+2H]³⁺ 711.0 (calculated for C₁₀₂H₁₂₆O₃₀N₈P₄Cu 710.5).

(^{nat}Cu-III-2-PSMAt1): HR-MS-ESI m/z: [M+H]²⁺ 1120.3973 (calculated for C₁₀₀H₁₄₁O₃₀N₈P₄Cu 1120.3995); LR-MS-ESI+m/z: [M+H]²⁺ 1121.3 (calculated for C₁₁₀H₁₄₁O₃₀N₈P₄Cu 1121.4), [M+Na]²⁺ 1132.6 (calculated for C₁₁₀H₁₄₀O₃₀N₈P₄CuNa 1132.4), [M+2H]³⁺ 748.0 (calculated for C₁₀₀H₁₄₂O₃₀N₈P₄Cu 747.9), [M+H+K]³⁺ 761.3 (calculated for C₁₁₀H₁₄₁O₃₀N₈P₄CuK 760.6).

### Example 13—Preparation and Characterisation of Compound (^{nat}Re-III-1-PSMAt1)/[^{nat}ReO₂(II-1-PSMAt1)₂]⁺ and Compound (^{nat}Re-III-2-PSMAt1)/[^{nat}ReO₂(II-2-PSMAt1)₂]⁺

### Preparation of (^{nat}Re-III-1-PSMAt1) and (^{nat}Re-III-2-PSMAt1)

A solution of either (II-1-PSMAt1) (˜5.1 mg, 4.9 μmol, 1 equiv.) or (II-2-PSMAt1) (2.6 mg, 2.4 gmol, 1 equiv.) and DIPEA (6 μL) in DMF was combined with a solution of [^{nat}ReO₂I(PPh₃)₂](˜2.2 mg, ˜2.5 μmol, 0.5 or 1 equiv., respectively) in DMF. The resulting dark brown solution was left to react at room temperature for 2-3 h. The reaction solution was applied to a reverse phase C₁₈ semi-preparative HPLC column, and purified by HPLC (C₁₈ semi-preparative HPLC (9.4×250 mm, 5 μm) Agilent Zorbax Eclipse XDB-C18 column: 90 min, isocratic flow at 95% A for 5 min, then 0.93% min⁻¹ linear increase from 95% A/5% B to 25% A/75% B, followed by 2.5% min⁻¹ linear increase from 25% A to 0% A, flow rate of 3 mL min⁻¹; A=water with 0.005% acetic acid, B=acetonitrile with 0.005% acetic acid; Detection at 214 and 254 nm). The fractions containing the desired product were lyophilised to yield (^{nat}Re-III-1-PSMAt1) (1-2 mg, 0.4-0.8 μmol, 15-30% yield) and (^{nat}Re-III-2-PSMAt1) (-1-1.5 mg, −0.5 μmol, ˜20% yield) as solids.

Using a relatively “long” HPLC method (gradient mobile phase for 60 min; 1 ml min⁻¹ flow rate; 1% min⁻¹ linear increase from 100% A/0% B to 40% A/60% B; A=water containing 0.1% TFA, B=acetonitrile containing 0.1% TFA, analytical (4.6×150 mm, 5 μm) Agilent Zorbax Eclipse XDB-C18 column) to separate out cis and trans isomers, the species attributed as (^{nat}Re-III-1-PSMAt1) eluted at 38.11 and 38.51 min; (^{nat}Re-III-2-PSMAt1) eluted at 45.37 and 46.07 min.

(^{nat}Re-III-1-PSMAt1): HR-MS-ESI m/z: [M+2H]³⁺ 761.9001 (calculated for C₁₀₂H₁₂₆O₃₂N₈P₄Re 761.8990); [M+H+Na]³⁺ 769.2274 (calculated for C₁₀₂H₁₂₅O₃₂N₈P₄ReNa 769.2263). (^{nat}Re-III-2-PSMAt1): HR-MS-ESI m/z: [M+2H]³⁺ 799.2757 (calculated for C₁₀₀H₁₄₂O₃₂N₈P₄Re 799.2741); [M+H+Na]³⁺ 806.6023 (calculated for C₁₁₀H₁₄₁O₃₂N₈P₄ReNa 806.6014).

### Example 14—Preparation and Use of Radiolabelling Kits and Effect on Radiochemical Yield

To assess the feasibility of ^{99m}Tc radiolabelling of (II-1-RGD), (II-1-PSMAt1), (II-2-PSMAt1), and (II-11-PSMAt1) with a “kit” formulation, lyophilised mixtures of (II-1-RGD), (II-1-PSMAt1), (II-2-PSMAt1), or (II-11-PSMAt1), tin(II) chloride, sodium bicarbonate, and sodium gluconate or sodium tartrate were prepared.

An aqueous stock solution was prepared containing the required amounts of sodium bicarbonate, tin chloride and sodium gluconate or sodium tartrate. The pH was adjusted to either 7.5 or 8-8.5 by dropwise addition of an aqueous solution of either hydrochloric acid (0.1 M) or sodium hydroxide (0.1 M). Aliquots of the stock solution were mixed with the required amount of (II-1-RGD), (II-1-PSMAt1), (II-2-PSMAt1), and (II-11-PSMAt1) (dissolved in a mixture of water/ethanol (70%/30%) to form the kit solutions outlined in Table 7, which were immediately frozen and lyophilised using a freeze dryer. The lyophilised kits were stored in a freezer prior to use.

Generator-produced ^{99m}TcO₄⁻ (200 MBq) in saline solution was then added to these kits, and the mixtures left to react at ambient temperature (around 22° C.) for 5 min, or heated at 100° C. for 5 min, prior to analysis by radio-iTLC and radio-HPLC.

Using a relatively “short” HPLC method (gradient mobile phase for 20 min; 1 ml min⁻¹ flow rate; linear increase from 100% A/0% B to 0% A/100% B; A=water containing 0.1% TFA, B=acetonitrile containing 0.1% TFA, analytical (4.6×150 mm, 5 μm) Agilent Zorbax Eclipse XDB-C18 column)), the species attributed as (Tc-III-1-PSMAt1) eluted at 11.0-12.5 min; (Tc-III-2-PSMAt1) eluted at 12.5-14.0 min.

Using a relatively “long” HPLC method (gradient mobile phase for 60 min; 1 ml min⁻¹ flow rate; 1% min⁻¹ linear increase from 100% A/0% B to 40% A/60% B; A=water containing 0.1% TFA, B=acetonitrile containing 0.1% TFA, analytical (4.6×150 mm, 5 μm) Agilent Zorbax Eclipse XDB-C18 column) to separate out cis and trans isomers, the species attributed as (Tc-III-1-PSMAt1) eluted at 38.89 and 39.25 min; (Tc-III-2-PSMAt1) eluted at 46.21 and 46.83 min.

Two separate iTLC analyses were undertaken, to enable quantification of ^{99m}Tc-colloids, unreacted ^{99m}TcO₄⁻ and (Tc-III-2-PSMAt1)/(Tc-III-2-PSMAt1).

To quantify amounts of unreacted ^{99m}TcO₄⁻, acetone was used as a mobile phase: R_{f} values: ^{99m}TcO₄⁻>0.9, ^{99m}Tc colloids<0.1 (Tc-III-1-PSMAt1)/(Tc-III-2-PSMAt1)<0.1.

To quantify ^{99m}Tc-colloid formation, a 1:1 mixture of methanol and 2M aqueous ammonium acetate solution was used as a mobile phase: ^{99m}TcO₄⁻>0.9, ^{99m}Tc colloids<0.1, (Tc-III-1-PSMAt1)/(Tc-III-2-PSMAt1)>0.9.

TABLE 7

| Kit | Components | Radiochemical Yield |
|---|---|---|
| 1 | (II-1-RGD): 1 mg (0.93 μmol); | (Tc-III-1-RGD) ≤ 34% |
| | Sodium gluconate (NaC₆H₁₁O₇): 1 mg (4.6 μmol); | |
| | SnCl₂•2H₂O: 50 μg (0.22 μmol); | |
| | NaHCO₃: 1.8 mg (21.4 μmol); | |
| | pH 8-8.5 | |
| | then added ^{99m}TcO₄⁻ in 150 μL saline/150 μL EtOH and | |
| | heated at 60° C. for 30 min. | |
| 2 | (II-1-RGD): 500 μg (0.47 μmol); | (Tc-III-1-RGD) 85% |
| | Sodium tartrate (Na₂C₄H₄O₆): 1.05 mg (4.6 μmol); | |
| | SnCl₂•2H₂O: 50 μg (0.22 μmol); | |
| | NaHCO₃: 1.8 mg (21.4 μmol); | |
| | pH 8-8.5 | |
| | then added ^{99m}TcO₄⁻ in 150 μL saline/150 μL EtOH and | |
| | heated at 60° C. for 30 min. | |
| 3 | (II-1-RGD): 125 μg (0.12 μmol); | (Tc-III-1-RGD) ≥ 90% |
| | Sodium tartrate: 0.26 mg (1.15 μmol); | |
| | SnCl₂•2H₂O: 25 μg (0.11 μmol); | |
| | NaHCO₃: 0.9 mg (10.7 μmol); | |
| | pH 8-8.5 | |
| | then added ^{99m}TcO₄⁻ in 250 μL saline/50 μL EtOH and | |
| | heated at 60° C. for 30 min. | |
| 4 | (II-1-RGD): 64 μg (0.06 μmol); | (Tc-III-1-RGD) 65% |
| | Sodium tartrate: 0.26 mg (1.15 μmol); | |
| | SnCl₂•2H₂O: 25 μg (0.11 μmol); | |
| | NaHCO₃: 0.9 mg (10.7 μmol); | |
| | pH 8-8.5 | |
| | then added ^{99m}TcO₄⁻ in 260 μL saline/40 μL EtOH and | |
| | heated at 60° C. for 30 min. | |
| 5 | (II-1-PSMAt1) 113 μg (0.11 μmol); | (Tc-III-1-PSMAt1) |
| | sodium tartrate: 0.26 mg (1.15 μmol); | 75.33 ± 3.0% |
| | SnCl₂•2H₂O: 25 μg (0.11 μmol); | at 22° C. |
| | NaHCO₃: 0.9 mg (10.71 μmol); | 81.2 ± 1.8% |
| | pH 8-8.5 | at 100° C. |
| | then added 200 MBq ^{99m}TcO₄⁻ in saline solution at | |
| | either 22° C. or 100° C. and incubated for 5 min. | |
| 6 | (II-2-PSMAt1) 119 μg (0.11 μmol); | (Tc-III-2-PSMAt1) |
| | sodium tartrate: 0.26 mg (1.15 μmol); | 83.53 ± 1.5% |
| | SnCl₂•2H₂O: 25 μg (0.11 μmol); | at 22° C. |
| | NaHCO₃: 0.9 mg (10.71 μmol); | 88.0 ± 0.6% |
| | pH 8-8.5 | at 100° C. |
| | then added 200 MBq ^{99m}TcO₄⁻ in saline solution at | |
| | either 22° C. or 100° C. and incubated for 5 min. | |
| 7 | (II-1-PSMAt1): 85 μg (0.08 μmol) | (Tc-III-1-PSMAt1) |
| | Sodium tartrate: 0.20 mg (0.87 μmol) | 81.1 ± 3.1% |
| | SnCl₂•2H₂O: 19.0 μg (0.08 μmol) | at 100° C. |
| | NaHCO₃: 0.68 mg (8.13 μmol) | |
| | pH 8.5 | |
| | then added 200 MBq ^{99m}TcO₄⁻ in saline solution at | |
| | 100° C. and incubated for 5 min | |
| 8 | (II-1-PSMAt1): 85 μg (0.08 μmol) | (Tc-III-1-PSMAt1) |
| | Sodium tartrate: 0.20 mg (0.87 μmol) | 83.5 ± 5.8% |
| | SnCl₂•2H₂O: 19.0 μg (0.08 μmol) | at 100° C. |
| | NaHCO₃: 0.68 mg (8.13 μmol) | |
| | pH 7.5 | |
| | then added 200 MBq ^{99m}TcO₄⁻ in saline solution at | |
| | 100° C. and incubated for 5 min | |
| 9 | (II-1-PSMAt1): 85 μg (0.08 μmol) | (Tc-III-1-PSMAt1) |
| | Sodium tartrate: 0.26 mg (1.15 μmol) | 89.9 ± 3.8% |
| | SnCl₂•2H₂O: 25.0 μg (0.11 μmol) | at 100° C. |
| | NaHCO₃: 0.90 mg (10.71 μmol) | |
| | pH 7.5 | |
| | then added 200 MBq ^{99m}TcO₄⁻ in saline solution at | |
| | 100° C. and incubated for 5 min | |
| 10 | (II-1-PSMAt1): 85 μg (0.08 μmol) | (Tc-III-1-PSMAt1) |
| | Sodium tartrate: 0.26 mg (1.15 μmol) | 94.0 ± 2.7% |
| | SnCl₂•2H₂O: 19.0 μg (0.08 μmol) | at 100° C. |
| | NaHCO₃: 0.90 mg (10.71 μmol) | |
| | pH 7.5 | |
| | then added 200 MBq ^{99m}TcO₄⁻ in saline solution at | |
| | 100° C. and incubated for 5 min | |
| 11 | (II-1-PSMAt1): 85 μg (0.08 μmol) | (Tc-III-1-PSMAt1) |
| | Sodium tartrate: 0.53 mg (2.29 μmol) | 98.0 ± 0.5% |
| | SnCl₂•2H₂O: 19.0 μg (0.08 μmol) | at 100° C. |
| | NaHCO₃: 0.90 mg (10.71 μmol) | |
| | pH 7.5 | |
| | then added 200 MBq ^{99m}TcO₄⁻ in saline solution at | |
| | 100° C. and incubated for 5 min | |
| 12 | (II-1-PSMAt1): 85 μg (0.08 μmol) | (Tc-III-1-PSMAt1) |
| | Sodium tartrate: 0.53 mg (2.29 μmol) | 97.0 ± 1.0% |
| | SnCl₂•2H₂O: 19.0 μg (0.08 μmol) | at 100° C. |
| | NaHCO₃: 0.90 mg (10.71 μmol) | |
| | pH 8.5 | |
| | then added 200 MBq ^{99m}TcO₄⁻ in saline solution at | |
| | 100° C. and incubated for 5 min | |
| 13 | (II-11-PSMAt1) 119 μg (0.11 μmol); | (Tc-III-11-PSMAt1) |
| | sodium tartrate: 0.26 mg (1.15 μmol); | 90% at 100° C. |
| | SnCl₂•2H₂O: 25 μg (0.11 μmol); | |
| | NaHCO₃: 0.9 mg (10.71 μmol); | |
| | pH 8-8.5 | |
| | then added 200 MBq ^{99m}TcO₄⁻ in saline solution at | |
| | 100° C. and incubated for 5 min. | |

The amounts of tin(II) chloride, sodium bicarbonate and sodium gluconate reagents used in Kit 1 replicate those in the tetrofosmin kit. Addition of generator-produced ^{99m}TcO—in saline solution (20-55 MBq) to the contents of Kit 1, followed by heating at 60° C. for 30 min, resulted in formation of Compound (Tc-III-1-RGD) in radiochemical yields of up to 34%. Replacing sodium gluconate with sodium tartrate in the kit mixture whilst lowering the amount of Compound (II-1-RGD) conjugate from 1 mg to 0.5 mg, increased radiochemical yields to 85% (Kit 2).

In Kit 3, radiochemical yields of ≥90% were consistently achieved (93.0±1.0%, n=4), with 45-65 MBq of ^{99m}TcO—and only 125 μg of Compound (II-1-RGD). In Kit 3, sodium tartrate and tin(II) chloride amounts were also reduced. However, further decreasing Compound (II-1-RGD), to 63 jig in Kit 4, reduced radiochemical yields to 65%. All radiolabelling reactions were undertaken in a mixture of saline and ethanol to dissolve Compound (II-1-RGD); lower amounts of ethanol were required for kits containing lower amounts of Compound (II-1-RGD).

In Kit 5 and Kit 6, it is shown that the substitution of the aryl phosphine substituent with an electron donating group, in this case a phenyl substituted in the para position with a methyl group, improves the yield at both room temperature and 100° C. In Kit 13, it is shown that a more electron-donating group, in this case a phenyl substituted in the para position with a methoxy group, improves the yield at 100° C. even more than a methyl substituent using the same method.

### Example 15a—Kit Radiolabelling of (II-1-PSMAt1), (II-2-PSMAt1), and (II-11-PSMAt1) with ¹⁸⁸ReO₄⁻

A sample of ¹⁸⁸ReO₄⁻ in saline solution were obtained from an Oncobeta ¹⁸⁸W/¹⁸⁸Re generator. ¹⁸⁸ReO₄⁻ was “pre-concentrated”: a solution of ¹⁸⁸ReO₄⁻ in saline was passed through a Ag⁺ cartridge (Dionex OnGuard™ II Ag; preconditioned with 10 mL water) and onto a QMA cartridge (Sep-Pak® Light (46 mg) Accell™ Plus QMA Carbonate; preconditioned with 5 mL EtOH, then 10 mL water), where the ¹⁸⁸ReO₄⁻ was trapped. The QMA cartridge was then washed with water (4 mL), before eluting the ¹⁸⁸ReO₄⁻ in a small volume of saline (0.9% NaCl in water, w/v). This “pre-concentration” process could be combined with the generator-elution, facilitating direct concentration of the generator eluate while minimising radioactivity handling. Direct concentration of the eluate was achieved using tubing to connect the generator outlet to the two cartridges (in tandem), which was in turn attached to a vacuum pump via two or more receiver vials.

Aqueous saline solution containing ¹⁸⁸ReO₄⁻ (125 μL, 30-450 MBq) was added to an aqueous solution of sodium citrate (1 M, 50 μL) and stannous chloride (3.75 mg), and heated at 90° C. for 30 min. An aliquot of this solution (50 μL, 10-150 MBq) was then added to the contents of either two (II-1-PSMAt1) kits, two (II-2-PSMAt1) kits, or two (II-11-PSMAt1) kits (as described in Table 5), to give a solution of pH 8-8.5, which was then heated at 90° C. for 30 min. Aliquots of the reaction solution were then analysed by reverse phase C18 radio-HPLC (30 min method).

Aliquots of the reaction solution were then analysed by reverse phase C18 radio-HPLC.

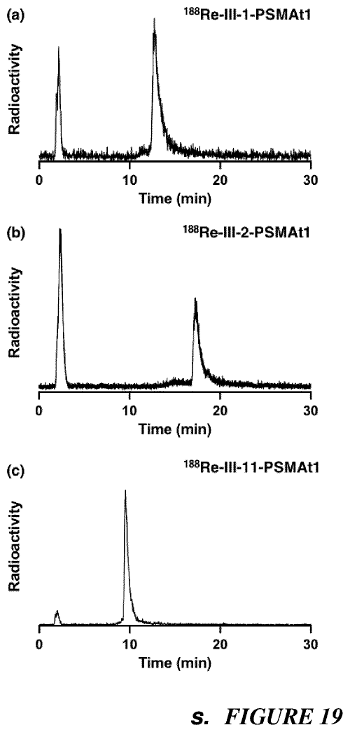

Unreacted ¹⁸⁸ReO₄⁻ and ¹⁸⁸Re-citrate eluted at 2.0-2.3 min. The species attributed as (Re-III-1-PSMAt1) eluted at 12.7 min in 73% radiochemical yield (FIG. 19a). (Re-III-2-PSMAt1) eluted at 17.5 min in 46% radiochemical yield (FIG. 19b). (Re-III-11-PSMAt1) eluted at 9.53 mins in 90% radiochemical yield (FIG. 19c).

### Example 15b—Purification and Stability of (Re-III-1-PSMAt1) and (Re-III-2-PSMAt1)

Crude reaction mixture containing either (Re-II-1-PSMAt1) or (Re-II-2-PSMAt1), prepared as described above, were applied to a reverse phase C18 analytical HPLC column and isolated using the following linear HPLC gradient: 0 min, 100% A/0% B to 60 min, 40% A/60% B, 1 mL min⁻¹ flow rate. Fractions containing either [(Re-II-1-PSMAt1) (eluted at 38-40 min as a double peak) or (Re-II-2-PSMAt1) (eluted at 46-48 min as a double peak) were immediately frozen and lyophilized. The resulting samples of (Re-II-1-PSMAt1) or (Re-II-2-PSMAt1) were dissolved in phosphate buffered saline and measured 95% radiochemical purity (by analytical C18 radio-HPLC and radio-iTLC).

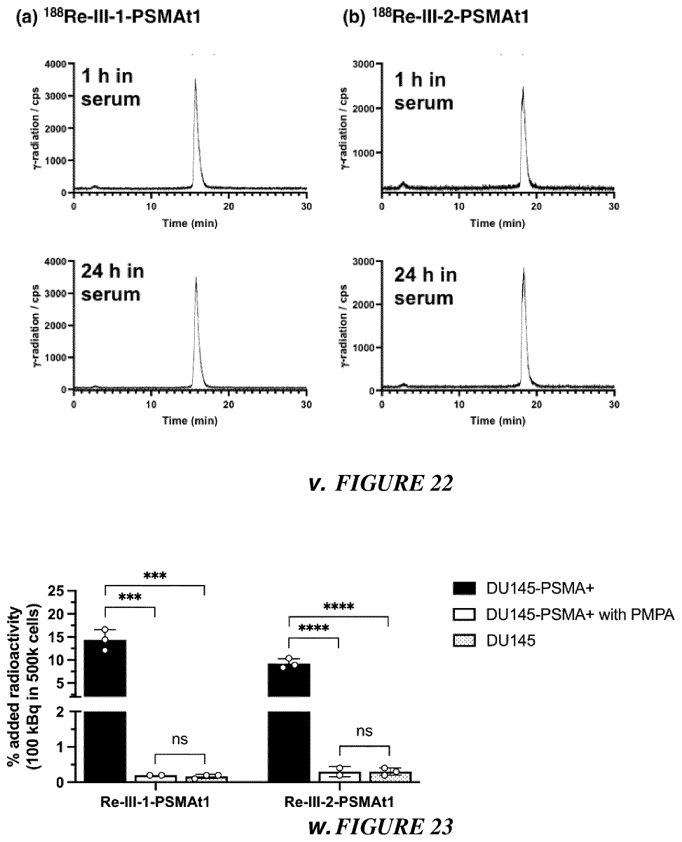

Solutions of (Re-II-1-PSMAt1) or (Re-II-2-PSMAt1) in phosphate buffered saline (20 μL, 0.5-1.5 MBq) were added to samples of human serum (180 μL) and incubated at 37° C. At 1 and 24 h, samples were treated with ice-cold acetonitrile (300 μL) to precipitate and remove serum proteins. Acetonitrile in the supernatant was then removed by evaporation under a stream of N₂ gas. The final solution was then analysed by reverse-phase analytical radioHPLC (FIGS. 22a and 22b).

### Example 16—Uptake of (Re-III-1-PSMAt1) and (Re-III-2-PSMAt1) in Prostate Cancer Cell Lines

A panel of cell lines were selected that either expressed GCP(II)/PSMA-(DU145-PSMA (genetically modified to express PSMA) [1], or had low GCP(II)/PSMA expression (DU145 (HTB-81)). The cell lines were cultured in RPMI 1640 medium (R₀₈₈₃, Sigma) containing 10% foetal bovine serum, 2 mM L-glutamine, and 100 U·mL⁻¹ penicillin and 100 μg·mL⁻¹ streptomycin. Cells were maintained at 37° C. and 5% CO₂. Cells were seeded in 6-well plates at a density of 5×10⁵ cells per well in 2 mL complete media to achieve 70-80% confluency the following day. The cell medium (1 mL/well) was replaced 1 h prior to treating the cells. Solutions containing either (Re-III-1-PSMAt1) or (Re-III-2-PSMAt1) (50 kBq, in 5-10 uL of phosphate buffered saline, >95% radiochemical purity) were added to each well, and the cells incubated at 37° C. for 1 h. Uptake studies were also performed after a 2 min incubation with the PSMA inhibitor 2-(phosphonomethyl)pentane-1,5-dioic acid (PMPA; 30 μL of 750 μM PMPA solution/well). After 1 h incubation, the supernatant was removed and the cells were washed with cold phosphate buffered saline solution (3×1 mL). The cells were lysed with cold radioimmunoprecipitation assay buffer (RIPA buffer, 500 μL; 150 mM sodium chloride, 0.1% w/w sodium dodecyl sulfate (SDS), 0.5% w/w sodium deoxycholate (NaDOC), 1% w/w Triton-X) and samples were collected for radioactivity counting. Results are depicted as means±SD of independent biological experiments (performed on different days with different radiotracer preparations).

(Re-III-1-PSMAt1) and (Re-III-2-PSMAt1) exhibited uptake in DU145-PSMA+ cells (14.37±2.25% AR [percentage added radioactivity], and 9.23±1.04% AR respectively). This uptake was specific: DU145-PSMA+ cell uptake of (Re-III-1-PSMAt1) and (Re-III-2-PSMAt) could be blocked with PMPA, and there was negligible uptake in parental DU145 cells (FIG. 23).

### Example 17—Preparation of (¹⁸⁶Re-III-1-PSMAt) and Kit Radiolabeling

¹⁸⁶Re-III-1-PSMA was prepared in two steps from a saline solution containing ¹⁸⁶ReO₄⁻.

SnCl₂·2H₂O (15 mg) was dissolved in aqueous sodium citrate solution (100 μL, 1 M). A sample of this solution (25 μL) was added to an aqueous saline solution containing ¹⁸⁶ReO₄⁻ (10 MBq, 65 μL). The reaction mixture was heated to 90° C. for 30 min, yielding ¹⁸⁶Re(V)-citrate in 96% radiochemical yield.

Following this, ¹⁸⁶Re(V)-citrate (4-5 MBq, 50 μL) was added to a pre-fabricated, lyophilized kit (Table 8, also used for ¹⁸⁸Re radiolabelling), containing sodium carbonate, sodium tartrate, tin chloride and DP^{Ph}-PSMAt. This solution was heated at 90° C. for 30 min, resulting in formation of ¹⁸⁶Re-DP1-PSMA in 5.5% radiochemical yield as determined by radio-HPLC.

TABLE 8
Lyophilised kit formulations for (III-
1-PSMAt) for ¹⁸⁶Re radiolabelling.

| | moles/ | weight/ |
|---|---|---|
| Components: | μmol | mg |
| (II-1-PSMAt1) | 0.22 | 0.22 |
| SnCl₂•2H₂O | 0.22 | 0.05 |
| Sodium tartrate | 2.29 | 0.53 |
| NaHCO₃ | 21.42 | 1.80 |

Solutions of (¹⁸⁶Re-III-1-PSMAt1) prepared from kits as described above were applied to a reverse phase C18 analytical HPLC column and isolated using the following linear HPLC gradient: 0 min, 100% A/0% B to 60 min, 40% A/60% B, 1 mL min⁻¹ flow rate (A=water containing 0.1% TFA, B=acetonitrile containing 0.1% TFA). Fractions containing (¹⁸⁶Re-III-1-PSMAt1) eluted at 39.8 mins, and were immediately frozen and lyophilised. Analytical reverse-phase HPLC indicated that radiochemical purity of (¹⁸⁶Re-III-1-PSMAt1) was >95%. This radiolabeled species co-eluted with the non-radioactive (^{nat}Re-III-1-PSMAt1) standard.

### Example 18—Uptake of (¹⁸⁶Re-III-1-PSMAt1) in Prostate Cancer Cell Lines

GCP(II)/PSMA-expressing cells, DU145-PSMA+ and LNCaP cells, were suspended in RPMI media (5 million cells, 1 mL). (¹⁸⁶Re-III-1-PSMAt1) (10,000 cpm, in-10 μL of phosphate buffered saline, >95% radiochemical purity) was added to each cell sample, and the cells incubated at 37° C. for 1 h, with constant agitation. Additionally, non-specific uptake was also determined by using non-GCP(II)/PSMA-expressing cells (DU145) or by blocking PSMA-expressing cells (DU145-PSMA+ and LNCaP cells) with the PSMA-inhibitor, PMPA (30 μL of a 750 uM PMPA solution/5 million cells). After 60 min incubation, the supernatant was removed and the cells were washed three times with ice cold phosphate buffered saline solution. The cells were treated with ice cold RIPA buffer (500 μL, 150 mM sodium chloride, 0.1% w/w sodium dodecyl sulfate (SDS), 0.5% w/w sodium deoxycholate (NaDOC), 1% w/w Triton-X) to lyse the cells, and samples collected for radioactivity counting. Uptake of ¹⁸⁶Re-DP1-PSMA measured 4.23±0.99% AR [percentage added radioactivity], in DU145-PSMA+ cells, and this decreased to 0.08±0.14% AR in PSMA-negative DU145 cells, and 0.21±0.16% AR when co-incubated with an excess of PMPA. Uptake of ¹⁸⁶Re-DP1-PSMA measured 3.98±0.98% AR in LNCaP cells, and this decreased to 0.55±0.15% AR when co-incubated with an excess of PMPA (see FIG. 24).

### Example 19—Biodistributions of (⁸⁸Re-III-1-PSMAt1) and (⁸⁸Re-III-2-PSMAt1) in Mice Bearing Prostate Cancer Tumours

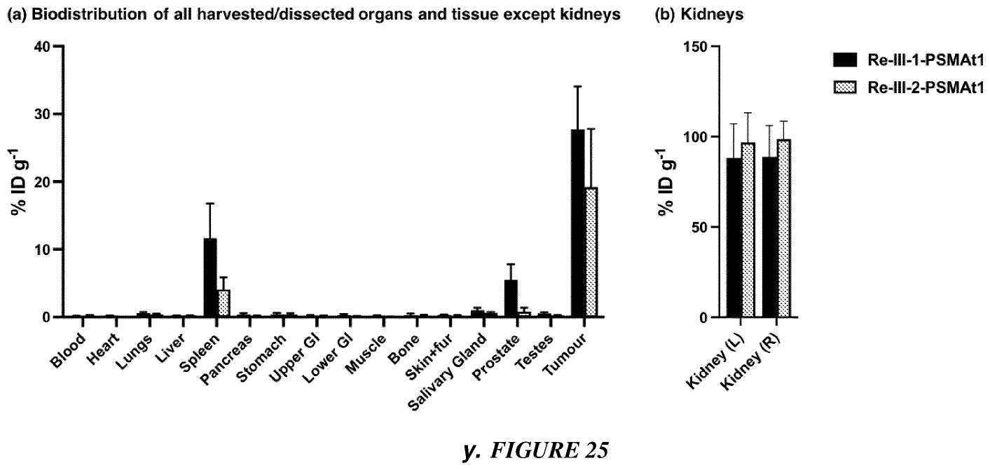

The biodistributions of (¹⁸⁸Re-III-1-PSMAt1) and (¹⁸⁸Re-III-2-PSMAt1) were assessed in SCID/Beige mice bearing DU145-PSMA+ tumours (FIG. 25). Each animal was administered either (¹⁸⁸Re-III-1-PSMAt1) or (¹⁸⁸Re-III-2-PSMAt1), and euthanized at 2 h post-injection, followed by organ harvesting for ex vivo radioactivity counting. For animals administered (¹⁸⁸Re-III-1-PSMAt1), a radioactivity concentration of 27.7±6.4% ID g⁻¹ (percentage injected dose per gram) was measured in tumours 2 h post-injection. For animals administered (¹⁸⁸Re-III-2-PSMAt1), a radioactivity concentration of 19.2±8.6% ID g⁻¹ was measured in tumours 2 h post-injection. Both compounds cleared circulation via a renal pathway, as evidenced by high concentrations of radioactivity measured in the kidneys. Biodistribution data also showed that both compounds had low retention in non-target, healthy organs/tissue, except for organs known to express PSMA (spleen and prostate). In this experiment, there were no observed statistically significant differences between the biodistribution profiles of (¹⁸⁸Re-III-1-PSMAt1) compared to (¹⁸⁸Re-III-2-PSMAt1) at 2 h post-injection.

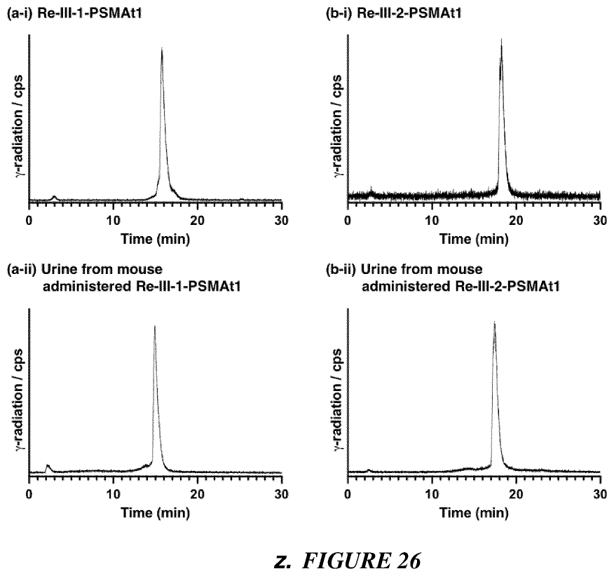

Urine was collected from mice administered either (¹⁸⁸Re-III-1-PSMAt1) or (¹⁸⁸Re-III-2-PSMAt1) at 2 h post-injection, and analysed by reverse-phase radio-HPLC. Radio-chromatograms showed that both (¹⁸⁸Re-III-1-PSMAt1) and (¹⁸⁸Re-III-2-PSMAt1) are highly stable, with >94% of radioactivity associated with either (¹⁸⁸Re-III-1-PSMAt1) or (¹⁸⁸Re-III-2-PSMAt1) respectively. (FIG. 26).

The GCP(II)/PSMA-expressing cell line used in these experiments was a genetically modified daughter cell line of DU145, DU145-PSMA+. This cell line had previously been transduced to express full-length human GCP(II)/PSMA, following F. Kampmeier, J. D. Williams, J. Maher, G. E. Mullen and P. J. Blower, EJNMMI Res., 2014. 4, 13. These cells were cultured in DMEM medium supplemented with 10% foetal bovine serum, 2 mM L-glutamine, and penicillin/streptomycin. To prepare for experiments, cells were grown at 37° C. in an incubator with humidified air equilibrated with 5% CO₂.

Animal studies complied with the guidelines on responsibility in the use of animals in bioscience research of the U.K. Research Councils and Medical Research Charities, under U.K. Home Office project and personal licenses. Subcutaneous prostate cancer xenografts were produced in SCID/beige mice (male, 7-12 weeks old) by injecting 4×10⁶ DU145-PSMA or DU145 cells suspended in PBS (100 μL) on the right shoulder. Biodistribution studies were performed once a tumour had reached 5-10 mm in diameter (3-4 weeks after injection). For imaging purposes, the mice were anaesthetised, positioned on the scanner, and tail vein cannulated. For biodistribution, the mice were anaesthetised, the radiotracers were injected via the tail vein.

1-26. (canceled)

27. A conjugated diphosphine precursor compound according to Formula (II) that is suitable for preparing a conjugated radiolabelled agent: 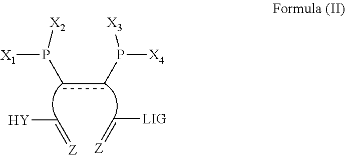
wherein;
each Z is independently O or S;
Y is NH or O;
X₁, X₂, X₃ and X₄ are each independently a substituted or unsubstituted C₅-C₈aryl group, a substituted or unsubstituted 5- to 8-membered heteroaryl group or a substituted or unsubstituted C₃-C₈ cycloalkyl group wherein each substituent is selected from the group consisting of a C₁-C₄alkyl group, C₅-C₁₂aryl or heteroaryl group, a C₁-C₄ acylamido group, a sulfylhydro group, a C₁-C₄ alkylthio group, a C₁-C₄(di)alkylphosphino group, a hydroxy group, a C₁-C₄alkoxy group, a carboxyl group, a C₁-C₄(di)alkylamino group and a C₁-C₄alkoxy-(CH₂CH₂O)ₙ group wherein n is 1, 2, 3, 4, 5, 6, 7, 8, 9 or 10.
the shortest linear chain of carbon atoms between the two Z groups is 4 to 7;
LIG comprises a ligand with a binding motif corresponding to a biological target; and
the compound is not

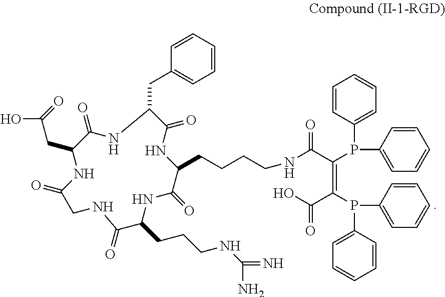

28. A conjugated diphosphine precursor compound of claim 27, according to Formula (IIa) that is suitable for preparing a conjugated radiolabelled agent: 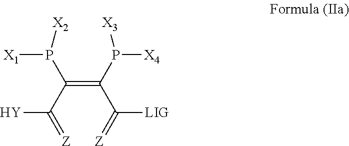
wherein;
each Z is independently O or S;
Y is NH or O;
X₁, X₂, X₃ and X₄ are each independently a substituted or unsubstituted C₅-C₈aryl group wherein each substituent is selected from the group consisting of a C₁-C₄alkyl group, a C₁-C₄alkoxy group, a C₁-C₄(di)alkylamino group and a C₁-C₄alkoxy-(CH₂CH₂O)ₙ group wherein n is 1, 2, 3, 4, 5, 6, 7, 8, 9 or 10; and
LIG comprises a prostate specific membrane antigen targeting ligand (PSMAt), cyclic(Arg-Gly-Asp-dPhe-Lys) (RGD), pentixafor peptide, a minigastrin peptide analogue for targeting cholecystokinin-2 receptor, a c-Met-targeting peptide, an alpha-MSH peptide, a bisphosphonate, a folate or a carbohydrate.

29. The conjugated diphosphine precursor compound of claim 28, wherein the conjugated diphosphine precursor is according to Formula (IIb) and/or Formula (IIc): 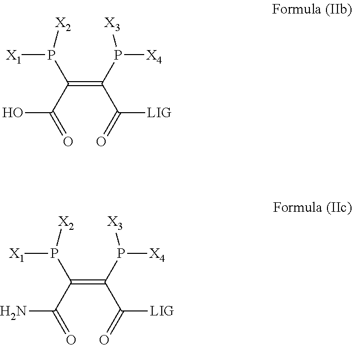
wherein;
X₁, X₂, X₃ and X₄ are each independently p-tolyl, m-tolyl, o-tolyl, 2,3-xylyl, 2,4-xylyl, 2,5-xylyl, 2,6-xylyl, 3,4-xylyl, 3,5-xylyl,r p-methoxyphenyl, o-methoxyphenyl, 4-(MeO(CH₂CH₂O))phenyl, 4-(MeO(CH₂CH₂O)₂)phenyl, 4-(MeO(CH₂CH₂O)₃)phenyl or 4-dimethylaminophenyl; and
LIG comprises a prostate specific membrane antigen targeting ligand (PSMAt) or cyclic(Arg-Gly-Asp-dPhe-Lys) (RGD).

30. A compound of claim 27, that is:

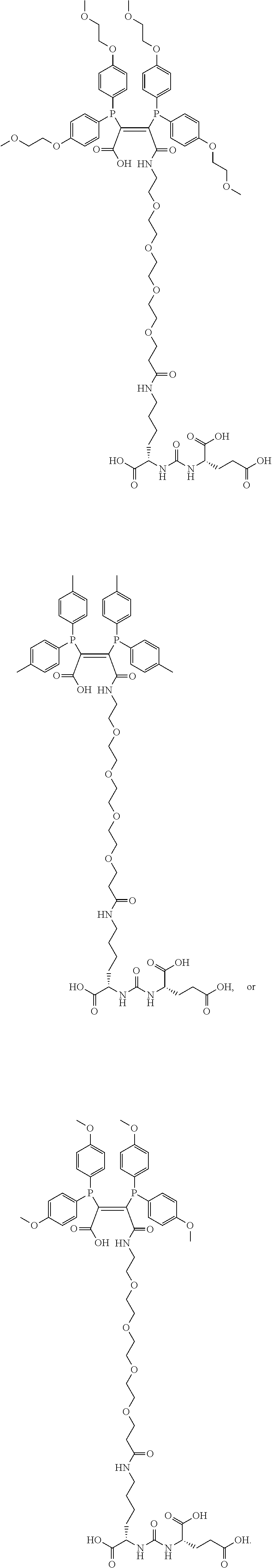 [SMILES: COCCOc1ccc(P(/C(C(=O)O)=C(/C(=O)NCCOCCOCCOCCOCCC(=O)NCCCC[C@H](NC(=O)N[C@@H](CCC(=O)O)C(=O)O)C(=O)O)P(c2ccc(OCCOC)cc2)c2ccc(OCCOC)cc2)c2ccc(OCCOC)cc2)cc1 ; Cc1ccc(P(/C(C(=O)O)=C(/C(=O)NCCOCCOCCOCCOCCC(=O)NCCCC[C@H](NC(=O)N[C@@H](CCC(=O)O)C(=O)O)C(=O)O)P(c2ccc(C)cc2)c2ccc(C)cc2)c2ccc(C)cc2)cc1 ; COc1ccc(P(/C(C(=O)O)=C(/C(=O)NCCOCCOCCOCCOCCC(=O)NCCCC[C@H](NC(=O)N[C@@H](CCC(=O)O)C(=O)O)C(=O)O)P(c2ccc(OC)cc2)c2ccc(OC)cc2)c2ccc(OC)cc2)cc1]

31. A diphosphine precursor compound according to Formula (I) that is suitable for preparing a conjugated radiolabelled agent: 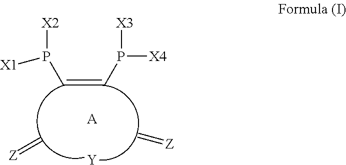
wherein
ring A is a 5, 6, 7 or 8 membered ring;
each Z is independently O or S;
Y is NH or O;
X₁, X₂, X₃ and X₄ are each independently a substituted or unsubstituted C₅-C₈aryl group, a substituted or unsubstituted 5- to 8-membered heteroaryl group wherein any substituents selected from the group consisting of a C₁-C₄alkyl group, C₅-C₁₂aryl or heteroaryl group, a C₁-C₄ acylamido group, a sulfylhydro group, a C₁-C₄ alkylthio group, a C₁-C₄(di)alkylphosphino group, a hydroxy group, a C₁-C₄alkoxy group, a carboxyl group, a C₁-C₄(di)alkylamino group and a C₁-C₄alkoxy-(CH₂CH₂O)ₙ group wherein n is 1, 2, 3, 4, 5, 6, 7, 8, 9 or 10; and
the compound is not

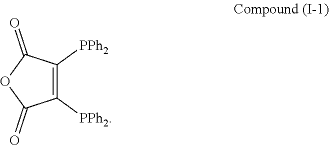

32. A diphosphine precursor compound of claim 30, according to Formula (Ia) that is suitable for preparing a conjugated radiolabelled agent: 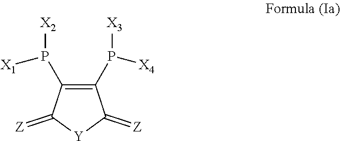
wherein
each Z is independently O or S;
Y is NH or O;
X₁, X₂, X₃ and X₄ are each independently a substituted C₅-C₈aryl group having one or more substituents selected from the group consisting of a C₁-C₄alkyl group, a C₁-C₄alkoxy group, a C₁-C₄(di)alkylamino group and a C₁-C₄alkoxy-(CH₂CH₂O)ₙ group wherein n is 1, 2, 3, 4, 5, 6, 7, 8, 9 or 10.

33. The diphosphine precursor compound of claim 32 wherein the diphosphine precursor is according to Formula (Ib) and/or (Ic): 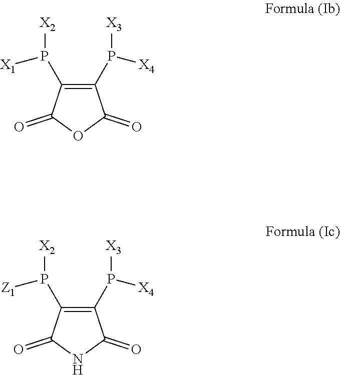
wherein;
X₁, X₂, X₃ and X₄ are each independently p-tolyl, m-tolyl, o-tolyl, 2,3-xylyl, 2,4-xylyl, 2,5-xylyl, 2,6-xylyl, 3,4-xylyl, 3,5-xylyl, p-methoxyphenyl, o-methoxyphenyl, 4-(MeO(CH₂CH₂O))phenyl, 4-(MeO(CH₂CH₂O)₂)phenyl, 4-(MeO(CH₂CH₂O)₃)phenyl or 4-dimethylaminophenyl.

34. A compound of claim 27 wherein X₁, X₂, X₃ and X₄ are the same.

35. A compound of claim 31 wherein X₁, X₂, X₃ and X₄ are the same.

36. A radiolabelled diphosphine complex comprising at least two compounds of claim 27 as ligands that are co-ordinated with one or more radionuclides selected from ^{99m}Tc, ²¹²Pb, ²¹²Bi ²¹³Bi, ¹⁸⁶Re, ¹⁸⁸Re, ⁸⁹Zr, ⁶⁷Ga, ⁶⁸Ga, ⁶⁷Cu, ⁶⁴Cu, ⁶²Cu, ⁶¹Cu, ⁶⁰Cu, ⁶²Zn and ⁵²Mn; and
the complex is not; 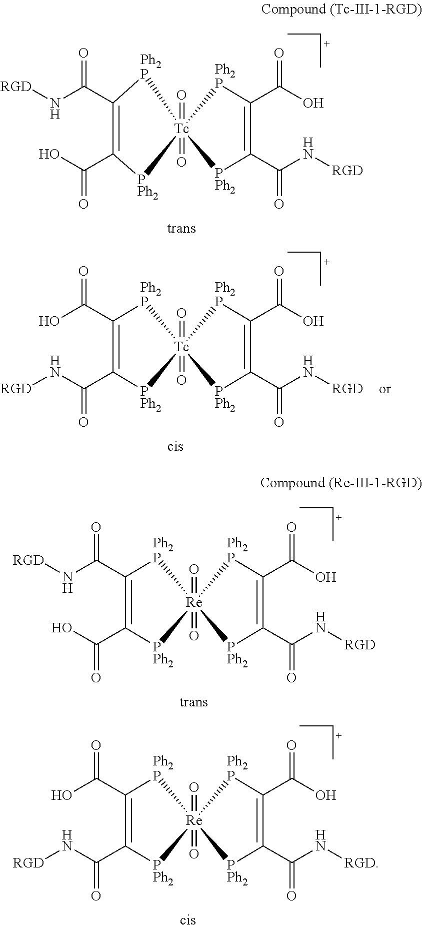
wherein in compound (Tc-III-1-RGD) Tc is ^{99m}Tc, and in compound (Re-III-1-RGD) Re is selected from ¹⁸⁶Re and ¹⁸⁸Re.

37. A radiolabelled conjugated diphosphine complex of claim 36 that is either:
(a) according to Formula (M-IIIa-trans) or Formula (M-IIIa-cis) or a mixture thereof 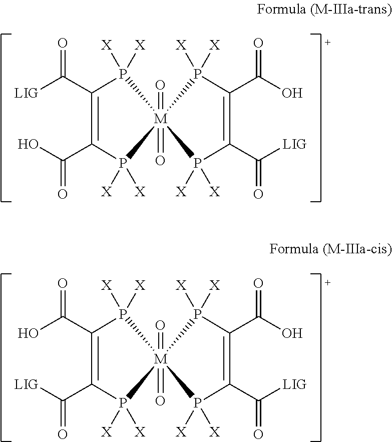
wherein M is a radionuclide selected from one or more of ^{99m}Tc, ¹⁸⁶Re and ¹⁸⁸Re; or
(b) according to Formula (M-IIIb-trans) or Formula (M-IIIb-cis) or a mixture thereof 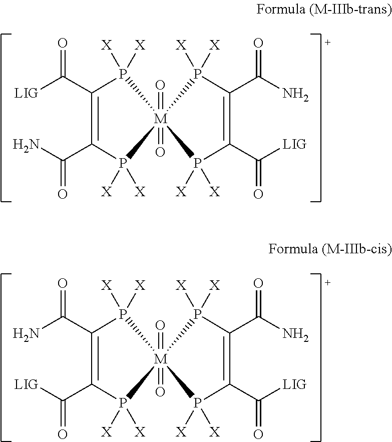
wherein M is a radionuclide selected from one or more of ^{99m}Tc, ¹⁸⁶Re and ¹⁸⁸Re; or
(c) according to Formula (Cu-IIIc-A) or Formula (Cu-IIIc-B) or a mixture thereof; 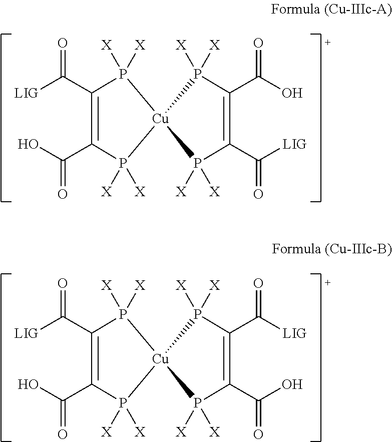
wherein Cu is selected from ⁶⁷Cu, ⁶⁴Cu, ⁶²Cu, ⁶¹Cu and ⁶⁰Cu; or
(d) according to Formula (Cu-IIId-A) or Formula (Cu-IIId-B) or a mixture thereof; 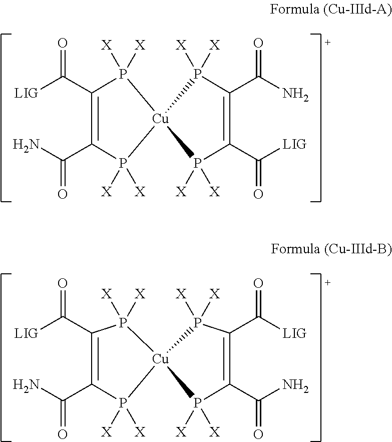
wherein Cu is selected from ⁶⁷Cu, ⁶⁴Cu, ⁶²Cu, ⁶¹Cu and ⁶⁰Cu;
and wherein;
X is a phenyl group having one or more substituents selected from the group consisting of a C₁-C₄alkyl group and a C₁-C₄alkoxy group; and
LIG comprises a prostate specific membrane antigen targeting ligand (PSMAt), cyclic(Arg-Gly-Asp-dPhe-Lys) (RGD), pentixafor peptide, a minigastrin peptide analogue for targeting cholecystokinin-2 receptor, a c-Met-targeting peptide, an alpha-MSH peptide, a bisphosphonate, a folate or a carbohydrate
optionally, wherein the radiolabelled conjugated diphosphine complex is either:
(a) according to Formula (M-IIIa-trans) or Formula (M-IIIa-cis) or a mixture thereof 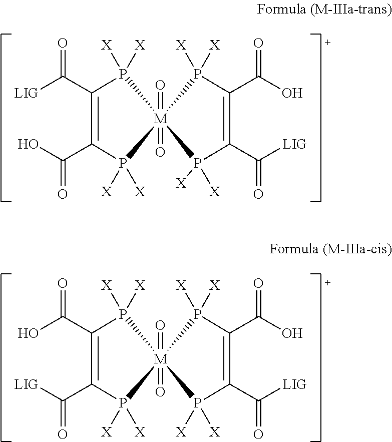
wherein M is a radionuclide selected from one or more of ^{99m}Tc, ¹⁸⁶Re and ¹⁸⁸Re; or
(b) according to Formula (M-IIIb-trans) or Formula (M-IIIb-cis) or a mixture thereof 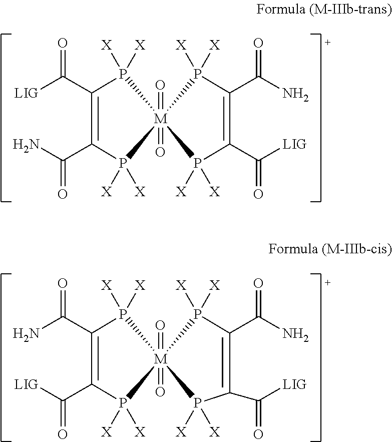
wherein M is a radionuclide selected from one or more of ^{99m}Tc, ¹⁸⁶Re and ¹⁸⁸Re;
and wherein;
X is a phenyl group having one or more substituents selected from the group consisting of a C₁-C₄alkyl group and a C₁-C₄alkoxy group; and
LIG comprises a prostate specific membrane antigen targeting ligand (PSMAt), cyclic(Arg-Gly-Asp-dPhe-Lys) (RGD), pentixafor peptide, a minigastrin peptide analogue for targeting cholecystokinin-2 receptor, a c-Met-targeting peptide, an alpha-MSH peptide, a bisphosphonate, a folate or a carbohydrate;
optionally, wherein;
X is p-tolyl, m-tolyl, o-tolyl, 2,3-xylyl, 2,4-xylyl, 2,5-xylyl, 2,6-xylyl, 3,4-xylyl, 3,5-xylyl, p-methoxyphenyl, o-methoxyphenyl, 4-(MeO(CH₂CH₂O))phenyl, 4-(MeO(CH₂CH₂O)₂)phenyl, 4-(MeO(CH₂CH₂O)₃)phenyl or 4-dimethylaminophenyl; and
LIG comprises a prostate specific membrane antigen targeting ligand (PSMAt) or cyclic(Arg-Gly-Asp-dPhe-Lys) (RGD).

38. A complex according to claim 36 according to Formula (M-IIIa-trans) or Formula (M-IIIa-cis) or a mixture thereof;
optionally, wherein the complex is an approximate 1:1 mixture of the cis/trans isomers.

39. A method of making a conjugated diphosphine precursor compound of claim 27, or Compound (II-1-RGD), comprising a step of mixing a diphosphine precursor compound or Compound (I-1) and LIG-H in the presence of a base, wherein LIG comprises a peptide or carbohydrate ligand with a binding motif corresponding to a biological target;
optionally, wherein the base is N,N-diisopropylethylamine which is added dropwise and the reaction is conducted in N,N-dimethylformamide at room temperature;
wherein the diphosphine precursor compound has a structure according to Formula (I): 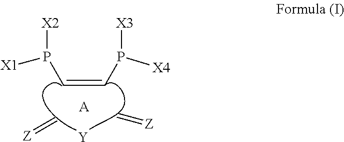
where
ring A is a 5, 6, 7 or 8 membered ring;
each Z is independently O or S;
Y is NH or O;
X₁, X₂, X₃ and X₄ are each independently a substituted or unsubstituted C₅-C₈aryl group, a substituted or unsubstituted 5- to 8-membered heteroaryl group wherein any substituents selected from the group consisting of a C₁-C₄alkyl group, C₅-C₁₂aryl or heteroaryl group, a C₁-C₄ acylamido group, a sulfylhydro group, a C₁-C₄ alkylthio group, a C₁-C₄(di)alkylphosphino group, a hydroxy group, a C₁-C₄alkoxy group, a carboxyl group, a C₁-C₄(di)alkylamino group and a C₁-C₄alkoxy-(CH₂CH₂O)ₙ group wherein n is 1, 2, 3, 4, 5, 6, 7, 8, 9 or 10; and
the compound is not

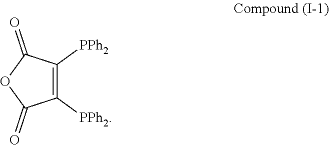

40. A method of making the radiolabelled conjugated diphosphine complex of claim 36, Compound (Tc-III-1-RGD), Compound (¹⁸⁶Re-III-1-RGD) or Compound (Re-III-1-RGD), comprising the step of mixing a conjugated diphosphine precursor compound or Compound (II-1-RGD) with a radionuclide, in the presence of an intermediate ligand, a reducing agent, a buffer and a solvent
optionally, wherein the radionuclide is selected from one of more of ^{99m}Tc, ²¹²Bi, ²¹³Bi, ¹⁸⁶Re, ¹⁸⁸Re, ⁸⁹Zr, ⁶⁷Ga, ⁶⁸Ga, ⁶⁷Cu, ⁶⁴Cu, ⁶²Cu, ⁶¹Cu, ⁶⁰Cu and ⁵²Mn, the intermediate ligand is sodium tartrate, the reducing agent is tin(II) chloride, the buffer sodium hydrogen carbonate and the solvent is preferably selected from one or more of water, a saline solution, methanol, ethanol, propanol and isopropanol;
wherein the conjugated diphosphine precursor compound has a structure according to Formula (II): 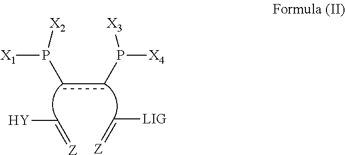
wherein;
each Z is independently O or S;
Y is NH or O;
X₁, X₂, X₃ and X₄ are each independently a substituted or unsubstituted C₅-C₈ aryl group, a substituted or unsubstituted 5- to 8-membered heteroaryl group or a substituted or unsubstituted C₃-C₈ cycloalkyl group wherein each substituent is selected from the group consisting of a C₁-C₄alkyl group, C₅-C₁₂aryl or heteroaryl group, a C₁-C₄ acylamido group, a sulfylhydro group, a C₁-C₄ alkylthio group, a C₁-C₄(di)alkylphosphino group, a hydroxy group, a C₁-C₄alkoxy group, a carboxyl group, a C₁-C₄(di)alkylamino group and a C₁-C₄alkoxy-(CH₂CH₂O)ₙ group wherein n is 1, 2, 3, 4, 5, 6, 7, 8, 9 or 10.
the shortest linear chain of carbon atoms between the two Z groups is 4 to 7;
LIG comprises a ligand with a binding motif corresponding to a biological target; and
the compound is not

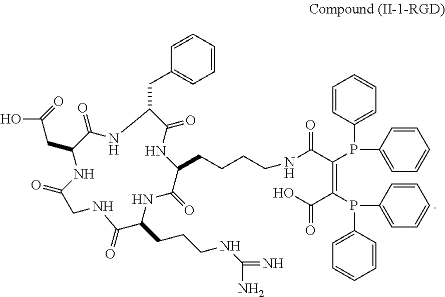

41. A pharmaceutical composition comprising a compound or complex of claim 27 in combination with a pharmaceutically acceptable carrier.

42. A kit for preparing a complex according to claim 38, Compound (Tc-III-1-RGD), Compound (¹⁸⁶Re-III-1-RGD) or Compound (Re-III-1-RGD) comprising a mixture of a reducing agent, a buffering agent, an intermediate co-ligand and a conjugated diphosphine precursor compound or Compound (II-1-RGD);
wherein the conjugated diphosphine precursor compound has a structure according to option i) or ii):
i) the conjugated diphosphine precursor compound has a structure according to Formula (II) 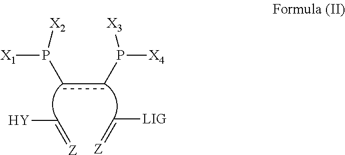
wherein;
each Z is independently O or S;
Y is NH or O;
X₁, X₂, X₃ and X₄ are each independently a substituted or unsubstituted C₅-C₈aryl group, a substituted or unsubstituted 5- to 8-membered heteroaryl group or a substituted or unsubstituted C₃-C₈ cycloalkyl group wherein each substituent is selected from the group consisting of a C₁-C₄alkyl group, C₅-C₁₂aryl or heteroaryl group, a C₁-C₄ acylamido group, a sulfylhydro group, a C₁-C₄ alkylthio group, a C₁-C₄(di)alkylphosphino group, a hydroxy group, a C₁-C₄alkoxy group, a carboxyl group, a C₁-C₄(di)alkylamino group and a C₁-C₄alkoxy-(CH₂CH₂O)ₙ group wherein n is 1, 2, 3, 4, 5, 6, 7, 8, 9 or 10.
the shortest linear chain of carbon atoms between the two Z groups is 4 to 7;
LIG comprises a ligand with a binding motif corresponding to a biological target; and
the compound is not 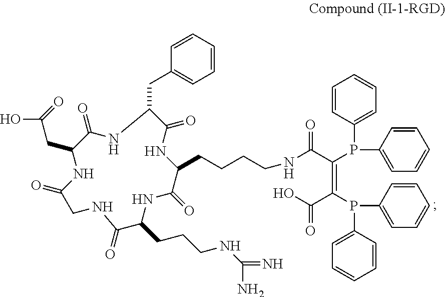
ii) the diphosphine precursor compound has a structure according to Formula (I): 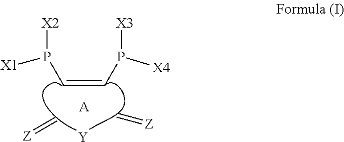
wherein
ring A is a 5, 6, 7 or 8 membered ring;
each Z is independently O or S;
Y is NH or O;
X₁, X₂, X₃ and X₄ are each independently a substituted or unsubstituted C₅-C₈aryl group, a substituted or unsubstituted 5- to 8-membered heteroaryl group wherein any substituents selected from the group consisting of a C₁-C₄alkyl group, C₅-C₁₂aryl or heteroaryl group, a C₁-C₄ acylamido group, a sulfylhydro group, a C₁-C₄ alkylthio group, a C₁-C₄(di)alkylphosphino group, a hydroxy group, a C₁-C₄alkoxy group, a carboxyl group, a C₁-C₄(di)alkylamino group and a C₁-C₄alkoxy-(CH₂CH₂O)ₙ group wherein n is 1, 2, 3, 4, 5, 6, 7, 8, 9 or 10; and
the compound is not

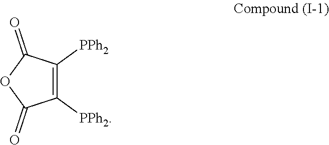

43. The kit of claim 42, wherein the kit further comprising a radionuclide selected from ^{99m}Tc, ²¹²Bi, ²¹³Bi, ¹⁸⁶Re, ¹⁸⁸Re, ⁸⁹Zr, ⁶⁷Ga, ⁶⁸Ga, ⁶⁷Cu, ⁶⁴Cu, ⁶²Cu, ⁶¹Cu, ⁶⁰Cu and ⁵²Mn.

44. The kit according to claim 43, wherein the radionuclide is selected from ^{99m}Tc, ¹⁸⁶Re, ¹⁸⁸Re, and ⁵²Mn.

45. A method of treating or diagnosing a disease comprising administering a compound or complex of claim 27, Compound (I-1), Compound (II-1-RGD), Compound (Tc-III-1-RGD), Compound (¹⁸⁶Re-III-1-RGD) or Compound (Re-III-1-RGD) to a subject.

46. Non-therapeutic use of a compound or complex of claim 27, Compound (I-1), Compound (II-1-RGD), Compound (Tc-III-1-RGD), Compound (¹⁸⁶Re-III-1-RGD) or Compound (Re-III-1-RGD) in imaging or cell labelling.
